SEQUENCE LISTING (250 sequences)

SEQ ID NO: 1 - protein, 8 aa, Artificial Sequence
  TLR4 ligand
GGKSGRTG

SEQ ID NO: 2 - protein, 9 aa, Artificial Sequence
  TLR4 ligand
KGYDWLVVG

SEQ ID NO: 3 - protein, 10 aa, Artificial Sequence
  TLR4 ligand
EDMVYRIGVP

SEQ ID NO: 4 - protein, 6 aa, Artificial Sequence
  TLR4 ligand
VKLSGS

SEQ ID NO: 5 - protein, 8 aa, Artificial Sequence
  TLR4 ligand
GMLSLALF

SEQ ID NO: 6 - protein, 7 aa, Artificial Sequence
  TLR4 ligand
CVVGSVR

SEQ ID NO: 7 - protein, 8 aa, Artificial Sequence
  TLR4 ligand
IVRGCLGW

SEQ ID NO: 8 - protein, 8 aa, Artificial Sequence
  TLR4 ligand
AAEERTLG

SEQ ID NO: 9 - protein, 9 aa, Artificial Sequence
  TLR4 ligand
WARVVGWLR

SEQ ID NO: 10 - protein, 9 aa, Artificial Sequence
  TLR4 ligand
SEGYRLFGG

SEQ ID NO: 11 - protein, 10 aa, Artificial Sequence
  TLR4 ligand
LVGGVVRRGS

SEQ ID NO: 12 - protein, 10 aa, Artificial Sequence
  TLR4 ligand
GRVNDLWLAA

SEQ ID NO: 13 - protein, 10 aa, Artificial Sequence
  TLR4 ligand
SGWMLWREGS

SEQ ID NO: 14 - protein, 10 aa, Artificial Sequence
  TLR4 ligand
ERMEDRGGDL

SEQ ID NO: 15 - protein, 9 aa, Artificial Sequence
  TLR4 ligand
KLCCFTECM

SEQ ID NO: 16 - protein, 10 aa, Artificial Sequence
  TLR4 ligand
AVGSMERGRG

SEQ ID NO: 17 - protein, 9 aa, Artificial Sequence
  TLR4 ligand
RDWVGGDLV

SEQ ID NO: 18 - protein, 10 aa, Artificial Sequence
  TLR4 ligand
FFEVAKISQQ

SEQ ID NO: 19 - protein, 5 aa, Artificial Sequence
  TLR4 ligand
WWYWC

SEQ ID NO: 20 - protein, 7 aa, Artificial Sequence
  TLR4 ligand
MHLCSHA

SEQ ID NO: 21 - protein, 7 aa, Artificial Sequence
  TLR4 ligand
WLFRRIG

SEQ ID NO: 22 - protein, 7 aa, Artificial Sequence
  TLR4 ligand
YWFWRIG

SEQ ID NO: 23 - protein, 7 aa, Artificial Sequence
  TLR4 ligand
MHLYCIA

SEQ ID NO: 24 - protein, 8 aa, Artificial Sequence
  TLR4 ligand
WPLFPWIV

SEQ ID NO: 25 - protein, 7 aa, Artificial Sequence
  TLR4 ligand
DMRSHAR

SEQ ID NO: 26 - protein, 7 aa, Artificial Sequence
  TLR4 ligand
MHLCTHA

SEQ ID NO: 27 - protein, 6 aa, Artificial Sequence
  TLR4 ligand
NLFPFY

SEQ ID NO: 28 - protein, 7 aa, Artificial Sequence
  TLR4 ligand
MHLCTRA

SEQ ID NO: 29 - protein, 7 aa, Artificial Sequence
  TLR4 ligand
RHLWYHA

SEQ ID NO: 30 - protein, 7 aa, Artificial Sequence
  TLR4 ligand
WPFSAYW

SEQ ID NO: 31 - protein, 6 aa, Artificial Sequence
  TLR4 ligand
WYLRGS

SEQ ID NO: 32 - protein, 7 aa, Artificial Sequence
  TLR4 ligand
GKGTDLG

SEQ ID NO: 33 - protein, 6 aa, Artificial Sequence
  TLR4 ligand
IFVRMR

SEQ ID NO: 34 - protein, 7 aa, Artificial Sequence
  TLR4 ligand
WLFRPVF

SEQ ID NO: 35 - protein, 7 aa, Artificial Sequence
  TLR4 ligand
FLGWLMG

SEQ ID NO: 36 - protein, 7 aa, Artificial Sequence
  TLR4 ligand
MHLWHHA

SEQ ID NO: 37 - protein, 7 aa, Artificial Sequence
  TLR4 ligand
WWFPWKA

SEQ ID NO: 38 - protein, 7 aa, Artificial Sequence
  TLR4 ligand
WYLPWLG

SEQ ID NO: 39 - protein, 7 aa, Artificial Sequence
  TLR4 ligand
WPFPRTF

SEQ ID NO: 40 - protein, 7 aa, Artificial Sequence
  TLR4 ligand
WPFPAYW

SEQ ID NO: 41 - protein, 7 aa, Artificial Sequence
  TLR4 ligand
FLGLRWL

SEQ ID NO: 42 - protein, 10 aa, Artificial Sequence
  TLR4 ligand
SRTDVGVLEV

SEQ ID NO: 43 - protein, 10 aa, Artificial Sequence
  TLR4 ligand
REKVSRGDKG

SEQ ID NO: 44 - protein, 10 aa, Artificial Sequence
  TLR4 ligand
DWDAVESEYM

SEQ ID NO: 45 - protein, 10 aa, Artificial Sequence
  TLR4 ligand
VSSAQEVRVP

SEQ ID NO: 46 - protein, 10 aa, Artificial Sequence
  TLR4 ligand
LTYGGLEALG

SEQ ID NO: 47 - protein, 10 aa, Artificial Sequence
  TLR4 ligand
VEEYSSSGVS

SEQ ID NO: 48 - protein, 10 aa, Artificial Sequence
  TLR4 ligand
VCEVSDSVMA

SEQ ID NO: 49 - protein, 5 aa, Artificial Sequence
  TLR2 ligand
NPPTT

SEQ ID NO: 50 - protein, 5 aa, Artificial Sequence
  TLR2 ligand
MRRIL

SEQ ID NO: 51 - protein, 4 aa, Artificial Sequence
  TLR2 ligand
MISS

SEQ ID NO: 52 - protein, 5 aa, Artificial Sequence
  TLR2 ligand
RGGSK

SEQ ID NO: 53 - protein, 4 aa, Artificial Sequence
  TLR2 ligand
RGGF

SEQ ID NO: 54 - protein, 5 aa, Artificial Sequence
  TLR2 ligand
NRTVF

SEQ ID NO: 55 - protein, 5 aa, Artificial Sequence
  TLR2 ligand
NRFGL

SEQ ID NO: 56 - protein, 5 aa, Artificial Sequence
  TLR2 ligand
SRHGR

SEQ ID NO: 57 - protein, 5 aa, Artificial Sequence
  TLR2 ligand
IMRHP

SEQ ID NO: 58 - protein, 5 aa, Artificial Sequence
  TLR2 ligand
EVCAP

SEQ ID NO: 59 - protein, 5 aa, Artificial Sequence
  TLR2 ligand
ACGVY

SEQ ID NO: 60 - protein, 5 aa, Artificial Sequence
  TLR2 ligand
CGPKL

SEQ ID NO: 61 - protein, 5 aa, Artificial Sequence
  TLR2 ligand
AGCFS

SEQ ID NO: 62 - protein, 5 aa, Artificial Sequence
  TLR2 ligand
SGGLF

SEQ ID NO: 63 - protein, 5 aa, Artificial Sequence
  TLR2 ligand
AVRLS

SEQ ID NO: 64 - protein, 5 aa, Artificial Sequence
  TLR2 ligand
GGKLS

SEQ ID NO: 65 - protein, 5 aa, Artificial Sequence
  TLR2 ligand
VSEGV

SEQ ID NO: 66 - protein, 5 aa, Artificial Sequence
  TLR2 ligand
KCQSF

SEQ ID NO: 67 - protein, 5 aa, Artificial Sequence
  TLR2 ligand
FCGLG

SEQ ID NO: 68 - protein, 5 aa, Artificial Sequence
  TLR2 ligand
PESGV

SEQ ID NO: 69 - protein, 5 aa, Artificial Sequence
  TLR2 ligand
DPDSG

SEQ ID NO: 70 - protein, 5 aa, Artificial Sequence
  TLR2 ligand
IGRFR

SEQ ID NO: 71 - protein, 5 aa, Artificial Sequence
  TLR2 ligand
MGTLP

SEQ ID NO: 72 - protein, 5 aa, Artificial Sequence
  TLR2 ligand
ADTHQ

SEQ ID NO: 73 - protein, 5 aa, Artificial Sequence
  TLR2 ligand
HLLPG

SEQ ID NO: 74 - protein, 5 aa, Artificial Sequence
  TLR2 ligand
GPLLH

SEQ ID NO: 75 - protein, 5 aa, Artificial Sequence
  TLR2 ligand
NYRRW

SEQ ID NO: 76 - protein, 5 aa, Artificial Sequence
  TLR2 ligand
LRQGR

SEQ ID NO: 77 - protein, 5 aa, Artificial Sequence
  TLR2 ligand
IMWFP

SEQ ID NO: 78 - protein, 5 aa, Artificial Sequence
  TLR2 ligand
RVVAP

SEQ ID NO: 79 - protein, 5 aa, Artificial Sequence
  TLR2 ligand
IHVVP

SEQ ID NO: 80 - protein, 5 aa, Artificial Sequence
  TLR2 ligand
MFGVP

SEQ ID NO: 81 - protein, 5 aa, Artificial Sequence
  TLR2 ligand
CVWLQ

SEQ ID NO: 82 - protein, 5 aa, Artificial Sequence
  TLR2 ligand
IYKLA

SEQ ID NO: 83 - protein, 4 aa, Artificial Sequence
  TLR2 ligand
KGWF

SEQ ID NO: 84 - protein, 5 aa, Artificial Sequence
  TLR2 ligand
KYMPH

SEQ ID NO: 85 - protein, 5 aa, Artificial Sequence
  TLR2 ligand
VGKND

SEQ ID NO: 86 - protein, 5 aa, Artificial Sequence
  TLR2 ligand
THKPK

SEQ ID NO: 87 - protein, 5 aa, Artificial Sequence
  TLR2 ligand
SHIAL

SEQ ID NO: 88 - protein, 5 aa, Artificial Sequence
  TLR2 ligand
AWAGT

SEQ ID NO: 89 - protein, 20 aa, Artificial Sequence
  TLR2 ligand
KGGVGPVRRSSRLRRTTQPG

SEQ ID NO: 90 - protein, 23 aa, Artificial Sequence
  TLR2 ligand
GRRGLCRGCRTRGRIKQLQSAHK

SEQ ID NO: 91 - protein, 22 aa, Artificial Sequence
  TLR2 ligand
RWGYHLRDRKYKGVRSHKGVPR

SEQ ID NO: 92 - protein, 23 aa, Artificial Sequence
  OprI lipoprotein
MNNVLKFSALALAAVLATGCSSH

SEQ ID NO: 93 - DNA, 72 bp, Artificial Sequence
  OprI lipoprotein
atgaaagctactaaactggtactgggcgcggtaatcctgggttctactctgctggcaggttgctccagcaac

SEQ ID NO: 94 - protein, 24 aa, Artificial Sequence
  OprI lipoprotein
MKATKLVLGAVILGSTLLAGCSSN

SEQ ID NO: 95 - DNA, 72 bp, Artificial Sequence; identical to SEQ ID NO: 93
  OprI lipoprotein

SEQ ID NO: 96 - protein, 5 aa, Artificial Sequence
  TLR agonist
GGKLS

SEQ ID NO: 97 - protein, 5 aa, Artificial Sequence
  TLR agonist
FCGLG

SEQ ID NO: 98 - protein, 506 aa, Artificial Sequence
  Flagellin
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 99 - protein, 494 aa, Artificial Sequence
  Flagellin
AQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNVQQKYKVSDTAATVTGYADTTIALDNSTFKASATGLGGTDQKIDGDLKFDDTTGKYYAKVTVTGGTGKDGYYEVSVDKTNGEVTLAGGATSPLTGGLPATATEDVKNVQVANADLTEAKAALTAAGVTGTASVVKMSYTDNNGKTIDGGLAVKVGDDYYSATQNKDGSISINTTKYTADDGTSKTALNKLGGADGKTEVVSIGGKTYAASKAEGHNFKAQPDLAEAAATTTENPLQKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 100 - protein, 506 aa, Artificial Sequence; identical to SEQ ID NO: 98
  Flagellin

SEQ ID NO: 101 - protein, 277 aa, Artificial Sequence
  Flagellin
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVHGAPVDPASPWTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 102 - protein, 506 aa, Artificial Sequence
  Flagellin
MAQVINTNSLSLLTQNNLNKSQSALGTAIERLSSGLRINSAKDDAAGQAIANRFTANIKGLTQASRNANDGISIAQTTEGALNEINNNLQRVRELAVQSANGTNSQSDLDSIQAEITQRLNEIDRVSGQTQFNGVKVLAQDNTLTIQVGANDGETIDIDLKEISSKTLGLDKLNVQDAYTPKETAVTVDKTTYKNGTDTITAQSNTDIQTAIGGGATGVTGADIKFKDGQYYLDVKGGASAGVYKATYDETTKKVNIDTTDKTPLATAEATAIRGTATITHNQIAEVTKEGVDTTTVAAQLAAAGVTGADKDNTSLVKLSFEDKNGKVIDGGYAVKMGDDFYAATYDEKTGTITAKTTTYTDGAGVAQTGAVKFGGANGKSEVVTATDGKTYLASDLDKHNFRTGGELKEVNTDKTENPLQKIDAALAQVDTLRSDLGAVQNRFNSAITNLGNTVNNLSSARSRIEDSDYATEVSNMSRAQILQQAGTSVLAQANQVPQNVLSLLR

SEQ ID NO: 103 - protein, 504 aa, Artificial Sequence
  Flagellin
AQVINTNSLSLLTQNNLNKSQSALGTAIERLSSGLRINSAKDDAAGQAIANRFTANIKGLTQASRNANDGISIAQTTEGALNEINNNLQRVRELAVQSANGTNSQSDLDSIQAEITQRLNEIDRVSGQTQFNGVKVLAQDNTLTIQVGANDGETIDIDLKEISSKTLGLDKLNVQDAYTPKETAVTVDKTTYKNGTDTITAQSNTDIQTAIGGGATGVTGADIKFKDGQYYLDVKGGASAGVYKATYDETTKKVNIDTTDKTPLATAEATAIRGTATITHNQIAEVTKEGVDTTTVAAQLAAAGVTGADKDNTSLVKLSFEDKNGKVIDGGYAVKMGDDFYAATYDEKQVQLLLNNHYTDGAGVLQTGAVKFGGANGKSEVVTATVGKTYLASDLDKHNFRTGGELKEVNTDKTENPLQKIDAALAQVDTLRSDLGAVQNRFNSAITNLGNTVNNLSSARSRIEDSDYATEVSNMSRAQILQQAGTSVLAQANQVPQNVLSLLR

SEQ ID NO: 104 - protein, 393 aa, Artificial Sequence
  Flagellin
ALTVNTNIASLNTQRNLNNSSASLNTSLQRLSTGSRINSAKDDAAGLQIANRLTSQVNGLNVATKNANDGISLAQTAEGALQQSTNILQRMRDLSLQSANGSNSDSERTALNGEVKQLQKELDRISNTTTFGGRKLLDGSFGVASFQVGSAANEIISVGIDEMSAESLNGTYFKADGGGAVTAATASGTVDIAIGITGGSAVNVKVDMKGNETAEQAAAKIAAAVNDANVGIGAFSDGDTISYVSKAGKDGSGAITSAVSGVVIADTGSTGVGTAAGVTPSATAFAKTNDTVAKIDISTAKGAQSAVLVIDEAIKQIDAQRADLGAVQNRFDNTINNLKNIGENVSAARGRIEDTDFAAETANLTKNQVLQQAGTAILAQANQLPQSVLSLLR

SEQ ID NO: 105 - protein, 287 aa, Artificial Sequence
  Flagellin
MKVNTNIISLKTQEYLRKNNEGMTQAQERLASGKRINSSLDDAAGLAVVTRMNVKSTGLDAASKNSSMGIDLLQTADSALSSMSSILQRMRQLAVQSSNGSFSDEDRKQYTAEFGSLIKELDHVADTTNYNNIKLLDQTATNAATQVSIQASDKANDLINIDLFNAKGLSAGTITLGSGSTVAGYSALSVADADSSQEATEAIDELINNISNGRALLGAGMSRLSYNVSNVNNQSIATKASASSIEDADMAAEMSEMTKYKILTQTSISMLSQANQTPQMLTQLINS

SEQ ID NO: 106 - protein, 595 aa, Artificial Sequence
  Flagellin
MAQVINTNSLSLITQNNINKNQSALSSSIERLSSGLRINSAKDDAAGQAIANRFTSNIKGLTQAARNANDGISVAQTTEGALSEINNNLQRIRELTVQASTGTNSDSDLDSIQDEIKSRLDEIDRVSGQTQFNGVNVLAKDGSMKIQVGANDGQTITIDLKKIDSDTLGLNGFNVNGSGTIANKAATISDLTAAKMDAATNTITTTNNALTASKALDQLKDGDTVTIKADAAQTATVYTYNASAGNFSLSNVSNNTSEKAGDVAASLLPPAGQTASGVYKAASGEVNFDVDANGKITIGGQKAYLTSDGNLTTNDAGGATAATLDGLFKKAGDGQSIGFKKTASVTMGGTTYNFKTGADADAATANAGVSFTDTASKETVLNKVATAKQGKAAAADGDTSATITYKSGVQTYQAVFAAGDGTASAKYADKADVSNATATYTDADGEMTTIGSYTTKYSIDANNGKVTVDSGTGTGKYAPKVGAEVYVSANGTLTTDATSEGTVTKDPLKALDEAISSIDKFRSSLGAIQNRLDSAVTNLNNTTTNLSEAQSRIQDADYATEVSNMSKAQIIQQAGNSVLAKANQVPQQVLSLLQG

SEQ ID NO: 107 - protein, 497 aa, Artificial Sequence
  Flagellin
AQVINTNSLSLITQNNINKNQSALSSSIERLSSGLRINSAKDDAAGQAIANRFTSNIKGLTQAARNANDGISVAQTTEGALSEINNNLQRVRELTVQATTGTNSESDLSSIQDEIKSRLDEIDRVSGQTQFNGVNVLAKNGSMKIQVGANDNQTITIDLKQIDAKTLGLDGFSVKNNDTVTTSAPVTAFGATTTNNIKLTGITLSTEAATDTGGTNPASIEGVYTDNGNDYYAKITGGDNDGKYYAVTVANDGTVTMATGATANATVTDANTTKATTITSGGTPVQIDNTAGSATANLGAVSLVKLQDSKGNDTDTYALKDTNGNLYAADVNETTGAVSVKTITYTDSSGAASSPTAVKLGGDDGKTEVVDIDGKTYDSADLNGGNLQTGLTAGGEALTAVANGKTTDPLKALDDAIASVDKFRSSLGAVQNRLDSAVTNLNNTTTNLSEAQSRIQDADYATEVSNMSKAQIIQQAGNSVLAKANQVPQQVLSLLQG

SEQ ID NO: 108 - DNA, 1518 bp, Artificial Sequence
  Flagellin
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaaaaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaactacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattctgcagcaggccggtacttccgttctggcgcaggctaaccaggtcccgcagaacgtgctgtctctgttacgt

SEQ ID NO: 109 - DNA, 832 bp, Artificial Sequence
  Flagellin
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcatggagcgccggtggatcctgctagcccatggaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgtctctgttacgtg

SEQ ID NO: 110 - DNA, 1522 bp, Artificial Sequence
  Flagellin
aatggcacaagtcattaatacaaacagcctgtcgctgttgacccagaataacctgaacaaatcccagtccgctctgggcaccgctatcgagcgtctgtcttccggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacggtactaactcccagtctgaccttgactctatccaggctgaaatcacccagcgtctgaacgaaatcgaccgtgtatccggtcagactcagttcaacggcgtgaaagtcctggcgcaggacaacaccctgaccatccaggttggtgccaacgacggtgaaactattgatattgatttaaaagaaattagctctaaaacactgggacttgataagcttaatgtccaggatgcctacaccccgaaagaaactgctgtaaccgttgataaaactacctataaaaatggtacagatactattacagcccagagcaatactgatatccaaactgcaattggcggtggtgcaacgggggttactggggctgatatcaaatttaaagatggtcaatactatttagatgttaaaggcggtgcttctgctggtgtttataaagccacttatgatgaaactacaaagaaagttaatattgatacgactgataaaactccgttagcaactgcggaagctacagctattcggggaacggccactataacccacaaccaaattgctgaagtaacaaaagagggtgttgatacgaccacagttgcggctcaacttgctgctgcaggggttactggtgccgataaggacaatactagccttgtaaaactatcgtttgaggataaaaacggtaaggttattgatggtggctatgcagtgaaaatgggcgacgatttctatgccgctacatatgatgagaaaacaggtacaattactgctaaaacaaccacttatacagatggtgctggcgttgctcaaactggagctgtgaaatttggtggcgcaaatggtaaatctgaagttgttactgctaccgatggtaaaacttacttagcaagcgaccttgacaaacataacttcagaacaggcggtgagcttaaagaggttaatacagataagactgaaaacccactgcagaaaattgatgctgccttggcacaggttgatacacttcgttctgacctgggtgcggtacagaaccgtttcaactccgctatcaccaacctgggcaataccgtaaataacctgtcttctgcccgtagccgtatcgaagattccgactacgcgaccgaagtctccaacatgtctcgcgcgcagattctgcagcaggccggtacctccgttctggcgcaggctaaccaggttccgcaaaacgtcctctctttactgcgttaa

SEQ ID NO: 111 - DNA, 1788 bp, Artificial Sequence
  Flagellin
atggcacaagtcattaataccaacagcctctcgctgatcactcaaaataatatcaacaagaaccagtctgcgctgtcgagttctatcgagcgtctgtcttctggcttgcgtattaacagcgcgaaggatgacgccgcaggtcaggcgattgctaaccgttttacttctaacattaaaggcctgactcaggctgcacgtaacgccaacgacggtatttccgttgcgcagaccaccgaaggcgcgctgtccgaaatcaacaacaacttacagcgtatccgtgaactgacggttcaggcttctaccgggactaactccgattcagatctggactccattcaggacgaaatcaaatcccgtctggacgaaattgaccgcgtatctggccagacccagttcaacggcgtgaacgtactggcgaaagacggttcaatgaaaattcaggttggtgcgaatgacggccagactatcacgattgatctgaagaaaattgactcagatacgctggggctgaatggttttaacgtgaatggttccggtacgatagccaataaagcggcgaccattagcgacctgacagcagcgaaaatggatgctgcaactaatactataactacaacaaataatgcgctgactgcatcaaaggcgcttgatcaactgaaagatggtgacactgttactatcaaagcagatgctgctcaaactgccacggtttatacatacaatgcatcagctggtaacttctcactcagtaatgtatcgaataatacttcagaaaaagcaggtgatgtagcagctagccttctcccgccggctgggcaaactgctagtggtgtttataaagcagcaagcggtgaagtgaactttgatgttgatgcgaatggtaaaatcacaatcggaggacagaaagcatatttaactagtgatggtaacttaactacaaacgatgctggtggtgcgactgcggctacgcttgatggtttattcaagaaagctggtgatggtcaatcaatcgggtttaagaagactgcatcagtcacgatggggggaacaacttataactttaaaacgggtgctgatgctgatgctgcaactgctaacgcaggggtatcgttcactgatacagctagcaaagaaaccgttttaaataaagtggctacagctaaacaaggcaaagcagctgcagctgacggtgatacatccgcaacaattacctataaatctggcgttcagacgtatcaggctgtatttgccgcaggtgacggtactgctagcgcaaaatatgccgataaagctgacgtttctaatgcaacagcaacatacactgatgctgatggtgaaatgactacaattggttcatacaccacgaagtattcaatcgatgctaacaacggcaaggtaactgttgattctggaactggtacgggtaaatatgcgccgaaagtaggggctgaagtatatgttagtgctaatggtactttaacaacagatgcaactagcgaaggcacagtaacaaaagatccactgaaagctctggatgaagctatcagctccatcgacaaattccgttcttccctgggtgctatccagaaccgtctggattccgcagtcaccaacctgaacaacaccactaccaacctgtccgaagcgcagtcccgtattcaggacgccgactatgcgaccgaagtgtccaacatgtcgaaagcgcagatcattcagcaggccggtaactccgtgctggcaaaagccaaccaggtaccgcagcaggttctgtctctgctgcagggttag

SEQ ID NO: 112 - protein, 4 aa, Artificial Sequence
  Flagellin
TIAL

SEQ ID NO: 113 - protein, 505 aa, Artificial Sequence
  Flagellin
AQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 114 - protein, 4 aa, Artificial Sequence
  Flagellin
TTLD

SEQ ID NO: 115 - protein, 7 aa, Artificial Sequence
  Flagellin
GTDQKID

SEQ ID NO: 116 - protein, 6 aa, Artificial Sequence
  Flagellin
NGEVTL

SEQ ID NO: 117 - protein, 5 aa, Artificial Sequence
  Flagellin
GADAA

SEQ ID NO: 118 - protein, 4 aa, Artificial Sequence
  Flagellin
AGGA

SEQ ID NO: 119 - protein, 4 aa, Artificial Sequence
  Flagellin
PATA

SEQ ID NO: 120 - protein, 4 aa, Artificial Sequence
  Flagellin
ATTK

SEQ ID NO: 121 - protein, 12 aa, Artificial Sequence
  Flagellin
AGATKTTMPAGA

SEQ ID NO: 122 - protein, 5 aa, Artificial Sequence
  Flagellin
VTGTG

SEQ ID NO: 123 - protein, 10 aa, Artificial Sequence
  Flagellin
TEAKAALTAA

SEQ ID NO: 124 - protein, 11 aa, Artificial Sequence
  Flagellin
ASVVKMSYTDN

SEQ ID NO: 125 - protein, 9 aa, Artificial Sequence
  Flagellin
VDATDANGA

SEQ ID NO: 126 - protein, 4 aa, Artificial Sequence
  Flagellin
AAGA

SEQ ID NO: 127 - protein, 535 aa, Artificial Sequence
  CRM197
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 128 - protein, 164 aa, Artificial Sequence
  TMV coat protein
MMAYSIPTPSQLVYFTENYADYIPFVNRLINARSNSFQTQSGRDELREILIKSQVSVVSPISRFPAEPAYYIYLRDPSISTVYTALLQSTDTRNRVIEVENSTNVTTAEQLNAVRRTDDASTAIHNNLEQLLSLLTNGTGVFNRTSFESASGLTWLVTTTPRTA

SEQ ID NO: 129 - protein, 221 aa, Artificial Sequence
  AMV
MSSSQKKAGGKAGKPTKRSQNYAALRKAQLPKPPALKVPVAKPTNTILPQTGCVWQSLGTPLSLSSSNGLGARFLYSFLKDFAAPRILEEDLIFRMVFSITPSHAGSFCLTDDVTTEDGRAVAHGNPMQEFPHGAFHANEKFGFELVFTAPTHAGMQNQNFKHSYAVALCLDFDALPEGSRNPSYRFNEVWVERKAFPRAGPLRSLITVGLFDDADDLDRQ

SEQ ID NO: 130 - protein, 237 aa, Artificial Sequence
  coat protein of Potato Virus x
MTTPANTTQATGSTTSTTTKTAGATPATTSGLFTIPDGEFFSTARAIVASNAVATNEDLSKIEAIWKDMKVPTDTMAQAAWDLVRHCADVGSSAQTEMIDTGPYSNGISRARLAAAIKEVCTLRQFCMKYAPVVWNWMLTNNSPPANWQAQGFKPEHKFAAFDFFNGVTNPAAIMPKEGLIRPPSEAEMNAAQTAAFVKITKARAQSNDFASLDAAVTRGRITGTTTAEAVVTLPPP

SEQ ID NO: 131 - protein, 395 aa, Artificial Sequence
  Class I OMP
MRKKLTALVLSALPLAAVADVSLYGEIKAGVEGRNYQLQLTEAQAANGGASGQVKVTKVTKAKSRIRTKISDFGSFIGFKGSEDLGEGLKAVWQLEQDVSVAGGGATQWGNRESFIGLAGEFGTLRAGRVANQFDDASQAIDPWDSNNDVASQLGIFKRHDDMPVSVRYDSPEFSGFSGSVQFVPAQNSKSAYKPAYWTTVNTGSATTTTFVPAVVGKPGSDVYYAGLNYKNGGFAGNYAFKYARHANVGRDAFELFLLGSGSDQAKGTDPLKNHQVHRLTGGYEEGGLNLALAAQLDLSENGDKTKNSTTEIAATASYRFGNAVPRISYAHGFDFIERGKKGENTSYDQIIAGVDYDFSKRTSAIVSGAWLKRNTGIGNYTQINAASVGLRHKF

SEQ ID NO: 132 - protein, 347 aa, Artificial Sequence
  Fimbrillin
MVLKTSNSNRAFGVGDDESKVAKLTVMVYNGEQQEAIKSAENATKVEDIKCSAGQRTLVVMANTGAMELVGKTLAEVKALTTELTAENQEAAGLIMTAEPKTIVLKAGKNYIGYSGTGEGNHIENDPLKIKRVHARMAFTEIKVQMSAAYDNIYTFVPEKIYGLIAKKQSNLFGATLVNADANYLTGSLTTFNGAYTPANYANVPWLSRNYVAPAADAPQGFYVLENDYSANGGTIHPTILCVYGKLQKNGADLAGADLAAAQAANWVDAEGKTYYPVLVNFNSNNYTYDSNYTPKNKIERNHKYDIKLTITGPGTNNPENPITESAHLNVQCTVAEWVLVGQNATW

SEQ ID NO: 133 - protein, 428 aa, Artificial Sequence
  MALP-2
MKKSKKILLGLSPIAAVLPAVAVSCGNNDESNISFKEKDISKYTTTNANGKQVVKNAELLKLKPVLITDEGKIDDKSFNQSAFEALKAINKQTGIEINSVEPSSNFESAYNSALSAGHKIWVLNGFKHQQSIKQYIDAHREELERNQIKIIGIDFDIETEYKWFYSLQFNIKESAFTTGYAIASWLSEQDESKRVVASFGVGAFPGVTTFNEGFAKGILYYNQKHKSSKIYHTSPVKLDSGFTAGEKMNTVINNVLSSTPADVKYNPHVILSVAGPATFETVRLANKGQYVIGVDSDQGMIQDKDRILTSVLKHIKQAVYETLLDLILEKEEGYKPYVVKDKKADKKWSHFGTQKEKWIGVAENHFSNTEEQAKINNKIKEAIKMFKELPEDFVKYINSDKALKDGNKIDNVSERLEAIISAINKAAK

SEQ ID NO: 134 - protein, 143 aa, Artificial Sequence
  p19 protein
ATTLPVQRHPRSLFPEFSELFAAFPSFAGLRPTFDTRLMRLEDEMKEGRYEVRAELPGVDPDKDVDIMVRDGQLTIKAERTEQKDFDGRSEFAYGSFVRTVSLPVGADEDDIKATYDKGILTVSVAVSEGKPTEKHIQIRSTN

SEQ ID NO: 135 - protein, 277 aa, Artificial Sequence; identical to SEQ ID NO: 101
  Flagellin without a hinge region (STF2 delta)

SEQ ID NO: 136 - protein, 158 aa, Artificial Sequence
  HPV16 E6
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 137 - protein, 140 aa, Artificial Sequence
  HPV E6 protein
MATPIRTVRQLSESLCIPYIDVLLPCNFCNYFLSNAEKLLFDHFDLHLVWRDNLVFGCCQGCARTVSLLEFVLYYQESYEVPEIEEILDRPLLQIELRCVTCIKKLSVAEKLEVVSNGERVHRVRNRLKAKCSLCRLYAI

SEQ ID NO: 138 - DNA, 420 bp, Artificial Sequence
  E6 protein
atggcgacaccaatccggaccgtcagacagctttccgaaagcctctgtatcccatatattgatgttttattgccttgtaatttttgtaattattttttgtctaatgctgagaagctgctttttgatcattttgatttgcatcttgtctggagagacaatttggtgtttggatgctgtcaagggtgtgctagaactgttagcctattggagtttgttttatattatcaggagtcttatgaggtaccggaaatagaagaaattttggacagacctttattgcaaattgaactccgttgtgttacatgcataaaaaaactgagtgttgctgaaaaattggaggttgtgtcaaacggagaaagagtgcatagagttagaaacagacttaaagcaaagtgtagtttgtgtcgcttgtatgctata

SEQ ID NO: 139 - protein, 159 aa, Artificial Sequence
  HPV E6 protein
MHTRAGMSEENPCPRNIFLLCKEYGLELEDLRLLCVWCKRPLSEADIWAFAIKELFVVWRKGFPFGACGKCLIAAGKLRQYRHWHYSCYGDTVETETGIPIPQLFMRCYICHKPLSWEEKEALLVGNKRFHNISGRWTGHCMNCGSSCTATDPASRTLH

SEQ ID NO: 140 - DNA, 476 bp, Artificial Sequence
  E6 protein
atgcacacaagggcagggatgtctgaggagaatccatgccctaggaacatctttttgctttgcaaagagtatggtttggagctagaggatttgcgattgctctgtgtatggtgcaaacggccgttatcagaggctgacatatgggcatttgcaataaaagaactgtttgtagtgtggagaaagggcttcccatttggagcctgcggaaaatgcctgattgcagcaggaaaacttagacaatacagacattggcattactcatgctacggagacacagtggagactgagacaggaatacccatacctcagctgtttatgagatgctatatttgccataagcccctgagctgggaggagaaggaggcattactagttggaaacaagcgtttccacaacatatcaggccggtggacgggacattgcatgaactgtgggtcatcatgcacggcaaccgacccagcctcaaggacattaca

SEQ ID NO: 141 - protein, 157 aa, Artificial Sequence
  HPV5 E6 protein
MAEGAEHQQKLTEKDKAELPSTIRDLAETLGIPLIDCIIPCNFCGKFLNYLEACEFDYKKLSLIWKDYCVFACCRVCCGATATYEFNQFYEQTVLGRDIELASGLSIFDIDIRCQTCLAFLDIIEKLDCCGRGLPFHKVRNAWKGICRQCKHFYHDW

SEQ ID NO: 142 - DNA, 471 bp, Artificial Sequence
  HPV5 E6 protein
atggctgagggagccgaacaccaacagaaactgacagaaaaagataaggcagaattaccttcaaccattagagacttagctgaaaccttaggcatccctcttattgattgtataataccttgcaatttttgtggtaaatttttaaattatttggaagcctgtgaattcgactacaaaaaacttagcctaatttggaaagattattgtgtgtttgcgtgttgtcgcgtatgctgtggcgccactgcaacatacgaatttaatcaattttatgagcagacagtgttaggaagagatattgagttagcttcaggactctcgatttttgatattgatatcaggtgtcaaacttgcttagcatttcttgacattatagaaaagttagattgctgtggcagaggccttccctttcacaaggtgaggaacgcctggaagggaatctgtaggcagtgtaagcatttttatcatgattgg

SEQ ID NO: 143 - protein, 150 aa, Artificial Sequence
  HPV6 E6 protein
MESANASTSATTIDQLCKTFNLSMHTLQINCVFCKNALTTAEIYSYAYKQLKVLFRGGYPYAACACCLEFHGKINQYRHFDYAGYATTVEEETKQDILDVLIRCYLCHKPLCEVEKVKHILTKARFIKLNCTWKGRCLHCWTTCMEDMLP

SEQ ID NO: 144 - DNA, 450 bp, Artificial Sequence
  HPV6 E6 protein
atggaaagtgcaaatgcctccacgtctgcaacgaccatagaccagttgtgcaagacgtttaatctatctatgcatacgttgcaaattaattgtgtgttttgcaagaatgcactgaccactgcagagatttattcatatgcatataaacagctaaaggtcctgtttcgaggcggctatccatatgcagcctgcgcgtgctgcctagaatttcatggaaaaattaaccaatatagacactttgattatgctggatatgcaacaactgttgaagaagaaactaaacaagacattttagacgtgctaattcggtgctacctgtgtcacaaaccgctgtgtgaagtagaaaaggtaaaacatatactaaccaaggcacggttcataaagctaaattgtacgtggaagggtcgctgcctacactgctggacaacatgcatggaagacatgttaccc

SEQ ID NO: 145 - protein, 120 aa, Artificial Sequence
  HPV8 E6 protein
STIKELAEALGIPLQDCLVPCNFCGNFFDFLELCEFDKKRLCLIWKNYVVTACCRCCCVATATFEFNEYYEQTVLGRDIELATGRSIFEIDVRCQNCLSFLDIIEKLDCCGRGRPFHKVR

SEQ ID NO: 146 - DNA, 361 bp, Artificial Sequence
  HPV8 E6 protein
tctactattaaagagttagctgaggcattaggtattccactgcaggactgtttagtaccttgcaacttttgtggtaactttttcgatttcttagaactgtgtgagtttgacaaaaagagactgtgcctaatttggaaaaattacgttgttactgcgtgttgtcgttgttgttgtgtagcaaccgcaacgtttgaatttaatgaatattatgagcaaactgtgctaggcagagatattgaattagctacaggacgttcaatttttgagatagacgttaggtgtcaaaactgcttgtcatttttggatatcatagagaaattagattgctgtgggagaggccgtccctttcataaagttagag

SEQ ID NO: 147 - protein, 150 aa, Artificial Sequence
  HPV11 E6 protein
MESKDASTSATSIDQLCKTFNLSLHTLQIQCVFCRNALTTAEIYAYAYKNLKVVWRDNFPFAACACCLELQGKINQYRHFNYAAYAPTVEEETNEDILKVLIRCYLCHKPLCEIEKLKHILGKARFIKLNNQWKGRCLHCWTTCMEDLLP

SEQ ID NO: 148 - DNA, 450 bp, Artificial Sequence
  HPV11 E6 protein
atggaaagtaaagatgcctccacgtctgcaacatctatagaccagttgtgcaagacgtttaatctttctttgcacactctgcaaattcagtgcgtgttttgcaggaatgcactgaccaccgcagagatatatgcatatgcctataagaacctaaaggttgtgtggcgagacaactttccctttgcagcgtgtgcctgttgcttagaactgcaagggaaaattaaccaatatagacactttaattatgctgcatatgcacctacagtagaagaagaaaccaatgaagatattttaaaagtgttaattcgttgttacctgtgtcacaagccgttgtgtgaaatagaaaaactaaagcacatattgggaaaggcacgcttcataaaactaaataaccagtggaagggtcgttgcttacactgctggacaacatgcatggaagacttgttaccc

SEQ ID NO: 149 - protein, 158 aa, Artificial Sequence
  HPV18 E6 protein
MARFEDPTRRPYKLPDLCTELSTSLQDIEITCVYCKTVLELTEVFEFAFKDLFVVYRDSIPHAACHKCIDFYSRIRELRHYSDSVYGDTLEKLTNTGLYNLLIRCLRCQKPLNPAEKLRHLNEKRRFHNIAGHYRGQCHSCCNRARQERLQRRRETQV

SEQ ID NO: 150 - DNA, 474 bp, Artificial Sequence
  HPV18 E6 protein
atggcgcgctttgaggatccaacacggcgaccctacaagctacctgatctgtgcacggaactgagcacttcactgcaagacatagaaataacctgtgtatattgcaagacagtattggaacttacagaggtatttgaatttgcatttaaagatttatttgtggtgtatagagacagtataccgcatgctgcatgccataaatgtatagatttttattctagaattagagaattaagacattattcagactctgtgtatggagacacattggaaaaactaactaacactgggttatacaatttattaataaggtgcctgcggtgccagaaaccgttgaatccagcagaaaaacttagacaccttaatgaaaaacgacgatttcacaacatagctgggcactatagaggccagtgccattcgtgctgcaaccgagcacgacaggaaagactccaacgacgcagagaaacacaagta

SEQ ID NO: 151 - protein, 98 aa, Artificial Sequence
  HPV16 E7
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 152 - protein, 93 aa, Artificial Sequence
  HPV1 E7 protein
MVGEMPALKDLVLQLEPSVLDLDLYCYEEVPPDDIEEELVSPQQPYAVVASCAYCEKLVRLTVLADHSAIRQLEELLLRSLNIVCPLCTLQRQ

SEQ ID NO: 153 - DNA, 279 bp, Artificial Sequence
  HPV1 E7 protein
atggtgggcgaaatgccagcactaaaggacctggttcttcaacttgaaccaagcgtcctagatttagatctttattgttacgaggaggtgcctcctgatgacatagaggaggagttagtgtcgcctcagcaaccttatgctgtcgttgcttcctgtgcctattgcgagaaactggttcgattgaccgtcctcgcggatcacagcgccattagacagctggaggaactccttctgcgatctttgaacatcgtgtgcccactgtgcaccctacagcgacag

SEQ ID NO: 154 - protein, 92 aa, Artificial Sequence
  HPV2 E7 protein
MHGNRPSLKDITLILDEIPEIVDLHCDEQFDSSEEENNHQLTEPDVQAYGVVTTCCKCGRTVRLVVECGQADLRELEQLFLKTLTLVCPHCA

SEQ ID NO: 155 - DNA, 276 bp, Artificial Sequence
  HPV2 E7 protein
atgcacggcaaccgacccagcctcaaggacattacactaatattggatgaaatacccgaaattgttgacctacattgcgacgagcaatttgacagctcagaagaagaaaataaccatcaactgacagaaccagatgtgcaggcctacggggtggtaactacctgctgtaagtgtggcagaaccgtccggctggtggttgagtgcggacaagcagacctaagagagctggaacagctgttcttgaagacgctgactctagtgtgccctcactgcgcc

SEQ ID NO: 156 - protein, 103 aa, Artificial Sequence
  HPV5 E7 protein
MIGKEVTVQDIILELSEVQPEVLPVDLFCEEELPNEQETEEEPDIERISYKVIAPCGCRHCEVKLRIFVHATEFGIRAFQQLLTGDLQLLCPDCRGNCKHDGS

SEQ ID NO: 157 - DNA, 309 bp, Artificial Sequence
  HPV5 E7 protein
atgattggtaaagaggtcaccgtgcaagatattattctggagctcagtgaggtgcagcccgaagtgctaccagttgacctgttttgtgaagaggaattaccaaacgagcaggaaacggaggaggagcctgacatcgaaaggatctcttacaaagttatagctccgtgcggttgcaggcactgtgaggtcaagcttcgcatttttgtccacgccacagaatttggtattagagctttccaacagctattgaccggagatctgcagctcctgtgtcctgactgtcgcggaaactgcaaacatgacggatcc

SEQ ID NO: 158 - protein, 98 aa, Artificial Sequence
  HPV6 E7 protein
MHGRHVTLKDIVLDLQPPDPVGLHCYEQLVDSSEDEVDEVDGQDSQPLKQHYQIVTCCCGCDSNVRLVVQCTETDIREVQQLLLGTLNIVCPICAPKT

SEQ ID NO: 159 - DNA, 294 bp, Artificial Sequence
  HPV6 E7 protein
atgcatggaagacatgttaccctaaaggatattgtattagacctgcaacctccagaccctgtagggttacattgctatgagcaattagtagacagctcagaagatgaggtggacgaagtggacggacaagattcacaacctttaaaacaacattaccaaatagtgacctgttgctgtggatgtgacagcaacgttcgactggttgtgcagtgtacagaaacagacatcagagaagtgcaacagcttctgttgggaacactaaacatagtgtgtcccatctgcgcaccgaagaca

SEQ ID NO: 160 - protein, 103 aa, Artificial Sequence
  HPV8 E7 protein
MIGKEVTVQDFVLKLSEIQPEVLPVDLLCEEELPNEQETEEELDIERTVFKIVAPCGCSCCQVKLRLFVNATDSGIRTFQELLFRDLQLLCPECRGNCKHGGS

SEQ ID NO: 161 - protein, 98 aa, Artificial Sequence
  HPV11 E7 protein
MHGRLVTLKDIVLDLQPPDPVGLHCYEQLEDSSEDEVDKVDKQDAQPLTQHYQILTCCCGCDSNVRLVVECTDGDIRQLQDLLLGTLNIVCPICAPKP

SEQ ID NO: 162 - DNA, 294 bp, Artificial Sequence
  HPV11 E7 protein
atgcatggaagacttgttaccctaaaggatatagtactagacctgcagcctcctgaccctgtagggttacattgctatgagcaattagaagacagctcagaagatgaggtggacaaggtggacaaacaagacgcacaacctttaacacaacattaccaaatactgacctgttgctgtggatgtgacagcaacgtccgactggttgtggagtgcacagacggagacatcagacaactacaagaccttttgctgggcacactaaatattgtgtgtcccatctgcgcaccaaaacca

SEQ ID NO: 163 - protein, 105 aa, Artificial Sequence
  HPV18 E7 protein
MHGPKATLQDIVLHLEPQNEIPVDLLCHEQLSDSEEENDEIDGVNHQHLPARRAEPQRHTMLCMCCKCEARIKLVVESSADDLRAFQQLFLNTLSFVCPWCASQQ

SEQ ID NO: 164 - DNA, 315 bp, Artificial Sequence
  HPV18 E7 protein
atgcatggacctaaggcaacattgcaagacattgtattgcatttagagccccaaaatgaaattccggttgaccttctatgtcacgagcaattaagcgactcagaggaagaaaacgatgaaatagatggagttaatcatcaacatttaccagcccgacgagctgaaccacaacgtcacacaatgttgtgtatgtgttgtaagtgtgaagccagaattaagctagtagtagaaagctcagcagacgaccttcgagcattccagcagctgtttctgaacaccctgtcctttgtgtgtccgtggtgtgcatcccagcag

SEQ ID NO: 165 - DNA, 2002 bp, Artificial Sequence
  Fusion Protein STF2.HPV16 E6
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaaaaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaactacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattctgcagcaggccggtacttccgttctggcgcaggctaaccaggtcccgcagaacgtgctgagcctgttagcgcatcaaaaacgtactgctatgttccaagatccacaagagcgtccacgtaaactgccgcagctgtgcaccgaactgcagaccactatccacgatatcattctggaatgcgtgtattgcaaacaacagctgctgcgtcgtgaagtctacgattttgcctttcgcgacctgtgcatcgtttatcgcgacggtaacccgtatgctgtctgcgacaaatgcctgaaattttacagcaaaatctccgagtatcgtcattactgctattctctgtacggcacgactctggaacagcagtataacaaaccgctgtgcgacctgctgattcgttgcattaactgccagaagccgctgtgtccggaagaaaaacaacgtcacctggacaaaaagcagcgcttccacaacatccgtggtcgttggaccggccgttgcatgagctgctgccgctccagccgtacccgccgtgaaactcagctgtaatgagctgagc

SEQ ID NO: 166 - protein, 473 aa, Artificial Sequence
  HPV16 L2
MRHKRSAKRTKRASATQLYKTCKQAGTCPPDIIPKVEGKTIADQILQYGSMGVFFGGLGIGTGSGTGGRTGYIPLGTRPPTATDTLAPVRPPLTVDPVGPSDPSIVSLVEETSFIDAGAPTSVPSIPPDVSGFSITTSTDTTPAILDINNTVTTVTTHNNPTFTDPSVLQPPTPAETGGHFTLSSSTISTHNYEEIPMDTFIVSTNPNTVTSSTPIPGSRPVARLGLYSRTTQQVKVVDPAFVTTPTKLITYDNPAYEGIDVDNTLYFSSNDNSINIAPDPDFLDIVALHRPALTSRRTGIRYSRIGNKQTLRTRSGKSIGAKVHYYYDFSTIDPAEEIELQTITPSTYTTTSHAASPTSINNGLYDIYADDFITDTSTTPVPSVPSTSLSGYIPANTTIPFGGAYNIPLVSGPDIPINITDQAPSLIPIVPGSPQYTIIADAGDFYLHPSYYMLRKRRKRLPYFFSDVSLAA

SEQ ID NO: 167 - protein, 234 aa, Artificial Sequence
  HPV1 L2 protein
MVTFDNPAFEPELDEVSIIFQRDLDALAQTPVPEFRDVVYLSKPTFSREPGGRLRVSRLGKSSTIRTRLGTAIGARTHFFYDLSSIAPEDSIELLPLGEHSQTTVISSNLGDTAFIQGETAEDDLEVISLETPQLYSEEELLDTNESVGENLQLTITNSEGEVSILDLTQSRVRPPFGTEDTSLHVYYPNSSKGTPIINPEESFTPLVIIALNNSTGDFELHPSLRKRRKRAYV

SEQ ID NO: 168 - DNA, 702 bp, Artificial Sequence
  HPV1 L2 protein
atggtcacttttgataatccagcatttgagccagagcttgatgaggtgtctattatcttccaaagagacttagatgctcttgctcagacaccagtgcctgaatttagagatgtagtttatctgagcaagcccacattttcgcgggaaccagggggacggttaagggttagccgccttggcaaaagttcaactattcgtacacgcctgggcacagcaattggcgccagaacccactttttctatgatttaagttctattgctccagaagactcaattgaattattgcctttaggtgagcatagtcaaacaacagtcattagttccaacttaggtgacacagcatttatacaaggtgagacagcagaggatgacttagaagttatctctttagaaacaccacaattatattcagaagaagagcttttagacacaaacgaaagtgtgggcgaaaatttgcaacttactattactaactcagagggtgaggtttctatactagatttaacacaaagcagagtcaggccaccttttggcactgaagatactagcttgcatgtatattacccaaattcttctaaagggactccaataattaatcctgaagaatcatttacacctttggttattatagctcttaacaactcaacaggggattttgagttacatcctagtcttagaaagcgtcgtaaaagagcttatgta

SEQ ID NO: 169 - protein, 468 aa, Artificial Sequence
  HPV2 L2 protein
MSIRAKRRKRASPTDLYRTCKQAGTCPPDIIPRVEQNTLADKLLKWGSLGVFFGGLGIGTGSGTGGRTGYIPVGSRPTTVVDIGPTPRPPVIIEPVGASEPSIVTLVEDSSIINAGASHPTFTGTGGFEVTTSTVTDPAVLDITPSGTSVQVSSSSFLNPLYTEPAIVEAPQTGEVSGHVLVSTATSGSHGYEEIPMQTFATSGGSGTEPISSTPLPGVRRVAGPRLYSRANQQVQVRDPAFLARPADLVTFDNPVYDPEETIIFQHPDLHEPPDPDFLDIVALHRPALTSRRGTVRFSRLGRRATLRTRSGKQIGARVHFYHDISPIGTEELEMEPLLPPASTDNTDMLYDVYADSDVLQPLLDELPAAPRGSLSLADTAVSATSASTLRGSTTVPLSSGIDVPVYTGPDIEPPNVPGMGPLIPVAPSLPSSVYIFGGDYYLMPSYVLWPKRRKRVHYFFADGFVAA

SEQ ID NO: 170 - DNA, 1404 bp, Artificial Sequence
  HPV2 L2 protein
atgtctatacgtgccaagcgtcggaagcgcgcctcccccacagacctctatcgtacctgcaagcaggcaggtacctgccccccagacattatcccaagagtggaacagaacactttagcagataaactccttaagtggggcagtttaggtgtgttttttgggggtctaggtataggcaccggcagcggcacaggggggcgtactgggtacattcctgtaggttcgcgacccaccactgtagttgacattggtccaacgcccaggccgcctgttatcattgaacctgtgggggcctctgaaccctctattgtcactttggtggaggactctagcatcattaacgcaggagcttcacatcccacctttactggtactggtggcttcgaagtgacaacctccaccgttacagaccccgccgtcttggatatcaccccctcaggtaccagtgtgcaggtcagcagcagtagctttcttaacccactatacactgagccagctattgtggaggctccccaaacaggggaagtatctggccatgtacttgttagtacagccacctcagggtctcatggctatgaggaaataccaatgcagacgtttgccacgtcggggggcagcggtacagagcctatcagtagcacacccctccctggcgtgcggagagttgccggaccccgcctgtacagtagagccaatcagcaagtgcaagtcagggatcctgcgtttcttgcaaggcctgctgatctagtaacatttgacaatcctgtgtatgacccagaggaaactataatatttcagcatccagacttgcatgagccaccggatcctgattttttggacatagtggcgttgcatcgtcccgccctcacgtccagaaggggtactgtccgttttagtaggttgggacgcagggctacactccgcacccgtagtggtaaacaaattggggcacgggtgcacttctatcatgatattagccctataggtactgaggagttggagatggagccactgttgcccccagcttctactgataacacagatatgttatatgatgtttatgctgattcggatgtccttcagccattgcttgatgagttacccgccgcccctcgcggttcactctctctggctgacactgctgtgtctgccacctccgcatctacactacgggggtccactactgtccctttatcaagtggtattgatgtgcctgtgtacactggtcctgacattgaaccacccaatgttcctggcatgggacctctgattcctgtggctccatccttaccatcgtctgtgtacatatttgggggagattattatttgatgccaagttatgtcttgtggcctaaacgacgtaaacgtgtccactatttctttgcagatggctttgtggcggcc

SEQ ID NO: 171 - protein, 518 aa, Artificial Sequence
  HPV5 L2 protein
MARAKRVKRDSVTHIYQTCKQAGTCPPDVINKVEQTTVADNILKYGSAGVFFGGLGISTGRGTGGATGYVPLGEGPGVRVGGTPTVVRPSLVPETIGPVDILPIDTVNPVEPTASSVVPLTESTGADLLPGEVETIAEIHPVPEGPSVDTPVVTTSTGSSAVLEVAPEPIPPTRVRVSRTQYHNPSFQIITESTPAQGESSLADHVLVTSGSGGQRIGGDITDIIELEEIPSRYTFEIEEPTPPRRSSTPLPRNQSVGRRRGFSLTNRRLVQQVQVDNPLFLTQPSKLVRFAFDNPVFEEEVTNIFENDLDVFEEPPDRDFLDVRELGRPQYSTTPAGYVRVSRLGTRATIRTRSGAQIGSQVHFYRDLSSINTEDPIELQLLGQHSGDATIVQGPVESTFIDMDISENPLSESIEAYSHDLLLDETVEDFSGSQLVIGNRRSTNSYTVPRFETTRNGSYYTQDTKGYYVAYPESRNNAEIIYPTPDIPVVIIHPHDSTGDFYLHPSLRRRKRKRKYL

SEQ ID NO: 172 - DNA, 1554 bp, Artificial Sequence
  HPV5 L2 protein
atggcgcgtgcaaaaagggtcaagcgagactctgtaactcatatttaccaaacctgcaaacaggcaggcacttgcccccctgatgttattaataaagtggaacaaacaacagttgctgacaatattttaaaatatggcagtgctggtgtattttttggtggccttggtattagtacaggccgaggaactgggggtgctacagggtacgtgccacttggggaaggtcctggtgtccgtgtcggaggaacccccacggttgtaaggccttccttggttcctgaaacaatcgggcccgttgatattttgcccattgatacagttaaccccgtggaacctacagcatcatccgtggtccctctaactgagtccacaggcgctgatttacttccaggtgaagtagaaacaattgctgaaatccatcctgtacctgaggggccatcagtggatacccctgtagttaccactagcacaggttccagtgctgttttagaggttgccccagagcctattcctccaacacgggtcagggtttcacgcacacagtatcacaatccatcttttcaaataataactgagtctactccagcacaaggggaatcgtctcttgcagatcacgttttggtgacatcgggttctggggggcaacgaatagggggtgatataactgacataattgagttagaggaaattcctagtaggtatacatttgaaattgaagaaccaactcctccacgccgcagcagtactccattgccacgcaatcaatctgtaggccgtaggaggggtttctctttgactaatagacgtttagtacagcaggtacaagtggacaatccattgtttctaactcaaccatctaagttagttcgttttgcatttgataatcctgtttttgaggaagaagtgactaatatatttgaaaatgatctggatgtctttgaagaacctccagacagagattttcttgatgttagggaattgggacgtccacaatattctacaacaccagcgggatatgttagagtaagcaggttggggactcgagccactattcgcactcgctctggtgcacagatagggtcgcaagtccatttttacagagatcttagctctattaatactgaagatcctattgaattacaattattaggccaacattcaggtgatgctactatagtccagggacctgttgaaagcacatttatagatatggatatttctgaaaatccattatctgaaagcattgaagcatattcacatgatttattattagatgaaacggtggaagatttcagtgggtctcagctggttataggtaatcgaaggagcacaaactcttacactgttcctaggtttgaaactacaagaaatggttcatactatacacaagacacaaagggatattatgttgcatatccagagtcacgtaataatgcagaaatcatttatcctacacctgatattcctgtagtcattatacaccctcatgacagtacaggggacttttatttacatcccagtcttcgcaggcgcaaacgtaaaagaaaatatttg

SEQ ID NO: 173 - protein, 459 aa, Artificial Sequence
  HPV6 L2 protein
MAHSRARRRKRASATQLYQTCKLTGTCPPDVIPKVEHNTIADQILKWGSLGVFFGGLGIGTGSGTGGRTGYVPLGTSAKPSITSGPMARPPVVVEPVAPSDPSIVSLIEESAIINAGAPEIVPPAHGGFTITSSETTTPAILDVSVTSHTTTSIFRNPVFTEPSVTQPQPPVEANGHILISAPTITSHPIEEIPLDTFVISSSDSGPTSSTPVPGTAPRPRVGLYSRALHQVQVTDPAFLSTPQRLITYDNPVYEGEDVSVQFSHDSIHNAPDEAFMDIIRLHRPAIASRRGLVRYSRIGQRGSMHTRSGKHIGARIHYFYDISPIAQAAEEIEMHPLVAAQEDTFDIYAESFEPDINPTQHPVTNISDTYLTSTPNTVTQPWGNTTVPLSIPNDLFLQSGPDITFPTAPMGTPFSPVTPALPTGPVFITGSGFYLHPAWYFARKRRKRIPLFFSDVAA

SEQ ID NO: 174 - DNA, 1377 bp, Artificial Sequence
  HPV6 L2 protein
atggcacatagtagggcccgacgacgcaagcgtgcgtcagctacacagctatatcaaacatgtaaactcactggaacatgccccccagatgtaattcctaaggtggaacacaacaccattgcagatcaaatattaaaatgggggagtttgggggtgttttttggagggttgggtataggcaccggttccggcactgggggtcgtactggctatgttcccttaggaacttctgcaaaaccttctattactagtgggcctatggctcgtcctcctgtggtggtggagcctgtggccccttcggatccatccattgtgtctttaattgaagaatcggcaatcattaacgcaggggcgcctgaaattgtgccccctgcacacggtgggtttacaattacatcctctgaaacaactacccctgcaatattggatgtatcagttactagtcatactactactagtatatttagaaatcctgtctttacagaaccttctgtaacacaaccccaaccacccgtggaggctaatggacatatattaatttctgcacccactataacgtcacaccctatagaggaaattcctttagatacttttgtgatatcctctagtgatagcggtcctacatccagtacccctgttcctggtactgcacctcggcctcgtgtgggcctatatagtcgtgcattgcaccaggtgcaggttacagaccctgcatttctttccactcctcaacgcttaattacatatgataaccctgtatatgaaggggaggatgttagtgtacaatttagtcatgattctatacacaatgcacctgatgaggcttttatggacataattcgtttgcacagacctgctattgcgtcccgacgtggccttgtgcggtacagtcgcattggacaacgggggtctatgcacactcgcagcggaaagcacataggggcccgcattcattatttttatgatatttcacctattgcacaagctgcagaagaaatagaaatgcaccctcttgtggctgcacaggaagatacatttgatatttatgctgaatcttttgaacctgacattaaccctacccaacaccctgttacaaatatatcagatacatatttaacttccacacctaatacagttacacaaccgtggggtaacaccacagttccattgtcaattcctaatgacctgtttttacagtctggccctgatataacttttcctactgcacctatgggaacaccctttagtcctgtaactcctgctttacctacaggccctgttttcattacaggttctggattttatttgcatcctgcatggtattttgcacgtaaacgccgtaaacgtattcccttatttttttcagatgtggcggcc

SEQ ID NO: 175 - protein, 518 aa, Artificial Sequence
  HPV8 L2 protein
MARARRVKRDSVTHIYQTCKQAGTCPPDVINKVEQTTVADNILKYGSAGVFFGGLGIGTGRGTGGVTGYTPLSEGPGIRVGNTPTVVRPSLVPEAVGPMDILPIDTIDPVEPSVSSVVPLTESSGADLLPGEVETIAEIHPVPEGPTIDSPVVTTSKGSSAILEVAPEPTPPTRVRVSRTQYHNPSFQIITDSTPTQGESSLADHILVTSGSGGQTIGSDITDVIELQEFPSRYSFEIDEPTPPRQSSTPIERPQVVGRRRGISLTNRRLIQQVAVEDPLFLSKPSKLVRFSFDNPVFEEEVTNIFEQDVDMVEEPPDRDFLDVRQLGRPQYSTTPAGYVRVSRLGTRGTIRTRSGAQIGSQVHFYRDLSSINTEDPIELQLLGQHSGDSTIVQGPVESTFVNVDISENPLSESIQAFSDDLLLDETVEDFSGSQLVIGNRRSTTSYTVPRFETTRSGSYYVQDTKGYYVAYPESRNNEEIIYPTPDLPVVIIHTHDNSGDFFLHPSLRRRKRKRKYL

SEQ ID NO: 176 - protein, 455 aa, Artificial Sequence
  HPV11 L2 protein
MKPRARRRKRASATQLYQTCKATGTCPPDVIPKVEHTTIADQILKWGSLGVFFGGLGIGTGAGSGGRAGYIPLGSSPKPAITGGPAARPPVLVEPVAPSDPSIVSLIEESAIINAGAPEVVPPTQGGFTITSSESTTPAILDVSVTNHTTTSVFQNPLFTEPSVIQPQPPVEASGHILISAPTITSQHVEDIPLDTFVVSSSDSGPTSSTPLPRAFPRPRVGLYSRALQQVQVTDPAFLSTPQRLVTYDNPVYEGEDVSLQFTHESIHNAPDEAFMDIIRLHRPAITSRRGLVRFSRIGQRGSMYTRSGQHIGARIHYFQDISPVTQAAEEIELHPLVAAENDTFDIYAEPFDPIPDPVQHSVTQSYLTSTPNTLSQSWGNTTVPLSIPSDWFVQSGPDITFPTASMGTPFSPVTPALPTGPVFITGSDFYLHPTWYFARRRRKRIPLFFTDVAA

SEQ ID NO: 177 - DNA, 1365 bp, Artificial Sequence
  HPV11 L2 protein
atgaaacctagggcacgcagacgtaaacgtgcgtcagccacacaactatatcaaacatgcaaggccactggtacatgtcccccagatgtaattcctaaagttgaacatactactattgcagatcaaatattaaaatggggaagcttaggggttttttttggtgggttaggtattggtacaggggctggtagtggcggtcgtgcagggtatatacccttgggaagctctcccaagcctgctattactggggggccagcagcacgtccgccagtgcttgtggagcctgttgccccttccgatccctccattgtgtccttaattgaggagtctgctattattaatgctggtgcacctgaggtggtaccccctacacagggtggctttactataacatcatctgaatcgactacacctgctattttagatgtgtctgttaccaatcacactaccactagtgtgtttcaaaatcccctgtttacagaaccgtctgtaatacagccccaaccacctgtggaggccagtggtcacatacttatatctgccccaacaataacatcccaacatgtagaagacattccactagacacttttgttgtatcctctagtgatagtggacctacatccagtactcctcttcctcgtgcttttcctcggcctcgggtgggtttgtatagtcgtgccttacagcaggtacaggttacggaccccgcgtttttgtccacgccacagcgattggtaacttatgacaaccctgtctatgaaggagaagatgtaagtttacaatttacccatgagtctatccacaatgcacctgatgaagcatttatggatattattagactacatagaccagctataacgtccagacggggtcttgtgcgttttagtcgcattgggcaacgggggtccatgtacacacgcagtggacaacatataggtgcccgcatacattattttcaggacatttcaccagttacacaagctgcagaggaaatagaactgcaccctctagtggctgcagaaaatgacacgtttgatatttatgctgaaccatttgaccctatccctgaccctgtccaacattctgttacacagtcttatcttacctccacacctaataccctttcacaatcgtggggtaataccacagtcccattgtcaatccctagtgactggtttgtgcagtctgggcctgacataacttttcctactgcatctatgggaacaccctttagtcctgtaactcctgctttacctacaggccctgtttttattacaggttctgacttctatttgcatcctacatggtactttgcacgcagacgccgtaaacgtattcccttattttttacagatgtggcggcc

SEQ ID NO: 178 - protein, 462 aa, Artificial Sequence
  HPV18 L2 protein
MVSHRAARRKRASVTDLYKTCKQSGTCPPDVVPKVEGTTLADKILQWSSLGIFLGGLGIGTGSGTGGRTGYIPLGGRSNTVVDVGPTRPPVVIEPVGPTDPSIVTLIEDSSVVTSGAPRPTFTGTSGFDITSAGTTTPAVLDITPSSTSVSISTTNFTNPAFSDPSIIEVPQTGEVAGNVFVGTPTSGTHGYEEIPLQTFASSGTGEEPISSTPLPTVRRVAGPRLYSRAYQQVSVANPEFLTRPSSLITYDNPAFEPVDTTLTFDPRSDVPDSDFMDIIRLHRPALTSRRGTVRFSRLGQRATMFTRSGTQIGARVHFYHDISPIAPSPEYIELQPLVSATEDNDLFDIYADDMDPAVPVPSRSTTSFAFFKYSPTISSASSYSNVTVPLTSSWDVPVYTGPDITLPSTTSVWPIVSPTAPASTQYIGIHGTHYYLWPLYYFIPKKRKRVPYFFADGFVAA

SEQ ID NO: 179 - DNA, 1386 bp, Artificial Sequence
  HPV18 L2 protein
atggtatcccaccgtgccgcacgacgcaaacgggcttcggtaactgacttatataaaacatgtaaacaatctggtacatgtccacctgatgttgttcctaaggtggagggcaccacgttagcagataaaatattgcaatggtcaagccttggtatatttttgggtggacttggcataggtactggcagtggtacagggggtcgtacagggtacattccattgggtgggcgttccaatacagtggtggatgttggtcctacacgtcccccagtggttattgaacctgtgggccccacagacccatctattgttacattaatagaggactccagtgtggttacatcaggtgcacctaggcctacgtttactggcacgtctgggtttgatataacatctgcgggtacaactacacctgcggttttggatatcacaccttcgtctacctctgtgtctatttccacaaccaattttaccaatcctgcattttctgatccgtccattattgaagttccacaaactggggaggtggcaggtaatgtatttgttggtacccctacatctggaacacatgggtatgaggaaatacctttacaaacatttgcttcttctggtacgggggaggaacccattagtagtaccccattgcctactgtgcggcgtgtagcaggtccccgcctttacagtagggcctaccaacaagtgtcagtggctaaccctgagtttcttacacgtccatcctctttaattacatatgacaacccggcctttgagcctgtggacactacattaacatttgatcctcgtagtgatgttcctgattcagattttatggatattatccgtctacataggcctgctttaacatccaggcgtgggactgttcgctttagtagattaggtcaacgggcaactatgtttacccgcagcggtacacaaataggtgctagggttcacttttatcatgatataagtcctattgcaccttccccagaatatattgaactgcagcctttagtatctgccacggaggacaatgacttgtttgatatatatgcagatgacatggaccctgcagtgcctgtaccatcgcgttctactacctcctttgcattttttaaatattcgcccactatatcttctgcctcttcctatagtaatgtaacggtccctttaacctcctcttgggatgtgcctgtatacacgggtcctgatattacattaccatctactacctctgtatggcccattgtatcacccacggcccctgcctctacacagtatattggtatacatggtacacattattatttgtggccattatattattttattcctaagaaacgtaaacgtgttccctatttttttgcagatggctttgtggcggcc

SEQ ID NO: 180 - protein, 494 aa, Artificial Sequence
  HPV2 L1 protein
MALWRPNESKVYLPPTPVSKVISTDVYVTRTNVYYHGGSSRLLTVGHPYYSIKKSNNKVAVPKVSGYQYRVFHVKLPDPNKFGLPDADLYDPDTQRLLWACVGVEVGRGQPLGVGVSGHPYYNRLDDTENAHTPDTADDGRENISMDYKQTQLFILGCKPPIGEHWSKGTTCNGSSAAGDCPPLQFTNTTIEDGDMVETGFGALDFATLQSNKSDVPLDICTNTCKYPDYLKMAAEPYGDSMFFSLRREQMFTRHFFNLGGKMGDTIPDELYIKSTSVPTPGSHVYTSTPSGSMVSSEQQLFNKPYWLRRAQGHNNGMCWGNRVFLTVVDTTRSTNVSLCATEASDTNYKATNFKEYLRHMEEYDLQFIFQLCKITLTPEIMAYIHNMDPQLLEDWNFGVPPPPSASLQDTYRYLQSQAITCQKPTPPKTPTDPYASLTFWDVDLSESFSMDLDQFPLGRKFLLQRGAMPTVSRKRAAVSGTTPPTSKRKRVRR

SEQ ID NO: 181 - DNA, 1482 bp, Artificial Sequence
  HPV2 L1 protein
atggctttgtggcggcctaatgaaagcaaggtatacctacctccaacacctgtttcaaaggtgatcagtacggatgtctatgtcacgcggactaatgtttattaccatggtggcagttctaggcttctcactgtgggtcatccatattactctataaagaagagtaataataaggtggctgtgcccaaggtatctggctaccaatatcgtgtatttcacgtgaagttgccagatcccaataagtttggcctgcccgatgctgatttgtatgatccggatacccagagacttctgtgggcgtgcgtgggagtagaggtgggccgtgggcagcctttgggtgtgggtgtgtctggtcacccatattacaatagactggatgacactgaaaatgcacacacacctgatacagctgatgatggcagggaaaacatttctatggattataaacagacacagctgttcattctgggctgcaaaccccctattggtgagcactggtctaagggtaccacctgtaatgggtcttctgcggctggtgactgcccgcccctccaatttactaacacaactattgaggacggggatatggttgaaacagggttcggtgccttggattttgccactctgcagtcaaataagtcagatgttcctttggatatttgtaccaatacctgtaaatatcctgattatctgaagatggctgcagagccttatggtgattctatgttcttctcgctgcgtagggaacaaatgttcactcgtcattttttcaatctgggtggtaagatgggtgacaccatcccggatgagttatacattaaaagtacctcagttccaactccaggcagtcatgtttatacttccactcctagtggctctatggtgtcctctgaacaacagttgtttaataagccttactggctacggagggcccaagggcacaacaatggtatgtgctggggcaatagggtctttctgactgtggtggacaccacacgtagcactaatgtatctctgtgtgccactgaggcgtctgatactaattataaggctaccaattttaaggaatatctcaggcatatggaggaatatgatttgcagttcatcttccaactgtgcaagataacccttactcctgaaattatggcctatatacataatatggatccccagttgttagaggattggaacttcggtgtaccccctccgccgtctgccagtttacaggatacctatagatatttgcagtcccaggctattacatgtcaaaaacctacacctcctaagacccctaccgatccctatgcctccctgaccttttgggatgtggatctcagtgaaagtttttccatggatctggaccaatttcccttgggtcgcaagtttttgctgcagcggggggctatgcctaccgtgtctcgtaagcgcgccgctgtttcggggaccacgccgcccactagtaaacgaaaacgggtaaggcgt

SEQ ID NO: 182 - protein, 516 aa, Artificial Sequence
  HPV5 L1 protein
MAVWHSANGKVYLPPSTPVARVQSTDEYIQRTNIYYHAFSDRLLTVGHPYFNVYNINGDKLEVPKVSGNQHRVFRLKLPDPNRFALADMSVYNPDKERLVWACRGLEIGRGQPLGVGSTGHPYFNKVKDTENSNAYITFSKDDRQDTSFDPKQIQMFIVGCTPCIGEHWDKAVPCAENDQQTGLCPPIELKNTYIEDGDMADIGFGNMNFKALQDSRSDVSLDIVNETCKYPDFLKMQNDIYGDACFFYARREQCYARHFFVRGGKTGDDIPGAQIDNGTYKNQFYIPGADGQAQKTIGNSMYFPTVSGSLVSSDAQLFNRPFWLQRAQGHNNGILWANQMFITVVDNTRNTNFSISVYNQAGALKDVADYNADQFREYQRHVEEYEISLILQLCKVPLKAEVLAQINAMNSSLLEDWQLGFVPTPDNPIQDTYRYIDSLATRCPDKNPPKEKEDPYKGLHFWDVDLTERLSLDLDQYSLGRKFLFQAGLQQTTVNGTKAVSYKGSNRGTKRKRKN

SEQ ID NO: 183 - DNA, 1548 bp, Artificial Sequence
  HPV5 L1 protein
atggcagtgtggcactcggctaatggtaaagtatatcttccaccatcgacaccggtggccagagtccaaagcaccgatgaatacattcaaagaacaaatatctactatcatgcatttagtgacagattgttaactgtaggtcatccttatttcaatgtatacaatattaatggtgataagcttgaggttcctaaggtttcaggaaatcaacacagagtatttcgcctaaaattaccagatcctaacagatttgcattagctgatatgtctgtttacaaccctgacaaagaacgtttggtttgggcctgtagaggcttagaaataggtaggggccagccattaggtgtagggagtactggtcacccttatttcaataaagtaaaagatacagaaaacagtaatgcatacataacattttctaaagatgacagacaggatacatcttttgatcctaaacagatccaaatgtttattgtaggatgcacaccttgcataggagagcattgggataaagctgttccatgtgcagaaaatgatcagcaaactggcctttgtcctcctattgaactaaaaaacacatatatagaagatggtgatatggcagacataggttttgggaacatgaattttaaggcacttcaagatagtagatcagatgtcagtttagacatcgtcaatgaaacttgcaagtatccagattttttaaagatgcaaaacgatatttatggcgatgcgtgctttttttatgctcgtagggagcaatgttatgccagacacttttttgttagagggggaaaaactggtgatgacattccaggtgcacaaattgacaatggtacatacaaaaatcagttttacattccaggggctgatggccaagctcaaaagactataggaaattccatgtatttcccaactgttagtggctcattagtatccagtgatgctcaattgtttaacaggcccttctggctccaaagagcccaaggtcataataatggcatcctgtgggctaatcaaatgtttatcacagtggttgacaacacaagaaatactaatttcagtatttctgtatataatcaggctggagcactaaaagatgttgcagactataatgcagatcaatttagagaatatcaaagacatgtagaagaatatgaaatatctttaattctacaactctgtaaggttcctttaaaggcagaggtattggcacagatcaatgcaatgaactcttcgttattggaggattggcagttaggatttgttcccactcctgataatccaattcaggacacctacagatatattgactctttggctacacggtgtccagataagaatcctccgaaagaaaaggaagacccttataagggcttacatttttgggatgtagatttaactgaaagattgtcattagatttagatcaatattccttaggcagaaaatttttattccaagctgggttacaacaaacgaccgttaacggtacaaaagcagtgtcttataaagggtctaatagaggaacaaaacgcaaacgtaaaaat

SEQ ID NO: 184 - protein, 500 aa, Artificial Sequence
  HPV6 L1 protein
MWRPSDSTVYVPPPNPVSKVVATDAYVTRTNIFYHASSSRLLAVGHPYFSIKRANKTVVPKVSGYQYRVFKVVLPDPNKFALPDSSLFDPTTQRLVWACTGLEVGRGQPLGVGVSGHPFLNKYDDVENSGSGGNPGQDNRVNVGMDYKQTQLCMVGCAPPLGEHWGKGKQCTNTPVQAGDCPPLELITSVIQDGDMVDTGFGAMNFADLQTNKSDVPIDICGTTCKYPDYLQMAADPYGDRLFFFLRKEQMFARHFFNRAGEVGEPVPDTLIIKGSGNRTSVGSSIYVNTPSGSLVSSEAQLFNKPYWLQKAQGHNNGICWGNQLFVTVVDTTRSTNMTLCASVTTSSTYTNSDYKEYMRHVEEYDLQFIFQLCSITLSAEVMAYIHTMNPSVLEDWNFGLSPPPNGTLEDTYRYVQSQAITCQKPTPEKQKPDPYKNLSFWEVNLKEKFSSELDQYPLGRKFLLQSGYRGRSSIRTGVKRPAVSKASAAPKRKRAKTKR

SEQ ID NO: 185 - DNA, 1500 bp, Artificial Sequence
  HPV6 L1 protein
atgtggcggcctagcgacagcacagtatatgtgcctcctcctaaccctgtatccaaagttgttgccacggatgcttatgttactcgcaccaacatattttatcatgccagcagttctagacttcttgcagtgggtcatccttatttttccataaaacgggctaacaaaactgttgtgccaaaggtgtcaggatatcaatacagggtatttaaggtggtgttaccagatcctaacaaatttgcattgcctgactcgtctctttttgatcccacaacacaacgtttggtatgggcatgcacaggcctagaggtgggcaggggacagccattaggtgtgggtgtaagtggacatcctttcctaaataaatatgatgatgttgaaaattcagggagtggtggtaaccctggacaggataacagggttaatgttggtatggattataaacaaacacaattatgcatggttggatgtgccccccctttgggcgagcattggggtaaaggtaaacagtgtactaatacacctgtacaggctggtgactgcccgcccttagaacttattaccagtgttatacaggatggcgatatggttgacacaggctttggtgctatgaattttgctgatttgcagaccaataaatcagatgttcctattgacatatgtggcactacatgtaaatatccagattatttacaaatggctgcagacccatatggtgatagattatttttttttctacggaaggaacaaatgtttgccagacatttttttaacagggctggcgaggtgggggaacctgtgcctgatactcttataattaagggtagtggaaatcgaacgtctgtagggagtagtatatatgttaacaccccaagcggctctttggtgtcctctgaggcacaattgtttaataagccatattggctacaaaaagcccagggacataacaatggtatttgttggggtaatcaactgtttgttactgtggtagataccacacgcagtaccaacatgacattatgtgcatccgtaactacatcttccacatacaccaattctgattataaagagtacatgcgtcatgtggaagagtatgatttacaatttatttttcaattatgtagcattacattgtctgctgaagtaatggcctatattcacacaatgaatccctctgttttggaagactggaactttgggttatcgcctcccccaaatggtacattagaagatacctataggtatgtgcagtcacaggccattacctgtcaaaagcccactcctgaaaagcaaaagccagatccctataagaaccttagtttttgggaggttaatttaaaagaaaagttttctagtgaattggatcagtatcctttgggacgcaagtttttgttacaaagtggatataggggacggtcctctattcgtaccggtgttaagcgccctgctgtttccaaagcctctgctgcccctaaacgtaagcgcgccaaaactaaaagg

SEQ ID NO: 186 - protein, 514 aa, Artificial Sequence
  HPV8 L1 protein
MAVWQSATGKVYLPPSTPVARVQSTDEYIQRTNIYYHANTDRLLTVGHPYFNVYNNNGDTLQVPKVSGNQHRVFRLKLPDPNRFALADMSVYNPDKERLVWACRGLEISRGQPLGVGSTGHPYFNKVKDTENSNSYTTTSTDDRQNTSFDPKQIQMFIVGCTPCIGEHWEKAIPCAEDQQQGLCPPIELKNTVIEDGDMADIGFGNMNFKTLQQNRSDVSLDIVNEICKYPDFLKMQNDVYGDACFFYARREQCYARHFFVRGGKTGDDIPAAQIDDGMMKNQYYIPGGQDQSQKDIGNAMYFPTVSGSLVSSDAQLFNRPFWLQRAQGHNNGILWANQMFVTVVDNTRNTNFSISVYTENGELKNITDYKSTQFREYLRHVEEYEISLILQLCKIPLKADVLAQINAMNSSLLEEWQLGFVPTPDTPIHDTYRYIDSLATRCPDKSPPKEKPDPYAKFNFWNVDLTERLSLDLDQYSLGRKFLFQAGLQQTTVNGTKSISRGSVRGTKRKRKN

SEQ ID NO: 187 - protein, 501 aa, Artificial Sequence
  HPV11 L1 protein
  VARIANT 486: Xaa = Any Amino Acid
MWRPSDSTVYVPPPNPVSKVVATDAYVKRTNIFYHASSSRLLAVGHPYYSIKKVNKTVVPKVSGYQYRVFKVVLPDPNKFALPDSSLFDPTTQRLVWACTGLEVGRGQPLGVGVSGHPLLNKYDDVENSGGYGGNPGQDNRVNVGMDYKQTQLCMVGCAPPLGEHWGKGTQCSNTSVQNGDCPPLELITSVIQDGDMVDTGFGAMNFADLQTNKSDVPLDICGTVCKYPDYLQMAADPYGDRLFFYLRKEQMFARHFFNRAGTVGEPVPDDLLVKGGNNRSSVASSIYVHTPSGSLVSSEAQLFNKPYWLQKAQGHNNGICWGNHLFVTVVDTTRSTNMTLCASVSKSATYTNSDYKEYMRHVKEFDLQFIFQLCSITLSAEVMAYIHTMNPSVLEDWNFGLSPPPNGTLEDTYRYVQSQAITCQKPTPEKEKQDPYKDMSFWEVNLKEKFSYELDQFPLGRKFLLQSGYRGRTSARTGIKRPAVXKPSTAPKRKRTKTRK

SEQ ID NO: 188 - protein, 266 aa, Artificial Sequence
  HPV16 L1 protein
NAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWN

SEQ ID NO: 189 - DNA, 798 bp, Artificial Sequence
  HPV16 L1 protein
aatgcaggtgtggataatagagaatgtatatctatggattacaaacaaacacaattgtgtttaattggttgcaaaccacctataggggaacactggggcaaaggatccccatgtaccaatgttgcagtaaatccaggtgattgtccaccattagagttaataaacacagttattcaggatggtgatatggttgatactggctttggtgctatggactttactacattacaggctaacaaaagtgaagttccactggatatttgtacatctatttgcaaatatccagattatattaaaatggtgtcagaaccatatggcgacagcttatttttttatttacgaagggaacaaatgtttgttagacatttatttaatagggctggtgctgttggtgaaaatgtaccagacgatttatacattaaaggctctgggtctactgcaaatttagccagttcaaattattttcctacacctagtggttctatggttacctctgatgcccaaatattcaataaaccttattggttacaacgagcacagggccacaataatggcatttgttggggtaaccaactatttgttactgttgttgatactacacgcagtacaaatatgtcattatgtgctgccatatctacttcagaaactacatataaaaatactaactttaaggagtacctacgacatggggaggaatatgatttacagtttatttttcaactgtgcaaaataaccttaactgcagacgttatgacatacatacattctatgaattccactattttggaggactggaat

SEQ ID NO: 190 - protein, 535 aa, Artificial Sequence
  HPV18 L1 protein
HYHLLPLYGPLYHPQPLPLHSILVYMVHIIICGHYIILFLKSVNVFPIFLQMALWRPSDNTVYLPPPSVARVVNTDDYVTRTSIFYHAGSSRLLTVGNPYFRVPASGGNKQDIPKVSAYQYRVFRVQLPDPNKFGLPDNSIYNPETQRLVWACAGVEIGRGQPLGVGLSGHPFYNKLDDTESSHAATSNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPQSLYIKGTGMRASPGSCVYSPSPSGSIVTSDSQLFNKPYWLHKAQGHNNGICWHNQLFVTVVDTTRSTNLTICASTQSPVPGQYDATKFKQYSRHVEEYDLQFIFQLCTITLTADVMSYIHSMNSSILEDWNFGVPPPPTTSLVDTYRFVQSVAITCQKDAAPAENKDPYDRLKFWNVDLKEKFSLDLDQYPLGRKFLVQAGLRRKPTIGPR

SEQ ID NO: 191 - DNA, 1315 bp, Artificial Sequence
  Fusion Protein STF2delta HPV16 E6
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcatggagcgccggtggatcctgctagcccatggaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttagcgcatcaaaaacgtactgctatgttccaagatccacaagagcgtccacgtaaactgccgcagctgtgcaccgaactgcagaccactatccacgatatcattctggaatgcgtgtattgcaaacaacagctgctgcgtcgtgaagtctacgattttgcctttcgcgacctgtgcatcgtttatcgcgacggtaacccgtatgctgtctgcgacaaatgcctgaaattttacagcaaaatctccgagtatcgtcattactgctattctctgtacggcacgactctggaacagcagtataacaaaccgctgtgcgacctgctgattcgttgcattaactgccagaagccgctgtgtccggaagaaaaacaacgtcacctggacaaaaagcagcgcttccacaacatccgtggtcgttggaccggccgttgcatgagctgctgccgctccagccgtacccgccgtgaaactcagctgtaatgagctgagc

SEQ ID NO: 192 - protein, 4 aa, Artificial Sequence
  Zinc Binding Sequence
  VARIANT 2, 3: Xaa = Any Amino Acid
CXXC

SEQ ID NO: 193 - protein, 506 aa, Artificial Sequence; identical to SEQ ID NO: 98
  Flagellin STF2

SEQ ID NO: 194 - DNA, 1518 bp, Artificial Sequence
  Flagellin STF2
atggcccaggttatcaataccaactccctgtcgttgctcacccaaaataaccttaataaaagccagagcgcactgggaaccgccatagaacgcctctcaagcggcctccggatcaattctgcaaaagacgacgccgccggtcaggccatcgcaaaccgctttaccgccaatatcaagggactgacgcaggcttcgaggaatgctaacgatggaataagcatcgctcaaaccacggagggcgccctgaacgagatcaacaacaacctacagcgcgtcagggagctcgcagtgcagtccgccaattcgaccaactcgcagtcggacctggactcgatccaagccgaaatcacccagcgcctgaatgagattgaccgggtgagcggtcagacacagtttaacggcgtgaaggtacttgcacaggataacacacttacgatacaggtgggcgccaacgatggtgaaaccatagacattgatctcaaacagattaacagccagacgctcgggttggatagcctgaatgtgcaaaaggcgtacgacgtgaaagacacggcggtcactaccaaagcctacgctaacaatggcactaccttggatgtgagcggattggatgatgcagcaatcaaggctgctaccggcggtacgaacggaaccgcgtccgtgaccggcggtgccgtgaagttcgatgctgacaacaataagtatttcgtcaccattggaggctttactggcgccgacgcagcaaagaacggcgactatgaagtgaacgtggcaaccgatggaaccgtgacgctggccgctggtgccaccaagaccaccatgccagccggcgccacaactaagaccgaggtgcaggagttaaaggacacccccgcggtggttagcgcagatgccaaaaacgcgttgatcgccggcggagtggatgcaactgatgctaatggtgcggagctggttaaaatgtcgtatacagacaagaatggtaagacgatcgagggcggttatgcccttaaggcaggagataagtattacgctgctgattacgatgaggcgacgggagctattaaggccaagacaacgtcatacacggcggcggacggaacgactaagacggctgccaatcagttgggaggggttgacgggaagacagaggtcgttacgatcgatggcaagacatacaacgcctccaaggccgctggccacgatttcaaagctcaacccgaactggccgaggccgcggcgaaaacaactgagaacccgttgcagaagattgatgcggccctggcgcaagtagatgccctgcgctcagacctgggcgccgttcaaaatcgattcaattccgcgattacaaacctgggcaatacagtaaacaatctatccgaggccagatcccgcattgaagactccgactacgcgacagaagtaagtaacatgagtcgtgcccagattctgcagcaggccggcactagtgtcctggcccaggccaatcaagtcccgcagaatgtgctgagcctactacga

SEQ ID NO: 195 - protein, 4 aa, Artificial Sequence
  TLR agonist
TIAL

SEQ ID NO: 196 - protein, 4 aa, Artificial Sequence
  TLR agonist
TTLD

SEQ ID NO: 197 - protein, 7 aa, Artificial Sequence
  TLR agonist
GTDQKID

SEQ ID NO: 198 - protein, 6 aa, Artificial Sequence
  TLR agonist
NGEVTL

SEQ ID NO: 199 - protein, 5 aa, Artificial Sequence
  TLR agonist
GADAA

SEQ ID NO: 200 - protein, 4 aa, Artificial Sequence
  TLR agonist
AGGA

SEQ ID NO: 201 - protein, 4 aa, Artificial Sequence
  TLR agonist
PATA

SEQ ID NO: 202 - protein, 4 aa, Artificial Sequence
  TLR agonist
AAGA

SEQ ID NO: 203 - protein, 4 aa, Artificial Sequence
  TLR agonist
ATTK

SEQ ID NO: 204 - protein, 12 aa, Artificial Sequence
  TLR agonist
AGATKTTMPAGA

SEQ ID NO: 205 - protein, 5 aa, Artificial Sequence
  TLR agonist
VTGTG

SEQ ID NO: 206 - protein, 10 aa, Artificial Sequence
  TLR agonist
TEAKAALTAA

SEQ ID NO: 207 - protein, 11 aa, Artificial Sequence
  TLR agonist
ASVVKMSYTDN

SEQ ID NO: 208 - protein, 603 aa, Artificial Sequence
  Fusion Protein STF2. E7
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLAHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 209 - protein, 663 aa, Artificial Sequence
  Fusion Protein STF2. E7
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLAHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 210 - protein, 760 aa, Artificial Sequence
  Fusion Protein STF2. E6E7
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLAHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQLHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 211 - protein, 978 aa, Artificial Sequence
  Fusion Protein STF2. L2
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLARHKRSAKRTKRASATQLYKTCKQAGTCPPDIIPKVEGKTIADQILQYGSMGVFFGGLGIGTGSGTGGRTGYIPLGTRPPTATDTLAPVRPPLTVDPVGPSDPSIVSLVEETSFIDAGAPTSVPSIPPDVSGFSITTSTDTTPAILDINNTVTTVTTHNNPTFTDPSVLQPPTPAETGGHFTLSSSTISTHNYEEIPMDTFIVSTNPNTVTSSTPIPGSRPVARLGLYSRTTQQVKVVDPAFVTTPTKLITYDNPAYEGIDVDNTLYFSSNDNSINIAPDPDFLDIVALHRPALTSRRTGIRYSRIGNKQTLRTRSGKSIGAKVHYYYDFSTIDPAEEIELQTITPSTYTTTSHAASPTSINNGLYDIYADDFITDTSTTPVPSVPSTSLSGYIPANTTIPFGGAYNIPLVSGPDIPINITDQAPSLIPIVPGSPQYTIIADAGDFYLHPSYYMLRKRRKRLPYFFSDVSLAA

SEQ ID NO: 212 - protein, 374 aa, Artificial Sequence
  Fusion Protein STF2delta. E7
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVHGAPVDPASPWTENPLQKIDAALAQVDALRSDLGAVQNRFNSAITNLGNTVNNLSEARSRIEDSDYATEVSNMSRAQILQQAGTSVLAQANQVPQNVLSLLAHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 213 - protein, 434 aa, Artificial Sequence
  Fusion Protein STF2delta. E6
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVHGAPVDPASPWTENPLQKIDAALAQVDALRSDLGAVQNRFNSAITNLGNTVNNLSEARSRIEDSDYATEVSNMSRAQILQQAGTSVLAQANQVPQNVLSLLAHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 214 - protein, 531 aa, Artificial Sequence
  Fusion Protein STF2delta. E6E7
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVHGAPVDPASPWTENPLQKIDAALAQVDALRSDLGAVQNRFNSAITNLGNTVNNLSEARSRIEDSDYATEVSNMSRAQILQQAGTSVLAQANQVPQNVLSLLAHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQLHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 215 - protein, 749 aa, Artificial Sequence
  Fusion Protein STF2delta. L2
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVHGAPVDPASPWTENPLQKIDAALAQVDALRSDLGAVQNRFNSAITNLGNTVNNLSEARSRIEDSDYATEVSNMSRAQILQQAGTSVLAQANQVPQNVLSLLARHKRSAKRTKRASATQLYKTCKQAGTCPPDIIPKVEGKTIADQILQYGSMGVFFGGLGIGTGSGTGGRTGYIPLGTRPPTATDTLAPVRPPLTVDPVGPSDPSIVSLVEETSFIDAGAPTSVPSIPPDVSGFSITTSTDTTPAILDINNTVTTVTTHNNPTFTDPSVLQPPTPAETGGHFTLSSSTISTHNYEEIPMDTFIVSTNPNTVTSSTPIPGSRPVARLGLYSRTTQQVKVVDPAFVTTPTKLITYDNPAYEGIDVDNTLYFSSNDNSINIAPDPDFLDIVALHRPALTSRRTGIRYSRIGNKQTLRTRSGKSIGAKVHYYYDFSTIDPAEEIELQTITPSTYTTTSHAASPTSINNGLYDIYADDFITDTSTTPVPSVPSTSLSGYIPANTTIPFGGAYNIPLVSGPDIPINITDQAPSLIPIVPGSPQYTIIADAGDFYLHPSYYMLRKRRKRLPYFFSDVSLAA

SEQ ID NO: 216 - protein, 104 aa, Artificial Sequence
  E7 protein
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPHHHHHH

SEQ ID NO: 217 - protein, 98 aa, Artificial Sequence; identical to SEQ ID NO: 151
  E7 protein

SEQ ID NO: 218 - protein, 164 aa, Artificial Sequence
  E7 protein
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQLHHHHHH

SEQ ID NO: 219 - protein, 158 aa, Artificial Sequence; identical to SEQ ID NO: 136
  E7 protein

SEQ ID NO: 220 - protein, 261 aa, Artificial Sequence
  E7 protein
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQLHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPHHHHHH

SEQ ID NO: 221 - protein, 255 aa, Artificial Sequence
  E7 protein
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQLHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 222 - protein, 479 aa, Artificial Sequence
  E7 protein
MRHKRSAKRTKRASATQLYKTCKQAGTCPPDIIPKVEGKTIADQILQYGSMGVFFGGLGIGTGSGTGGRTGYIPLGTRPPTATDTLAPVRPPLTVDPVGPSDPSIVSLVEETSFIDAGAPTSVPSIPPDVSGFSITTSTDTTPAILDINNTVTTVTTHNNPTFTDPSVLQPPTPAETGGHFTLSSSTISTHNYEEIPMDTFIVSTNPNTVTSSTPIPGSRPVARLGLYSRTTQQVKVVDPAFVTTPTKLITYDNPAYEGIDVDNTLYFSSNDNSINIAPDPDFLDIVALHRPALTSRRTGIRYSRIGNKQTLRTRSGKSIGAKVHYYYDFSTIDPAEEIELQTITPSTYTTTSHAASPTSINNGLYDIYADDFITDTSTTPVPSVPSTSLSGYIPANTTIPFGGAYNIPLVSGPDIPINITDQAPSLIPIVPGSPQYTIIADAGDFYLHPSYYMLRKRRKRLPYFFSDVSLAAHHHHHH

SEQ ID NO: 223 - protein, 473 aa, Artificial Sequence; identical to SEQ ID NO: 166
  E7 protein

SEQ ID NO: 224 - protein, 5 aa, Artificial Sequence
  CR2 homology region
  VARIANT 2, 4: Xaa = Any Amino Acid
LXCXE

SEQ ID NO: 225 - DNA, 1605 bp, Artificial Sequence
  HPV18 L1 protein
cattaccatctactacctctgtatggcccattgtatcacccacagcccctgcctctacacagtatattggtatacatggtacacattattatttgtggccattatattattttattcctaaaaagcgtaaacgtgttccctatttttttgcagatggctttgtggcggcctagtgacaataccgtataccttccacctccttctgtggcaagagttgtaaatactgatgattatgtgactcgcacaagcatattttatcatgctggcagctctagattattaactgttggtaatccatattttagggttcctgcaagtggtggcaataagcaggatattcctaaggtttctgcataccaatatagagtatttcgggtgcagttacctgacccaaataaatttggtttacctgataatagtatttataatcctgaaacacaacgtttagtgtgggcctgtgctggagtggaaattggccgtggtcagcctttaggtgttggccttagtgggcatccattttataataaattagatgacactgaaagttcccatgccgctacgtctaatgtttctgaggacgttagggacaatgtgtctgtagattataagcagacacagttatgtattttgggctgtgcccctgctattggggaacactgggctaaaggcactgcttgtaaatcgcgtcctttatcacagggcgattgcccccctttagaacttaaaaacacagttttggaagatggtgatatggtagatactggatatggtgccatggactttagtacattgcaagatactaaatgtgaggtaccattggatatttgtcagtctatttgtaaatatcctgattatttacaaatgtctgcagatccttatggggattccatgtttttttgcttacggcgtgagcagctttttgctaggcatttttggaatagggcaggtactatgggtgacactgtgcctcaatccttatatattaaaggcacaggtatgcgtgcttcacctggcagctgtgtgtattctccctctccaagtggctctattgttacctctgactcccagttgtttaataaaccatattggttacataaggcacagggtcataacaatggtatctgctggcataatcaattatttgttactgtggtagataccactcgtagtaccaatttaacaatatgtgcttctacacagtctcctgtacctgggcaatatgatgctaccaaatttaagcagtatagcagacatgttgaagaatatgatttgcagtttatttttcagttatgtactattactttaactgcagatgttatgtcctatattcatagtatgaatagcagtattttagaggattggaactttggtgttccccccccgccaactactagtttggtggatacatatcgttttgtacaatctgttgctattacctgtcaaaaggatgctgcaccagctgaaaataaggatccctatgataggttaaagttttggaatgtggatttaaaggaaaagttttctttggacttagatcaatatccccttggacgtaaatttttggtccaggctggattgcgtcgcaagcccaccataggccctcgt

SEQ ID NO: 226 - protein, 4 aa, Artificial Sequence
  N-glycosylation site
  VARIANT 2: Xaa = Any Amino Acid
NXST

SEQ ID NO: 227 - DNA, 480 bp, Artificial Sequence
  HPV16 E6
atgcatcaaaaacgtactgctatgttccaagatccacaagagcgtccacgtaaactgccgcagctgtgcaccgaactgcagaccactatccacgatatcattctggaatgcgtgtattgcaaacaacagctgctgcgtcgtgaagtctacgattttgcctttcgcgacctgtgcatcgtttatcgcgacggtaacccgtatgctgtctgcgacaaatgcctgaaattttacagcaaaatctccgagtatcgtcattactgctattctctgtacggcacgactctggaacagcagtataacaaaccgctgtgcgacctgctgattcgttgcattaactgccagaagccgctgtgtccggaagaaaaacaacgtcacctggacaaaaagcagcgcttccacaacatccgtggtcgttggaccggccgttgcatgagctgctgccgctccagccgtacccgccgtgaaactcagctgtaatga

SEQ ID NO: 228 - DNA, 498 bp, Artificial Sequence
  HPV16 E6 His6
atgcatcaaaaacgtactgctatgttccaagatccacaagagcgtccacgtaaactgccgcagctgtgcaccgaactgcagaccactatccacgatatcattctggaatgcgtgtattgcaaacaacagctgctgcgtcgtgaagtctacgattttgcctttcgcgacctgtgcatcgtttatcgcgacggtaacccgtatgctgtctgcgacaaatgcctgaaattttacagcaaaatctccgagtatcgtcattactgctattctctgtacggcacgactctggaacagcagtataacaaaccgctgtgcgacctgctgattcgttgcattaactgccagaagccgctgtgtccggaagaaaaacaacgtcacctggacaaaaagcagcgcttccacaacatccgtggtcgttggaccggccgttgcatgagctgctgccgctccagccgtacccgccgtgaaactcagctgcatcaccatcaccatcactaatga

SEQ ID NO: 229 - DNA, 5 bp, Artificial Sequence
  Flagellin
gvtgt

SEQ ID NO: 230 - DNA, 1590 bp, Artificial Sequence
  Fusion Protein SFT2.HPV16 E6 CTL His6
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaaaaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtactgagcctgttggcgatgctgcgtcgcgaggtttatgatttcgcatttcgtgatctgtgcattgtgcaccaccatcatcaccactaa

SEQ ID NO: 231 - protein, 529 aa, Artificial Sequence
  Fusion Protein SFT2.HPV16 E6 CTL His6
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLAMLRREVYDFAFRDLCIVHHHHHH

SEQ ID NO: 232 - DNA, 1736 bp, Artificial Sequence
  Fusion Protein SFT2.HPV16 4XE6 CTL His6
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaaaaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtactgagcctgttggcgatgctgcgtcgcgaggtttatgatttcgcatttcgtgatctgtgcattgtgctgcgtcgcgaagtctacgatttcgcgtttcgtgacttgtgcatcgtcctgcgtcgtgaagtctatgactttgcgtttcgcgatctgtgtattgtcctgcgccgtgaagtgtacgacttcgccttccgtgacctgtgtatcgttcaccaccatcatcaccactaata

SEQ ID NO: 233 - protein, 577 aa, Artificial Sequence
  Fusion Protein SFT2.HPV16 4XE6 CTL His6
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLAMLRREVYDFAFRDLCIVLRREVYDFAFRDLCIVLRREVYDFAFRDLCIVLRREVYDFAFRDLCIVHHHHHH

SEQ ID NO: 234 - DNA, 1820 bp, Artificial Sequence
  Fusion Protein SFT2.HPV16 E7
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaaaaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaactacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattctgcagcaggccggtacttccgttctggcgcaggctaaccaggtcccgcagaacgtgctgagcctgttagcgcatggcgacaccccaactctgcatgaatacatgctggacctgcagccggaaacgaccgatctgtattgttacgaacagctgaacgattcctccgaagaagaagacgagatcgacggtccggcaggtcaagcggaaccagatcgtgcgcactataatatcgtgactttctgttgtaaatgtgactccaccctgcgtctgtgtgtgcagtctacccacgttgatatccgcaccctggaagatctgctgatgggcaccctgggtatcgtgtgcccgatctgcagccagaaaccgtaataagctga

SEQ ID NO: 235 - DNA, 1133 bp, Artificial Sequence
  Fusion Protein SFT2delta.HPV16 E7
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcatggagcgccggtggatcctgctagcccatggaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttagcgcatggcgacaccccaactctgcatgaatacatgctggacctgcagccggaaacgaccgatctgtattgttacgaacagctgaacgattcctccgaagaagaagacgagatcgacggtccggcaggtcaagcggaaccagatcgtgcgcactataatatcgtgactttctgttgtaaatgtgactccaccctgcgtctgtgtgtgcagtctacccacgttgatatccgcaccctggaagatctgctgatgggcaccctgggtatcgtgtgcccgatctgcagccagaaaccgtaataagctga

SEQ ID NO: 236 - DNA, 305 bp, Artificial Sequence
  HPV16 E7
atgcatggcgacaccccaactctgcatgaatacatgctggacctgcagccggaaacgaccgatctgtattgttacgaacagctgaacgattcctccgaagaagaagacgagatcgacggtccggcaggtcaagcggaaccagatcgtgcgcactataatatcgtgactttctgttgtaaatgtgactccaccctgcgtctgtgtgtgcagtctacccacgttgatatccgcaccctggaagatctgctgatgggcaccctgggtatcgtgtgcccgatctgcagccagaaaccgtaataagctga

SEQ ID NO: 237 - DNA, 323 bp, Artificial Sequence
  HPV16 E7 His6
atgcatggcgacaccccaactctgcatgaatacatgctggacctgcagccggaaacgaccgatctgtattgttacgaacagctgaacgattcctccgaagaagaagacgagatcgacggtccggcaggtcaagcggaaccagatcgtgcgcactataatatcgtgactttctgttgtaaatgtgactccaccctgcgtctgtgtgtgcagtctacccacgttgatatccgcaccctggaagatctgctgatgggcaccctgggtatcgtgtgcccgatctgcagccagaaaccgcatcaccatcaccatcactaataagctga

SEQ ID NO: 238 - DNA, 1602 bp, Artificial Sequence
  Fusion Protein STF2.HPV16 E7 CTL His6
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaaaaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtactgagcctgttggcgatgcaagccgagccggatcgtgctcactacaatatcgttacgttttgttgcaaatgtgaccaccatcatcaccaccactaataa

SEQ ID NO: 239 - protein, 532 aa, Artificial Sequence
  Fusion Protein STF2.HPV16 E7 CTL His6
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLAMQAEPDRAHYNIVTFCCKCDHHHHHH

SEQ ID NO: 240 - DNA, 1773 bp, Artificial Sequence
  Fusion Protein STF2.HPV16 4XE7 CTL His6
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaaaaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtactgagcctgttggcgatgcaagccgagccggatcgtgctcactacaatatcgttacgttttgttgcaaatgtgaccaggcggaaccggaccgcgcacactataacattgttaccttctgctgcaagtgtgatcaggcggaaccggaccgtgcgcattacaatatcgtcaccttctgctgtaagtgcgatcaagcagagccagatcgtgcgcattacaacattgtgaccttttgctgtaaatgcgaccaccatcatcaccaccactaataa

SEQ ID NO: 241 - protein, 589 aa, Artificial Sequence
  Fusion Protein STF2.HPV16 4XE7 CTL His6
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLAMQAEPDRAHYNIVTFCCKCDQAEPDRAHYNIVTFCCKCDQAEPDRAHYNIVTFCCKCDQAEPDRAHYNIVTFCCKCDHHHHHH

SEQ ID NO: 242 - DNA, 2293 bp, Artificial Sequence
  Fusion Protein STF2.HPV16 E6 E7
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaaaaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaactacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattctgcagcaggccggtacttccgttctggcgcaggctaaccaggtcccgcagaacgtgctgagcctgttagcgcatcaaaaacgcactgcaatgttccaggatccgcaagaacgtccacgtaagctgccgcagctgtgcactgagctgcaaactactatccatgacatcatcctggagtgtgtgtattgcaaacaacagctgctgcgtcgtgaagtgtacgatttcgccttccgtgacctgtgcatcgtttatcgtgacggtaatccatacgccgtttgcgacaaatgcctgaaattttacagcaaaatctccgaataccgtcactattgctactctctgtacggcacgaccctggaacagcaatataacaaaccgctgtgcgacctgctgattcgttgtattaactgccagaagcctctgtgtccggaggaaaaacagcgtcacctggacaagaaacagcgctttcataacatccgcggtcgctggactggtcgttgtatgtcctgctgccgttcttcccgcactcgccgcgagactcagctgcatggtgatactcctactctgcatgagtacatgctggacctgcaaccggaaaccactgatctgtattgttacgaacagctgaacgatagctctgaagaagaggacgaaatcgacggtccagcaggtcaggctgaaccggaccgcgctcattataacatcgttacgttttgctgcaaatgcgacagcaccctgcgtctgtgtgttcagtctacccacgtagatattcgtactctggaggatctgctgatgggcactctgggcatcgtttgcccgatctgttcccagaaaccgtaataagctgagc

SEQ ID NO: 243 - DNA, 1606 bp, Artificial Sequence
  Fusion Protein STF2delta.HPV16 E6 E7
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcatggagcgccggtggatcctgctagcccatggaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttagcgcatcaaaaacgcactgcaatgttccaggatccgcaagaacgtccacgtaagctgccgcagctgtgcactgagctgcaaactactatccatgacatcatcctggagtgtgtgtattgcaaacaacagctgctgcgtcgtgaagtgtacgatttcgccttccgtgacctgtgcatcgtttatcgtgacggtaatccatacgccgtttgcgacaaatgcctgaaattttacagcaaaatctccgaataccgtcactattgctactctctgtacggcacgaccctggaacagcaatataacaaaccgctgtgcgacctgctgattcgttgtattaactgccagaagcctctgtgtccggaggaaaaacagcgtcacctggacaagaaacagcgctttcataacatccgcggtcgctggactggtcgttgtatgtcctgctgccgttcttcccgcactcgccgcgagactcagctgcatggtgatactcctactctgcatgagtacatgctggacctgcaaccggaaaccactgatctgtattgttacgaacagctgaacgatagctctgaagaagaggacgaaatcgacggtccagcaggtcaggctgaaccggaccgcgctcattataacatcgttacgttttgctgcaaatgcgacagcaccctgcgtctgtgtgttcagtctacccacgtagatattcgtactctggaggatctgctgatgggcactctgggcatcgtttgcccgatctgttcccagaaaccgtaataagctgagc

SEQ ID NO: 244 - DNA, 778 bp, Artificial Sequence
  HPV16 E6E7
atgcatcaaaaacgcactgcaatgttccaggatccgcaagaacgtccacgtaagctgccgcagctgtgcactgagctgcaaactactatccatgacatcatcctggagtgtgtgtattgcaaacaacagctgctgcgtcgtgaagtgtacgatttcgccttccgtgacctgtgcatcgtttatcgtgacggtaatccatacgccgtttgcgacaaatgcctgaaattttacagcaaaatctccgaataccgtcactattgctactctctgtacggcacgaccctggaacagcaatataacaaaccgctgtgcgacctgctgattcgttgtattaactgccagaagcctctgtgtccggaggaaaaacagcgtcacctggacaagaaacagcgctttcataacatccgcggtcgctggactggtcgttgtatgtcctgctgccgttcttcccgcactcgccgcgagactcagctgcatggtgatactcctactctgcatgagtacatgctggacctgcaaccggaaaccactgatctgtattgttacgaacagctgaacgatagctctgaagaagaggacgaaatcgacggtccagcaggtcaggctgaaccggaccgcgctcattataacatcgttacgttttgctgcaaatgcgacagcaccctgcgtctgtgtgttcagtctacccacgtagatattcgtactctggaggatctgctgatgggcactctgggcatcgtttgcccgatctgttcccagaaaccgtaataagctgagc

SEQ ID NO: 245 - DNA, 796 bp, Artificial Sequence
  HPV16 E6E7 His6
atgcatcaaaaacgcactgcaatgttccaggatccgcaagaacgtccacgtaagctgccgcagctgtgcactgagctgcaaactactatccatgacatcatcctggagtgtgtgtattgcaaacaacagctgctgcgtcgtgaagtgtacgatttcgccttccgtgacctgtgcatcgtttatcgtgacggtaatccatacgccgtttgcgacaaatgcctgaaattttacagcaaaatctccgaataccgtcactattgctactctctgtacggcacgaccctggaacagcaatataacaaaccgctgtgcgacctgctgattcgttgtattaactgccagaagcctctgtgtccggaggaaaaacagcgtcacctggacaagaaacagcgctttcataacatccgcggtcgctggactggtcgttgtatgtcctgctgccgttcttcccgcactcgccgcgagactcagctgcatggtgatactcctactctgcatgagtacatgctggacctgcaaccggaaaccactgatctgtattgttacgaacagctgaacgatagctctgaagaagaggacgaaatcgacggtccagcaggtcaggctgaaccggaccgcgctcattataacatcgttacgttttgctgcaaatgcgacagcaccctgcgtctgtgtgttcagtctacccacgtagatattcgtactctggaggatctgctgatgggcactctgggcatcgtttgcccgatctgttcccagaaaccgcatcaccatcaccatcactaataagctgagc

SEQ ID NO: 246 - DNA, 2947 bp, Artificial Sequence
  STF2. HPV16 L2
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaaaaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaactacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattctgcagcaggccggtacttccgttctggcgcaggctaaccaggtcccgcagaacgtgctgagcctgttagcgcgtcataaacgttctgcaaagcgtactaagcgtgctagcgcaactcagctgtacaaaacgtgtaaacaggcgggtacgtgtccgcctgacatcatcccgaaagttgaaggcaaaactatcgcggaccagattctgcagtacggttctatgggtgttttttttggtggtctgggtatcggcaccggttctggcaccggtggccgcacgggttatattccgctgggtactcgtccgccgactgcgaccgataccctggcaccggtacgtccgccgctgaccgtcgacccggttggtccgtctgatccgtctattgtgtctctggtcgaagagactagcttcattgacgctggtgcgccgacttctgttccgagcatcccgcctgatgttagcggtttctccatcactacttccaccgacaccaccccagctattctggatatcaacaacaccgttaccaccgtgaccacccataacaacccgaccttcaccgacccgtctgtcctgcagccgcctaccccggctgaaaccggcggtcacttcactctgtcttcttccaccatctctacccacaactatgaagagatccctatggacacttttatcgtgtccaccaaccctaacaccgtcacctcttctaccccgattccgggctctcgtccagtggctcgcctgggtctgtactcccgcaccactcagcaggtgaaggttgtcgatccggcgtttgtgaccactccgactaaactgatcacttacgataatccggcttacgaaggtattgatgtagacaatactctgtacttctcttccaatgacaacagcattaacatcgcgccggacccggactttctggatatcgttgcactgcaccgcccagccctgacctcccgtcgtactggtatccgttacagccgtatcggcaacaagcagaccctgcgtacccgctctggtaaatccatcggcgccaaagtacactactattacgacttttccactatcgatccggcagaagagattgaactgcagaccatcactccgtccacttatactaccacctcccacgctgcatccccgactagcattaacaatggtctgtacgatatctatgccgatgatttcattaccgacacgtctaccaccccggttccgtctgtcccgtccacctctctgtccggctatatcccggcgaacactacgattccgttcggtggtgcatacaacattccgctggtgagcggcccggacattccgatcaacatcaccgatcaggctccatctctgatcccgatcgtaccgggttccccacagtacactattattgctgatgcgggtgatttctatctgcatccttcttactacatgctgcgtaaacgtcgtaaacgtctgccatacttcttctctgacgttagcctggctgcttaataagctgagc

SEQ ID NO: 247 - DNA, 2260 bp, Artificial Sequence
  Fusion Protein STF2delta. HPV16 L2
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcatggagcgccggtggatcctgctagcccatggaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttagcgcgtcataaacgttctgcaaagcgtactaagcgtgctagcgcaactcagctgtacaaaacgtgtaaacaggcgggtacgtgtccgcctgacatcatcccgaaagttgaaggcaaaactatcgcggaccagattctgcagtacggttctatgggtgttttttttggtggtctgggtatcggcaccggttctggcaccggtggccgcacgggttatattccgctgggtactcgtccgccgactgcgaccgataccctggcaccggtacgtccgccgctgaccgtcgacccggttggtccgtctgatccgtctattgtgtctctggtcgaagagactagcttcattgacgctggtgcgccgacttctgttccgagcatcccgcctgatgttagcggtttctccatcactacttccaccgacaccaccccagctattctggatatcaacaacaccgttaccaccgtgaccacccataacaacccgaccttcaccgacccgtctgtcctgcagccgcctaccccggctgaaaccggcggtcacttcactctgtcttcttccaccatctctacccacaactatgaagagatccctatggacacttttatcgtgtccaccaaccctaacaccgtcacctcttctaccccgattccgggctctcgtccagtggctcgcctgggtctgtactcccgcaccactcagcaggtgaaggttgtcgatccggcgtttgtgaccactccgactaaactgatcacttacgataatccggcttacgaaggtattgatgtagacaatactctgtacttctcttccaatgacaacagcattaacatcgcgccggacccggactttctggatatcgttgcactgcaccgcccagccctgacctcccgtcgtactggtatccgttacagccgtatcggcaacaagcagaccctgcgtacccgctctggtaaatccatcggcgccaaagtacactactattacgacttttccactatcgatccggcagaagagattgaactgcagaccatcactccgtccacttatactaccacctcccacgctgcatccccgactagcattaacaatggtctgtacgatatctatgccgatgatttcattaccgacacgtctaccaccccggttccgtctgtcccgtccacctctctgtccggctatatcccggcgaacactacgattccgttcggtggtgcatacaacattccgctggtgagcggcccggacattccgatcaacatcaccgatcaggctccatctctgatcccgatcgtaccgggttccccacagtacactattattgctgatgcgggtgatttctatctgcatccttcttactacatgctgcgtaaacgtcgtaaacgtctgccatacttcttctctgacgttagcctggctgcttaataagctgagc

SEQ ID NO: 248 - DNA, 1432 bp, Artificial Sequence
  HPV16 L2
atgcgtcataaacgttctgcaaagcgtactaagcgtgctagcgcaactcagctgtacaaaacgtgtaaacaggcgggtacgtgtccgcctgacatcatcccgaaagttgaaggcaaaactatcgcggaccagattctgcagtacggttctatgggtgttttttttggtggtctgggtatcggcaccggttctggcaccggtggccgcacgggttatattccgctgggtactcgtccgccgactgcgaccgataccctggcaccggtacgtccgccgctgaccgtcgacccggttggtccgtctgatccgtctattgtgtctctggtcgaagagactagcttcattgacgctggtgcgccgacttctgttccgagcatcccgcctgatgttagcggtttctccatcactacttccaccgacaccaccccagctattctggatatcaacaacaccgttaccaccgtgaccacccataacaacccgaccttcaccgacccgtctgtcctgcagccgcctaccccggctgaaaccggcggtcacttcactctgtcttcttccaccatctctacccacaactatgaagagatccctatggacacttttatcgtgtccaccaaccctaacaccgtcacctcttctaccccgattccgggctctcgtccagtggctcgcctgggtctgtactcccgcaccactcagcaggtgaaggttgtcgatccggcgtttgtgaccactccgactaaactgatcacttacgataatccggcttacgaaggtattgatgtagacaatactctgtacttctcttccaatgacaacagcattaacatcgcgccggacccggactttctggatatcgttgcactgcaccgcccagccctgacctcccgtcgtactggtatccgttacagccgtatcggcaacaagcagaccctgcgtacccgctctggtaaatccatcggcgccaaagtacactactattacgacttttccactatcgatccggcagaagagattgaactgcagaccatcactccgtccacttatactaccacctcccacgctgcatccccgactagcattaacaatggtctgtacgatatctatgccgatgatttcattaccgacacgtctaccaccccggttccgtctgtcccgtccacctctctgtccggctatatcccggcgaacactacgattccgttcggtggtgcatacaacattccgctggtgagcggcccggacattccgatcaacatcaccgatcaggctccatctctgatcccgatcgtaccgggttccccacagtacactattattgctgatgcgggtgatttctatctgcatccttcttactacatgctgcgtaaacgtcgtaaacgtctgccatacttcttctctgacgttagcctggctgcttaataagctgagc

SEQ ID NO: 249 - DNA, 1450 bp, Artificial Sequence
  HPV16 L2 His6
atgcgtcataaacgttctgcaaagcgtactaagcgtgctagcgcaactcagctgtacaaaacgtgtaaacaggcgggtacgtgtccgcctgacatcatcccgaaagttgaaggcaaaactatcgcggaccagattctgcagtacggttctatgggtgttttttttggtggtctgggtatcggcaccggttctggcaccggtggccgcacgggttatattccgctgggtactcgtccgccgactgcgaccgataccctggcaccggtacgtccgccgctgaccgtcgacccggttggtccgtctgatccgtctattgtgtctctggtcgaagagactagcttcattgacgctggtgcgccgacttctgttccgagcatcccgcctgatgttagcggtttctccatcactacttccaccgacaccaccccagctattctggatatcaacaacaccgttaccaccgtgaccacccataacaacccgaccttcaccgacccgtctgtcctgcagccgcctaccccggctgaaaccggcggtcacttcactctgtcttcttccaccatctctacccacaactatgaagagatccctatggacacttttatcgtgtccaccaaccctaacaccgtcacctcttctaccccgattccgggctctcgtccagtggctcgcctgggtctgtactcccgcaccactcagcaggtgaaggttgtcgatccggcgtttgtgaccactccgactaaactgatcacttacgataatccggcttacgaaggtattgatgtagacaatactctgtacttctcttccaatgacaacagcattaacatcgcgccggacccggactttctggatatcgttgcactgcaccgcccagccctgacctcccgtcgtactggtatccgttacagccgtatcggcaacaagcagaccctgcgtacccgctctggtaaatccatcggcgccaaagtacactactattacgacttttccactatcgatccggcagaagagattgaactgcagaccatcactccgtccacttatactaccacctcccacgctgcatccccgactagcattaacaatggtctgtacgatatctatgccgatgatttcattaccgacacgtctaccaccccggttccgtctgtcccgtccacctctctgtccggctatatcccggcgaacactacgattccgttcggtggtgcatacaacattccgctggtgagcggcccggacattccgatcaacatcaccgatcaggctccatctctgatcccgatcgtaccgggttccccacagtacactattattgctgatgcgggtgatttctatctgcatccttcttactacatgctgcgtaaacgtcgtaaacgtctgccatacttcttctctgacgttagcctggctgctcatcaccatcaccatcactaataagctgagc

SEQ ID NO: 250 - protein, 10 aa, Artificial Sequence
  HPVE6 peptide
EVYDFAFRDL

(19) United States
(12) Patent Application Publication
(10) Pub. No.: US 2010/0303847 A1
(43) Pub. Date: Dec. 2, 2010

(54) COMPOSITIONS OF TOLL-LIKE RECEPTOR AGONISTS AND PAPILLOMAVIRUS ANTIGENS AND METHODS OF USE THEREOF

(76) Inventors: Valerian Nakaar, Hamden, CT (US); James W. Huleatt, Nazareth, PA (US); Thomas J. Powell, Madison, CT (US); Albert E. Price, New Haven, CT (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/787,649

(22) Filed: May 26, 2010

Related U.S. Application Data
(63) Continuation of application No. PCT/US2008/013149, filed on Nov. 26, 2008.
(60) Provisional application No. 61/004,680, filed on Nov. 29, 2007.

(51) Int. Cl. A61K 39/12; A61P 37/04

(52) U.S. Cl. 424/186.1

(57) ABSTRACT

Compositions that include at least one protein that activates a Toll-like Receptor and includes at least a portion of at least one Toll-like Receptor agonist and at least a portion of at least one papillomavirus suppressor binding protein, papillomavirus transforming protein and papillomavirus capsid protein can be employed in methods that stimulate an immune response in a subject, in particular, a protective immune response in a subject. Compositions can further include an adjuvant and a carrier protein.

### RELATED APPLICATION(S)

This application is a continuation of International Application No. PCT/US2008/013149, which designated the United States and was filed on Nov. 26, 2008, published in English, which claims the benefit of U.S. Provisional Application No. 61/004,680, filed on Nov. 29, 2007. The entire teachings of the above applications are incorporated herein by reference.

### BACKGROUND OF THE INVENTION

Papillomaviruses are DNA-viruses that infect the skin and mucous membranes of humans and a variety of animals. Over 100 different human papillomavirus (HPV) types have been identified. HPVs are transmitted by skin-to-skin contact. HPV infection can result in the abnormal growth and proliferation of infected cells. Gardasil® is a vaccine to prevent infection with some types of HPV (6, 11, 16, and 18), but is generally effective only if administered before an individual is infected with HPV. Thus, there is a need to develop new, improved and effective methods of treatment for preventing and managing disease associated with HPV infection.

### SUMMARY OF THE INVENTION

The present invention relates to compositions that include HPV proteins, such as compositions that stimulate a protective immune response, and methods of making proteins that stimulate a protective immune response in a subject.

In an embodiment, the invention is a composition that includes at least one protein that includes at least a portion of at least one Toll-like Receptor agonist and at least a portion of at least one papillomavirus tumor suppressor binding protein, wherein the protein activates a Toll-like Receptor.

In another embodiment, the invention is a composition that includes at least one protein that includes at least a portion of at least one Toll-like Receptor agonist and at least a portion of at least one papillomavirus transforming protein, wherein the protein activates a Toll-like Receptor agonist.

In yet another embodiment, the invention is a composition that includes at least one protein that includes at least a portion of at least one Toll-like Receptor agonist and at least a portion of at least one papillomavirus capsid protein, wherein the protein activates a Toll-like Receptor.

In still another embodiment, the invention is a composition that includes at least a portion of at least one Toll-like Receptor 5 agonist and at least a portion of at least one papillomavirus protein, wherein the papillomavirus protein includes at least one member selected from the group consisting of a papillomavirus E6 protein, a papillomavirus E7 protein and a papillomavirus L2 protein.

An additional embodiment of the invention is a method of stimulating an immune response in a subject, comprising the step of administering to the subject a composition that includes at least one protein that includes at least a portion of at least one Toll-like Receptor agonist and at least a portion of at least one papillomavirus tumor suppressor binding protein, wherein the protein activates a Toll-like Receptor.

Another embodiment of the invention is a method of stimulating an immune response in a subject, comprising the step of administering to the subject a composition that includes at least a portion of at least one Toll-like Receptor agonist and at least a portion of at least one papillomavirus transforming protein, wherein the protein activates a Toll-like Receptor agonist.

A further embodiment of the invention is a method of stimulating an immune response in a subject, comprising the step of administering to the subject a composition that includes at least a portion of at least one Toll-like Receptor agonist and at least a portion of at least one papillomavirus capsid protein, wherein the protein activates a Toll-like Receptor.

In still another embodiment, the invention is a method of stimulating an immune response in a subject, comprising the step of administering to the subject a composition that includes a papillomavirus protein, wherein the papillomavirus protein includes at least a portion of at least a portion of a Toll-like Receptor 5 agonist and at least a portion of at least one member selected from the group consisting of a papillomavirus E6 protein, a papillomavirus E7 protein and a papillomavirus L2 protein.

The methods and composition of the invention can be employed to stimulate an immune response, in particular, a protective immune response, in a subject. Advantages of the claimed invention include, for example, cost effective methods and compositions that can be produced in relatively large quantities for use in the prevention and treatment of disease associated with papillomavirus infection. The claimed compositions and methods can be employed to prevent or treat papillomavirus infection, in particular human papillomavirus (HPV) and, therefore, avoid serious illness and death consequent to HPV infection.

### BRIEF DESCRIPTION OF THE FIGURES

FIG. 1 depicts flagellin—HPV constructs.

FIG. 2 depicts constructs of flagellin and HPV antigen fusion proteins. The constructs include full length Salmonella typhimurium fljb (STF2) and STF2Δ, a flagellin lacking a portion of the hinge region.

FIG. 3 depicts the activation of an antigen-presenting cell (APC) by Toll-like Receptor (TLR) signaling.

FIG. 4 depicts the D1 domain, D2 domain, TLR5 activation domain and hypervariable (D3 domain) of flagellin.

FIG. 5 depicts the D1 domain, D2 domain, TLR5 activation domain and hypervariable (D3 domain) of flagellin (Yonekura, et al. Nature 424: 643-650 (2003)).

FIG. 6 depicts the HPV16 genome.

FIG. 7 depicts the HPV genome, mRNA and proteins.

FIG. 8 depicts a tripalmitoylated peptide.

FIG. 9 depicts the amino acid sequence of fusion proteins of the invention (STF2.E7; SEQ ID NO: 208 and STF2.E6; SEQ ID NO: 209).

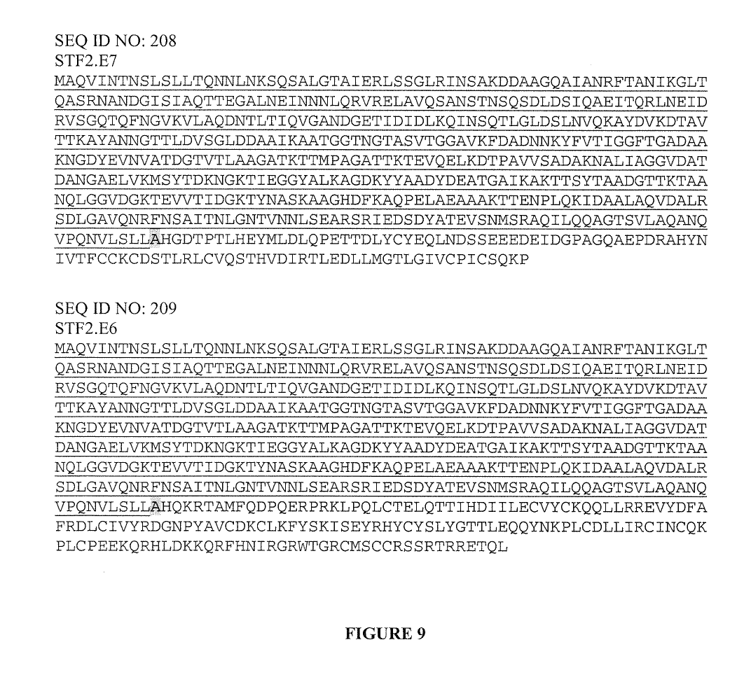

FIG. 10 depicts the amino acid sequence of a fusion protein that includes STF2 and at least a portion of an E6 protein and an E7 protein (STF2.E6E7; SEQ ID NO: 210).

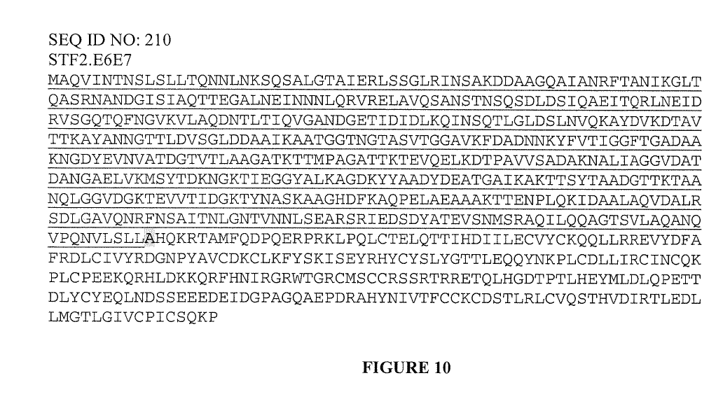

FIG. 11 depicts the amino acid sequence of a fusion protein that includes STF2 and at least a portion of a L2 protein (STF2.L2; SEQ ID NO: 211).

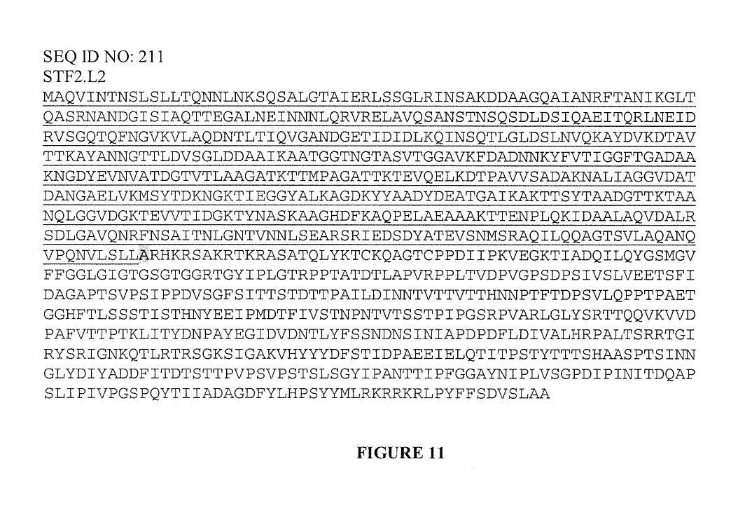

FIG. 12 depicts the amino sequence of a fusion protein that includes STF2Δ and at least a portion of an E7 protein (STF2Δ.E7; SEQ ID NO: 212), STF2Δ and at least a portion of an E6 protein (STF2ΔE6; SEQ ID NO: 213) and STF2Δ and at least a portion of an E6 protein and at least a portion of an E7 protein (STF2Δ.E6E7; SEQ ID NO: 214).

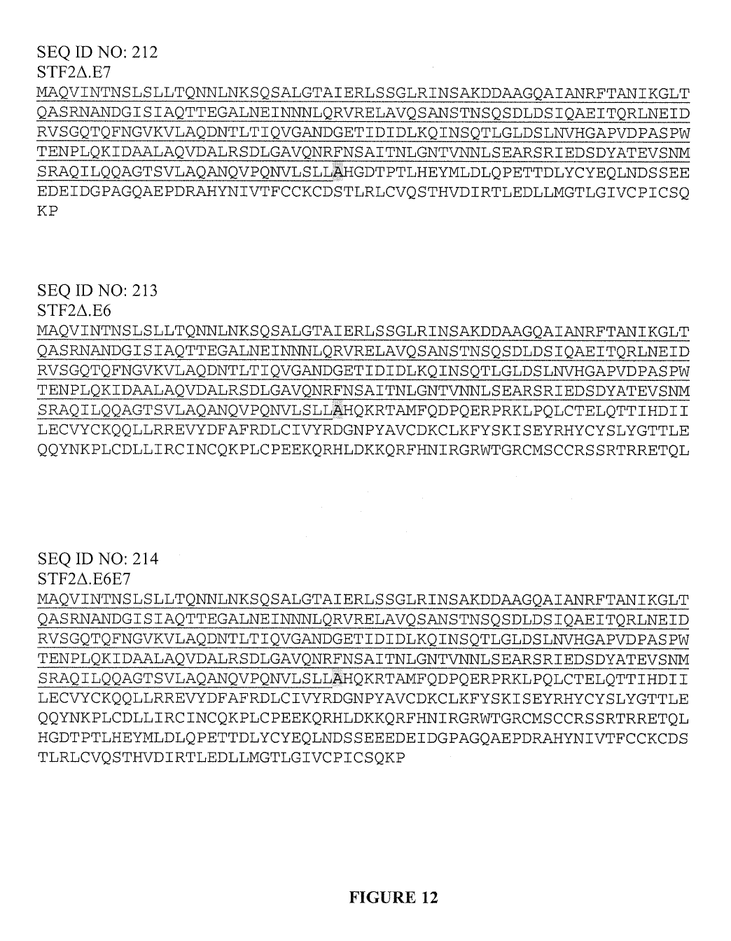

FIG. 13 depicts the amino acid sequence of a fusion protein of STF2Δ and at least a portion of an L2 protein (STF2Δ.L2; SEQ ID NO: 215).

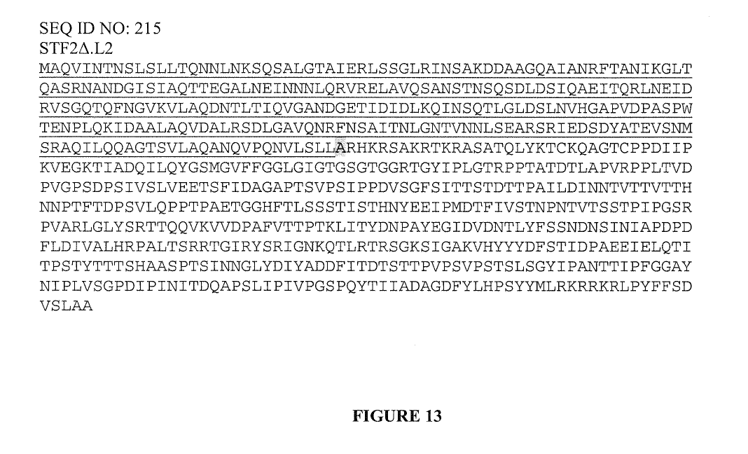

FIG. 14 depicts the amino acid sequence of E7 (SEQ ID NOs: 216 and 217) and E6 proteins (SEQ ID NO: 218 and 219) for use in the compositions of the invention.

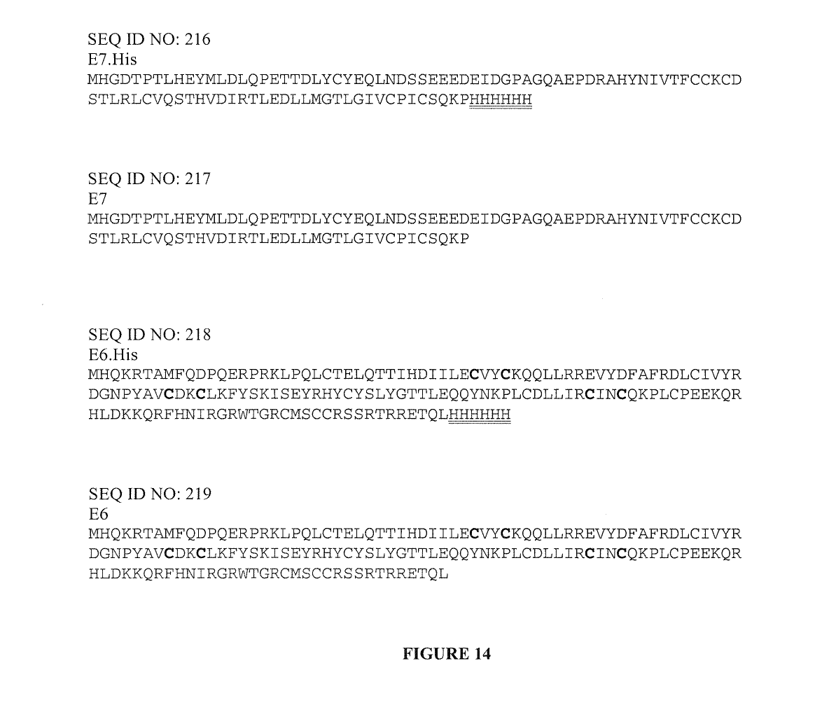

FIG. 15 depicts the amino acid sequences of proteins that include E6 and E7 HPV proteins (E6E7; SEQ ID NOs: 220 and 221) and L2 proteins (SEQ ID NOs: 222 and 223) for use in the compositions of the invention.

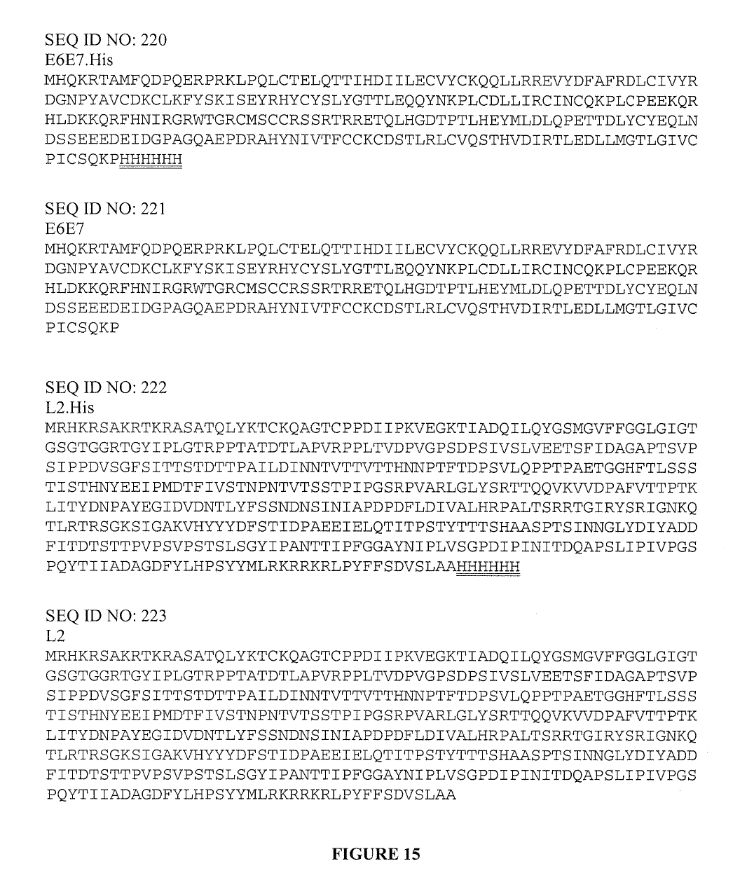

FIG. 16 depicts the amino acid sequence (SEQ ID NO: 98) of a flagellin for use in the compositions of the invention. The hinge region of the flagellin is underlined.

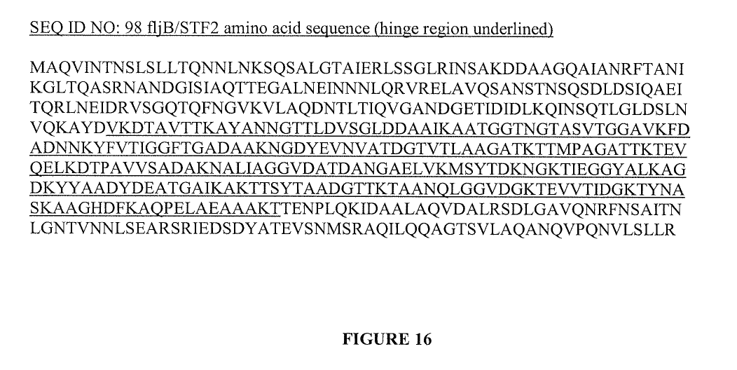

FIG. 17 depicts the nucleic acid sequence encoding a flagellin for use in the compositions of the invention (SEQ ID NO: 108). The nucleic acid sequence encoding the hinge region is underlined.

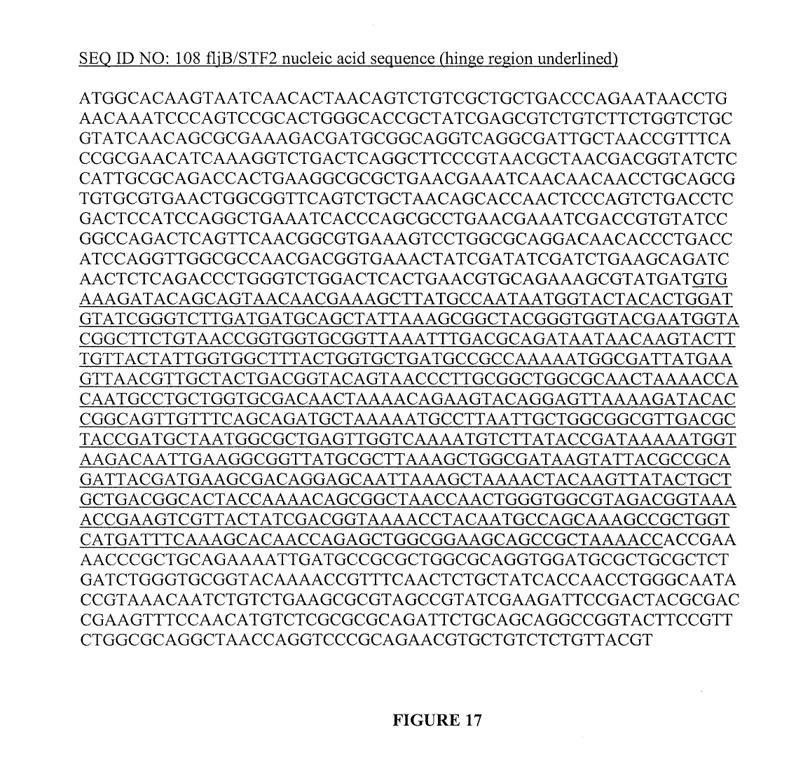

FIG. 18 depicts the amino acid sequence of a flagellin lacking a hinge region (SEQ ID NO: 101) for use in compositions of the invention and the corresponding nucleic acid sequence (SEQ ID NO: 109).

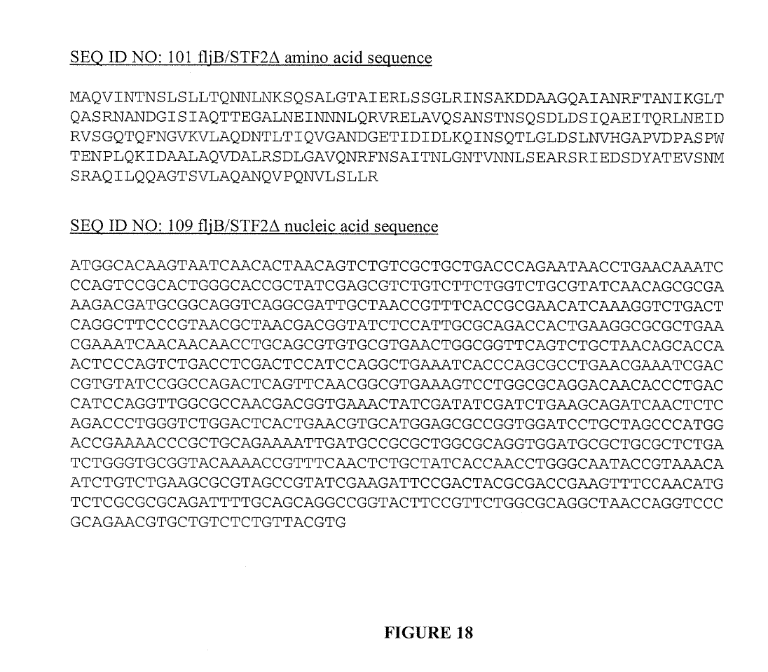

FIG. 19 depicts the amino acid sequence of a flagellin (SEQ ID NO: 106) for use in the compositions of the invention. The hinge region of the flagellin is underlined.

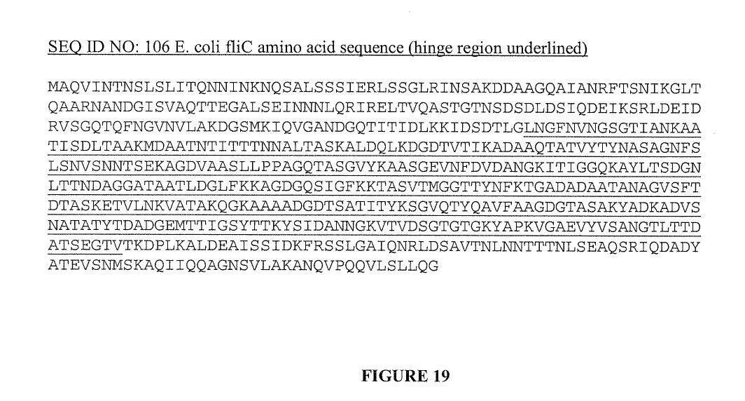

FIG. 20 depicts a nucleic acid sequence (SEQ ID NO: 111) encoding a flagellin for use in compositions of the invention. The nucleic acid sequence encoding the hinge region of the flagellin is underlined.

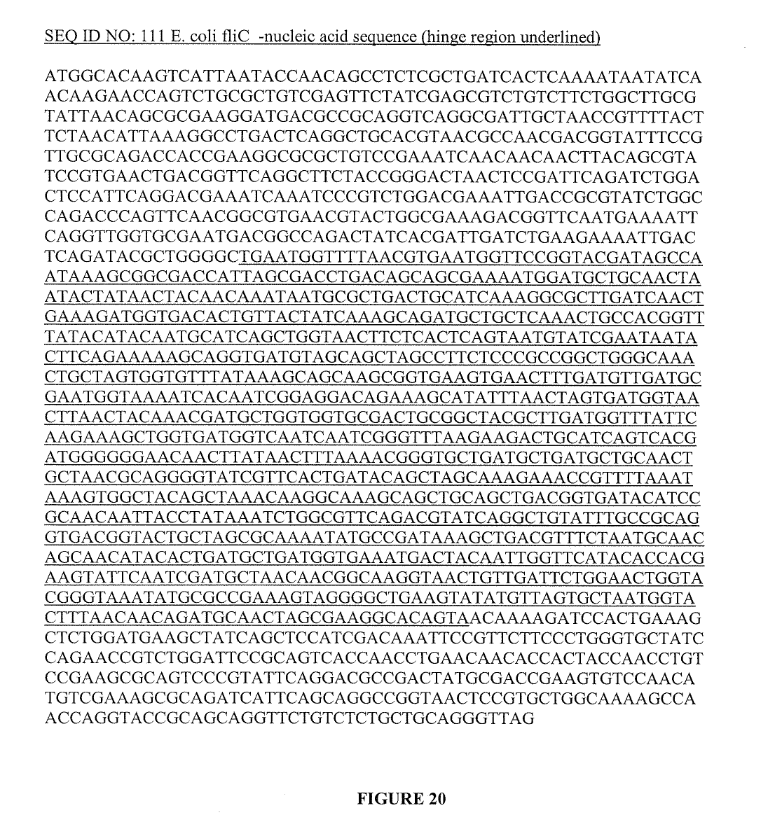

FIG. 21 depicts the amino acid sequence (SEQ ID NO: 102) of flagellin for use in the compositions of the invention. The hinge region of the flagellin is underlined.

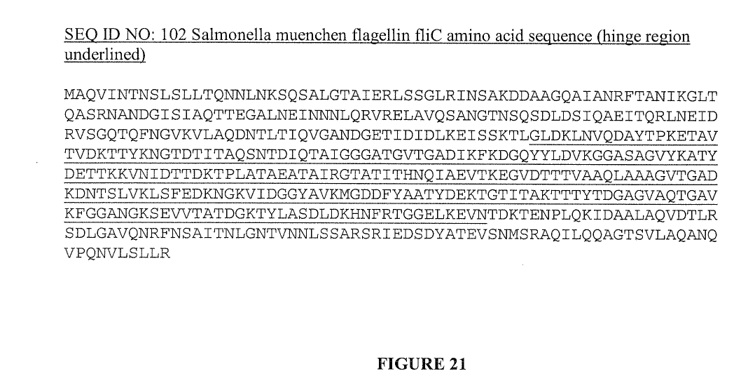

FIG. 22 depicts a nucleic acid sequence (SEQ ID NO: 110) encoding a flagellin for use in the compositions of the invention. The nucleic acid sequence encoding the hinge region of flagellin is underlined.

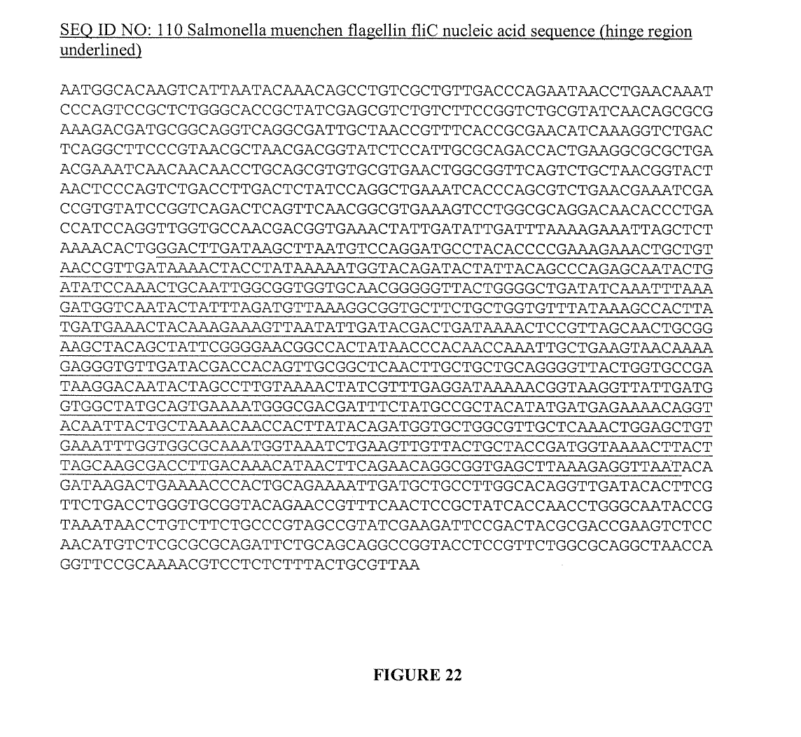

FIGS. 23A and 23B depict HPV16 E6 antigen-specific T-cell responses in response to immunization with STF.2HPV16 E6 (SEQ ID NO: 209) by ELISPOT assay in IFN-γ-positive cells (FIG. 23A; 30 μg or 3 μg STF2.E6 in TITERMAX®) and IL-5-positive cells (FIG. 23B; 30 μg or 3 μg STF2.E6 in TITERMAX®).

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principle features of this invention can be employed in various embodiments without departing from the scope of the invention.

The invention is generally directed to compositions of papillomavirus proteins, in particular, human papillomavirus (HPV) proteins and methods of stimulating an immune response, such as a protective immune response, in a subject employing the compositions described herein.

In an embodiment, the invention is a composition that includes at least one protein that includes at least a portion of at least one Toll-like Receptor agonist and at least a portion of at least one papillomavirus tumor suppressor binding protein, wherein the protein activates a Toll-like Receptor.

“Component,” as used herein in reference to the compositions described herein, refers to constituents of the protein. For example, a “papillomavirus component,” as used herein, refers to part of the protein that includes at least a portion or the entirety of a papillomavirus protein, in particular, a papillomavirus protein that binds to a tumor suppressor protein (e.g., an E6 protein that binds p53), a papillomavirus transforming protein (e.g., an E7 papillomavirus protein that binds a pRB protein; and/or a p107 protein) and a papillomavirus capsid protein (e.g., a major capsid protein, such as an L1 protein, a minor capsid protein, such as an L2 protein). Likewise, a “Toll-like Receptor agonist component,” as used herein, refers to at least part of the protein that includes at least a portion of a Toll-like Receptor agonist. The Toll-like Receptor agonist component can be a flagellin component. “Flagellin component,” as used herein, refers to at least part of the protein that includes at least a portion of or the entirety a flagellin.

“At least a portion,” as used herein in reference to components of the proteins of the invention, means any part or the entirety of the component. For example, at least a portion of a papillomavirus protein can include at least one member selected from the group consisting of an E6, an E7 and an L2 protein. “At least a portion” is also referred to as “fragment.”

Pathogen-associated molecular patterns (PAMPs), such as a flagellin or a bacterial lipoprotein, refer to a class of molecules (e.g., protein, peptide, carbohydrate, lipid, lipopeptide, nucleic acid) found in microorganisms that, when bound to a pattern recognition receptor (PRR), can trigger an innate immune response. The PRR can be a Toll-like Receptor (TLR).

TLRs are the best characterized type of Pattern Recognition Receptor (PRR) expressed on antigen-presenting cells (APC). APC utilize TLRs to survey the microenvironment and detect signals of pathogenic infection by engaging the cognate ligands of TLRs, PAMPs. TLR activation triggers the innate immune response, the first line of defense against pathogenic insult, manifested as release of cytokines, chemokines and other inflammatory mediators; recruitment of phagocytic cells; and important cellular mechanisms which lead to the expression of costimulatory molecules and efficient processing and presentation of antigens to T-cells. TLRs can control both innate and the adaptive immune responses.

Toll-like Receptors were named based on homology to the Drosophila melangogaster Toll protein. Toll-like Receptors are type I transmembrane signaling receptor proteins characterized by an extracellular leucine-rich repeat domain and an intracellular domain homologous to an interleukin 1 receptor. Toll-like Receptors include TLR1, TLR2, TLR3, TLR4, TLR5, TLR6, TLR7, TLR 8, TLR9, TLR10, TLR11 and TLR12.

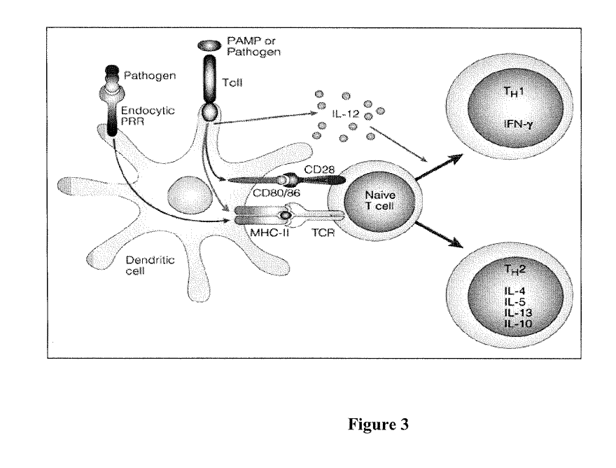

The binding of PAMPs to TLRs activates innate immune pathways. Target cells can result in the display of co-stimulatory molecules on the cell surface, as well as antigenic peptide in the context of major histocompatibility complex molecules (see FIG. 3). The compositions and proteins of the invention include a TLR (e.g., TLR5), promoting differentiation and maturation of the APC, including production and display of co-stimulatory signals (see FIG. 3). The proteins of the invention can be internalized by interaction with TLR and processed through the lysosomal pathway to generate antigenic peptides, which are displayed on the surface in the context of the major histocompatibility complex.

The compositions and proteins of the invention employ TLR agonists that trigger cellular events resulting in the expression of costimulatory molecules, secretion of critical cytokines and chemokines; and efficient processing and presentation of antigens to T-cells. As discussed above, TLRs recognize PAMPs including bacterial cell wall components (e.g., bacterial lipoproteins and lipopolysaccharides), bacterial DNA sequences that contain unmethylated CpG residues and bacterial flagellin that act as initiators of the innate immune response and gatekeepers of the adaptive immune response (Medzhitov, R., et al., Cold Springs Harb. Symp. Quant. Biol. 64:429 (1999); Pasare, C., et al., Semin, Immunol 16:23 (2004); Medzhitov, R., et al., Nature 388:394 (1997); Barton, G. M., et al., Curr. Opin. Immunol 14:380 (2002); Bendelac, A., et al., J. Exp. Med. 195:F19 (2002)).

The compositions and proteins of the invention can trigger an immune response to a papillomavirus protein component (e.g., E6, E7, L2 protein) and trigger signal transduction pathways of the innate and adaptive immune system of the subject to thereby stimulate the immune system of a subject to generate antibodies and protective immunity to the papillomavirus protein component of the composition. Thus, stimulation of the immune system of the subject may prevent infection by a papillomavirus, such as HPV, and thereby treat the subject or prevent the subject from disease, illness and, possibly, death.

“Agonist,” as used herein in referring to a TLR, means a molecule that activates a TLR signaling pathway. As discussed above, a TLR signaling pathway is an intracellular signal transduction pathway employed by a particular TLR that can be activated by a TLR ligand or a TLR agonist. Common intracellular pathways are employed by TLRs and include, for example, NF-κB, Jun N-terminal kinase and mitogen-activated protein kinase. The Toll-like Receptor agonist can include at least one member selected from the group consisting of a TLR1 agonist, a TLR2 agonist (e.g., Pam3Cys, Pam2Cys, bacterial lipoprotein), a TLR3 agonist (e.g., dsRNA), a TLR4 agonist (e.g., bacterial lipopolysaccharide), a TLR5 agonist (e.g., a flagellin), a TLR6 agonist, a TLR7 agonist, a TLR8 agonist, a TLR9 agonist (e.g., unmethylated DNA motifs), TLR10 agonist, a TLR11 agonist and a TLR12 agonist. Exemplary suitable Toll-like Receptor agonist components for use in the invention are described, for example, in U.S. application Ser. Nos.: 11/820,148, 11/879,695, 11/714,873, and 11/714,684, the entire teachings of all of which are hereby incorporated by reference in their entirety.

The Toll-like Receptor agonists for use in the methods and compositions of the invention can also be a Toll-like Receptor agonist component that is at least a portion of a Toll-like Receptor agonist, wherein the Toll-like Receptor agonist component includes at least one cysteine residue in a position where a cysteine does not occur in the native Toll-like Receptor agonist, whereby the Toll-like Receptor agonist component activates a Toll-like Receptor.

TLR4 ligands (e.g., TLR4 agonists, such as SEQ ID NOs: 1-48) for use in the compositions and methods of the invention can include at least one member selected from the group consisting of (see, PCT/US 2006/002906/WO 2006/083706; PCT/US 2006/003285/WO 2006/083792; PCT/US 2006/041865; PCT/US 2006/042051).

TLR2 ligands (e.g., TLR2 agonists, such as SEQ ID NOs: 49-88) for use in the compositions and methods of the invention can also include at least one member selected from the group consisting of (see, PCT/US 2006/002906/WO 2006/083706; PCT/US 2006/003285/WO 2006/083792; PCT/US 2006/041865; PCT/US 2006/042051).

The TLR2 ligand (e.g., TLR2 agonist) can also include at least a portion of at least one member selected from the group consisting of flagellin modification protein FlmB of Caulobacter crescentus; Bacterial Type III secretion system protein; invasin protein of Salmonella; Type 4 fimbrial biogenesis protein (PilX) of Pseudomonas; Salmonella SciJ protein; putative integral membrane protein of Streptomyces; membrane protein of Pseudomonas; adhesin of Bordetella pertusis; peptidase B of Vibrio cholerae; virulence sensor protein of Bordetella; putative integral membrane protein of Neisseria meningitidis; fusion of flagellar biosynthesis proteins FliR and FlhB of Clostridium; outer membrane protein (porin) of Acinetobacter; flagellar biosynthesis protein FlhF of Helicobacter; ompA related protein of Xanthomonas; omp2a porin of Brucella; putative porin/fimbrial assembly protein (LHrE) of Salmonella; wbdk of Salmonella; Glycosyltransferase involved in LPS biosynthesis; Salmonella putative permease.

The TLR2 ligand (e.g., TLR agonist) can include at least a portion of at least one member selected from the group consisting of lipoprotein/lipopeptides (a variety of pathogens); peptidoglycan (Gram-positive bacteria); lipoteichoic acid (Gram-positive bacteria); lipoarabinomannan (mycobacteria); a phenol-soluble modulin (Staphylococcus epidermidis); glycoinositolphospholipids (Trypanosoma Cruzi); glycolipids (Treponema maltophilum); porins (Neisseria); zymosan (fungi) and atypical LPS (Leptospira interrogans and Porphyromonas gingivalis).

The TLR2 ligand (e.g., TLR2 agonist, such as SEQ ID NOs: 89-91) can also include at least one member selected from the group consisting of (see, PCT/US 2006/002906/WO 2006/083706; PCT/US 2006/003285/WO 2006/083792; PCT/US 2006/041865; PCT/US 2006/042051).

The TLR2 agonist can include at least a portion of a bacterial lipoprotein (BLP).

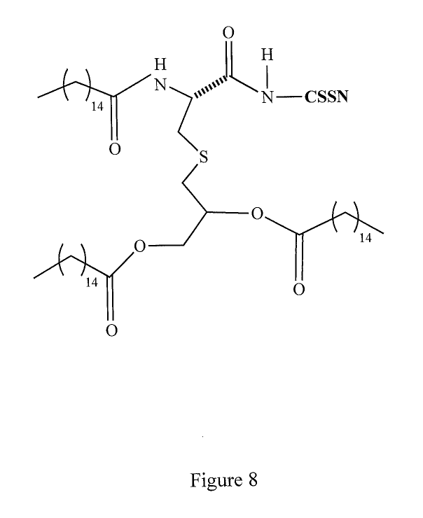

The TLR2 agonist can be a bacterial lipoprotein, such as Pam2Cys (S-[2,3-bis(palmitoyloxy) propyl] cysteine), Pam3Cys ([Palmitoyl]-Cys((RS)-2,3-di(palmitoyloxy)-propyl cysteine) or Pseudomonas aeruginosa OprI lipoprotein (OprI). Exemplary OprI lipoproteins include SEQ ID NO: 92, encoded by SEQ ID NO: 93. An exemplary protein component of an E. coli bacterial lipoprotein for use in the invention described herein is SEQ ID NO: 94 encoded by SEQ ID NO: 95. A bacterial lipoprotein that activates a TLR2 signaling pathway (a TLR2 agonist) is a bacterial protein that includes a palmitoleic acid (Omueti, K. O., et al., J. Biol. Chem. 280: 36616-36625 (2005)). For example, expression of SEQ ID NOS: 93 and 95 in bacterial expression systems (e.g., E. coli) results in the addition of a palmitoleic acid moiety to a cysteine residue of the resulting protein (e.g., SEQ ID NOS: 92 and 94) thereby generating a TLR2 agonist for use in the compositions, fusion proteins and polypeptides of the invention. Production of tripalmitoylated-lipoproteins (also referred to as triacyl-lipoproteins) in bacteria occurs through the addition of a diacylglycerol group to the sulfhydryl group of a cysteine (e.g., cysteine 21 of SEQ ID NO: 94) followed by cleavage of the signal sequence and addition of a third acyl chain to the free N-terminal group of the same cysteine (e.g., cysteine 21 of SEQ ID NO: 94) (Sankaran, K., et al., J. Biol. Chem. 269:19706 (1994)), to generate a tripalmitylated peptide (a TLR2 agonist) as shown, for example, in FIG. 8.

The Toll-like Receptor agonist in the compositions of the invention can further include at least one cysteine residue at the terminal amino acid of the amino-terminus and/or the terminal amino acid of the carboxy-terminus of the Toll-like Receptor agonist. For example, GGKLS (SEQ ID NO: 96) can further include at least one cysteine residue in a peptide bond to the amino-terminal glycine residue and/or at least one cysteine residue in a peptide bond to the carboxy-terminal glycine residue (e.g., FCGLG, such as SEQ ID NO: 97) can further include at least one cysteine residue in a peptide bond to the amino-terminal lysine residue and/or at least one cysteine residue in a peptide bond to the carboxy-terminal glycine residue; sequences can further include at least one cysteine residue in a peptide bond to the amino-terminal glutamic acid residue and/or at least one cysteine residue in a peptide bond to the carboxy-terminal proline residue. Cysteine residues can also be included at the carboxy or amino-terminus of a TLR5 agonist, such as a flagellin, as discussed below.

TLR agonists can also include SEQ ID NOs: 195-207.

In an embodiment, the TLR agonist is a TLR5 agonist, such as flagellin. The flagellin in the compositions and methods described herein can be at least a portion of a S. typhimurium flagellin (GenBank Accession Number AF045151); at least a portion of the S. typhimurium flagellin selected from the group consisting of SEQ ID NO: 98, SEQ ID NO: 99, SEQ ID NO: 100 and SEQ ID NO: 101; at least a portion of an S. muenchen flagellin (GenBank Accession Number AB028476) that includes at least a portion of SEQ ID NO: 102 and SEQ ID NO: 103; at least a portion of P. aeruginosa flagellin that includes at least a portion of SEQ ID NO: 104; at least a portion of a Listeria monocytogenes flagellin that includes at least a portion of SEQ ID NO: 105; at least a portion of an E. coli flagellin that includes at least a portion of SEQ ID NO: 106 and SEQ ID NO: 107; at least a portion of a Yersinia flagellin; and at least a portion of a Campylobacter flagellin.

The flagellin employed in the compositions of the invention can also include the polypeptides of SEQ ID NO: 98, SEQ ID NO: 101, SEQ ID NO: 102 and SEQ ID NO: 106; at least a portion of SEQ ID NO: 98, at least a portion of SEQ ID NO: 101, at least a portion of SEQ ID NO: 102, at least a portion of SEQ ID NO: 193 and at least a portion of SEQ ID NO: 106; and a polypeptide encoded by SEQ ID NO: 108, SEQ ID NO: 109, SEQ ID NO: 110 and SEQ ID NO: 111; or at least a portion of a polypeptide encoded by SEQ ID NO: 108, SEQ ID NO: 109, SEQ ID NO: 110, SEQ ID NO: 111 and SEQ ID NO: 194. Exemplary flagellin constructs for use in the invention are described, for example, in U.S. application Ser. Nos. 11/820,148; 11/714,873 and 11/714,684, the teachings of all of which are hereby incorporated by reference in their entirety.

The flagellin employed in the compositions and method of the invention can lack at least a portion of a hinge region. Hinge regions are the hypervariable regions of a flagellin. Hinge regions of a flagellin are also referred to herein as “D3 domain or region,” “propeller domain or region,” “hypervariable domain or region” and “variable domain or region.” “Lack” of a hinge region of a flagellin, means that at least one amino acid or at least one nucleic acid codon encoding at least one amino acid that comprises the hinge region of a flagellin is absent in the flagellin. Examples of hinge regions include amino acids 176-415 of SEQ ID NO: 98, which are encoded by nucleic acids 528-1245 of SEQ ID NO: 108; amino acids 174-422 of SEQ ID NO: 106, which are encoded by nucleic acids 522-1266 of SEQ ID NO: 111; or amino acids 173-464 of SEQ ID NO: 102, which are encoded by nucleic acids 519-1392 of SEQ ID NO: 110. Thus, if amino acids 176-415 were absent from the flagellin of SEQ ID NO: 98, the flagellin would lack a hinge region. A flagellin lacking at least a portion of a hinge region is also referred to herein as a “truncated version” of a flagellin.

“At least a portion of a hinge region,” as used herein, refers to any part of the hinge region of the flagellin, or the entirety of the hinge region. “At least a portion of a hinge region” is also referred to herein as a “fragment of a hinge region.” At least a portion of the hinge region of fljB/STF2 can be, for example, amino acids 200-300 of SEQ ID NO: 98. Thus, if amino acids 200-300 were absent from SEQ ID NO: 98, the resulting amino acid sequence of STF2 would lack at least a portion of a hinge region.

Alternatively, at least a portion of a naturally occurring flagellin can be replaced with at least a portion of an artificial hinge region. “Naturally occurring,” in reference to a flagellin amino acid sequence, means the amino acid sequence present in the native flagellin (e.g., S. typhimurium flagellin, S. muenchin flagellin, E. coli flagellin). The naturally occurring hinge region is the hinge region that is present in the native flagellin. For example, amino acids 176-415 of SEQ ID NO: 98, amino acids 174-422 of SEQ ID NO: 106 and amino acids 173-464 of SEQ ID NO: 102, are the amino acids corresponding to the natural hinge region of STF2, E. coli fliC and S. muenchen flagellins, fliC, respectively. “Artificial,” as used herein in reference to a hinge region of a flagellin, means a hinge region that is inserted in the native flagellin in any region of the flagellin that contains or contained the native hinge region.

The hinge region of a flagellin can be deleted and replaced with at least a portion of an HPV protein component (e.g., E6, E7, L2).

An artificial hinge region may be employed in a flagellin that lacks at least a portion of a hinge region, which may facilitate interaction of the carboxy- and amino-terminus of the flagellin for binding to TLR5 and, thus, activation of the TLR5 innate signal transduction pathway. A flagellin lacking at least a portion of a hinge region is designated by the name of the flagellin followed by a “Δ.” For example, an STF2 (e.g., SEQ ID NO: 98) that lacks at least a portion of a hinge region is referenced to as “STF2Δ” or “fljB/STF2Δ” (e.g., SEQ ID NO: 101).

The flagellin for use in the methods and compositions of the invention can be a at least a portion of a flagellin, wherein the flagellin component includes at least one cysteine residue and whereby the flagellin component activates a Toll-like Receptor 5; a flagellin component that is at least a portion of a flagellin, wherein at least one lysine of the flagellin component has been substituted with at least one arginine, whereby the flagellin component activates a Toll-like Receptor 5; a flagellin component that is at least a portion of a flagellin, wherein at least one lysine of the flagellin component has been substituted with at least one serine residue, whereby the flagellin component activates a Toll-like Receptor 5; a flagellin component that is at least a portion of a flagellin, wherein at least one lysine of the flagellin component has been substituted with at least one histidine residue, whereby the flagellin component activates a Toll-like Receptor 5, as described herein.

The Toll-like Receptor agonist and the papillomavirus proteins employed in the compositions and methods of the invention (e.g., papillomavirus suppressor binding protein, papillomavirus capsid protein, papillomavirus transforming protein) can be components of a fusion protein.

“Fusion proteins,” as used herein, refers to the joining of two components (also referred to herein as “fused” or :linked”) (e.g., a Toll-like receptor agonist and at least a portion of an HPV antigen, such as at least a portion of at least one member selected from the group consisting of a papillomavirus suppressor binding protein, a papillomavirus capsid protein, a papillomavirus transforming protein). Fusion proteins of the invention can be generated from at least two similar or distinct components. Fusion proteins of the invention can be generated by recombinant DNA technologies or by chemical conjugation of the components of the fusion protein. Recombinant DNA technologies and chemical conjugation techniques are well established procedures and known to one of skill in the art. Exemplary techniques to generate fusion proteins that include Toll-like Receptor agonists are described herein and in U.S. application Ser. Nos. 11/714,684 and 11/714,873, the teachings of both of which are hereby incorporated by reference in their entirety.

Exemplary fusion proteins of the invention and nucleic acids encoding the fusion proteins include the nucleic acid sequence (SEQ ID NO: 230) and encoded amino acid sequence (SEQ ID NO: 231) of an E6 protein for use in the invention (STF2.HPV16 E6CTLHis₆); the nucleic acid sequence (SEQ ID NO: 232) and encoded amino acid sequence (SEQ ID NO: 233) of a fusion protein for use in the invention (STF2.HPV16 4xE6CTL His₆); the nucleic acid sequence (SEQ ID NO: 234) of a fusion protein of the invention (STF2.HPV16 E7; SEQ ID NO: 208); the nucleic acid sequence (SEQ ID NO: 235) of a fusion protein of the invention (STF2Δ.HPV16 E7; SEQ ID NO: 212); the nucleic acid sequence (SEQ ID NO: 236) of an E7 protein for use in the invention (HPV16 E7; SEQ ID NO: 151); the nucleic acid sequence (SEQ ID NO: 237) of an E7 protein for use in the invention (HPV16 E7His₆); the nucleic acid sequence (SEQ ID NO: 238) and encoded amino acid sequence (SEQ ID NO: 239) of a fusion protein of the invention (STF2.HPV16 E7CTLHis₆); the nucleic acid sequence (SEQ ID NO: 240) and encoded amino acid sequence (SEQ ID NO: 241) of a fusion protein of the invention (STF2.HPV16 4xE7CTLHis₆); the nucleic acid sequence (SEQ ID NO: 242) of a fusion protein of the invention (STF2.HPV16 E6E7; SEQ ID NO: 210); the nucleic acid sequence (SEQ ID NO: 243) of a fusion protein of the invention (STF2Δ.HPV16 E6E7; SEQ ID NO: 214); the nucleic acid sequence (SEQ ID NO: 244) of an E6E7 protein for use in the invention (HPV16 E6E7; SEQ ID NO: 221); the nucleic acid sequence (SEQ ID NO: 245) of an E6E7 protein for use in the invention (HPV16 E6E7His₆; SEQ ID NO: 220); the nucleic acid sequence (SEQ ID NO: 246) of a fusion protein of the invention (STF2.HPV16 L2; SEQ ID NO: 211); the nucleic acid sequence (SEQ ID NO: 247) of a fusion protein of the invention (STF2Δ.HPV16 L2; SEQ ID NO: 215); the nucleic acid sequence (SEQ ID NO: 248) of an L2 protein for use in the invention (HPV16 L2; SEQ ID NO: 166); and the nucleic acid sequence (SEQ ID NO: 249) of an L2 protein for use in the invention (HPV16 L2His₆; SEQ ID NO: 222).

In an embodiment, a carboxy-terminus of the papillomavirus component is fused (also referred to herein as “linked”) to an amino terminus of the flagellin component of the protein. In another embodiment, an amino-terminus of the papillomavirus protein component is fused to a carboxy-terminus of the flagellin component of the protein.

Fusion proteins of the invention can be designated by the components of the fusion proteins separated by a “.”. For example, “STF2.E6” refers to a protein comprising one STF2 protein and one E6 protein; and “STF2Δ.E6” refers to a fusion protein comprising one STF2 protein without the hinge region and E6 protein. Exemplary fusion proteins of the invention include SEQ ID NOS: 208-215.

Proteins of the invention can include, for example, two, three, four, five, six or more Toll-like Receptor agonists (e.g., flagellin) and two, three, four, five, six or more HPV proteins. When two or more TLR agonists and/or two or more proteins comprise proteins of the invention, they are also referred to as “multimers.” For example, a multimer of an E6 protein can be four E6 sequences, which is referred to herein as 4xE6.

The proteins of the invention can further include a linker between at least one component of the protein (e.g., an E6, E7, L2 protein) and at least one other component of the protein (e.g., flagellin component) of the composition, a linker (e.g., an amino acid linker) between at least two of similar components of the protein (e.g., an E6, E7, L2 protein) or any combination thereof. The linker can be between the papillomavirus component and Toll-like Receptor agonist component of a fusion protein. “Linker,” as used herein in reference to a protein of the invention, refers to a connector between components of the protein in a manner that the components of the protein are not directly joined. For example, one part of the protein (e.g., flagellin component) can be linked to a distinct part (e.g., E6, E7, L2 component) of the protein. Likewise, at least two or more similar or like components of the protein can be linked (e.g., two flagellin components can further include a linker between each flagellin component, or two papillomavirus protein (e.g., E6, E7, L2 protein) components can further include a linker between each papillomavirus protein).

Additionally, or alternatively, the proteins of the invention can include a combination of a linker between distinct components of the protein and similar or like components of the protein. For example, a protein can comprise at least two TLR agonists that further includes a linker between, for example, two or more flagellin; at least two papillomavirus protein components that further include a linker between them; a linker between one component of the protein (e.g., flagellin) and another distinct component of the protein (e.g., papillomavirus protein component), or any combination thereof.

The linker can be an amino acid linker. The amino acid linker can include synthetic or naturally occurring amino acid residues. The amino acid linker employed in the proteins of the invention can include at least one member selected from the group consisting of a lysine residue, a glutamic acid residue, a serine residue and an arginine residue.

The Toll-like Receptor agonist of the proteins of the invention can be fused to a carboxy-terminus, the amino-terminus or both the carboxy- and amino-terminus of the HPV protein component.

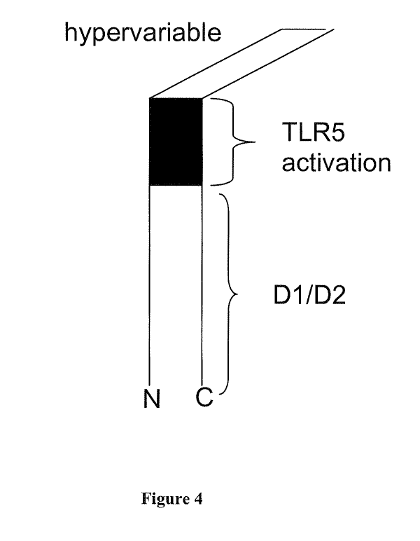

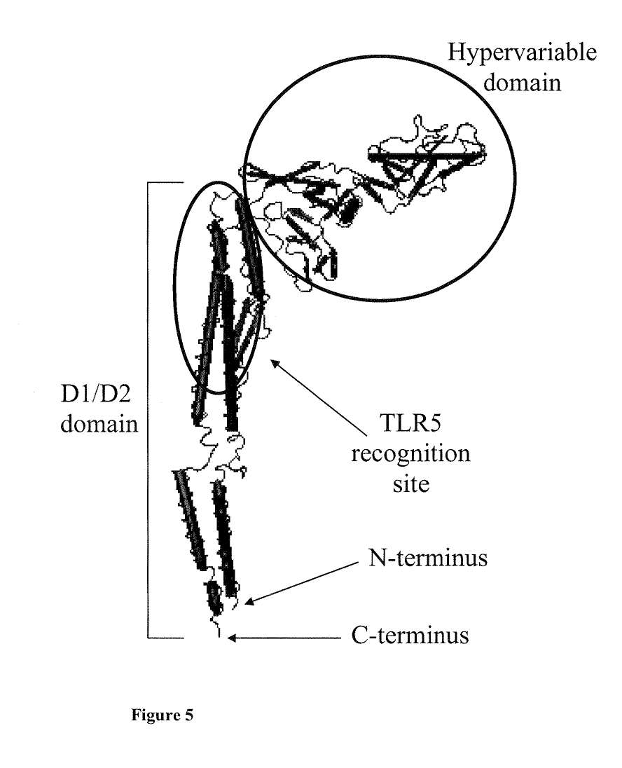

Proteins can be generated by fusing the HPV protein to at least one of four regions (Regions 1, 2, 3 and 4) of flagellin, which have been identified based on the crystal structure of flagellin (PDB:1UCU) (see, for example, FIGS. 4 and 5). Region 1 is also referred to as Domain O or DO. Region 2 is also referred to as Domain 1 or D1. Region 3 is also referred to as D2. Region 4 is also referred to as D3.

Region 1 is TIAL (SEQ ID NO: 112) . . . - . . . GLG (194-211 of SEQ ID NO: 99). The corresponding residues for Salmonella typhimurium fljB construct are TTLD (SEQ ID NO: 114) . . . - . . . GTN (196-216 of SEQ ID NO: 113). This region is an extended peptide sitting in a groove of two beta strands (GTDQKID (SEQ ID NO: 115) and NGEVTL (SEQ ID NO: 116) of (SEQ ID NO: 99).

Region 2 of the Salmonella flagellin is a small loop GTG (238-240 of SEQ ID NO: 99) in 1UCU structure (see, for example, FIGS. 4 and 5). The corresponding loop in Salmonella fljB is GADAA (SEQ ID NO: 117) (244-248 of SEQ ID NO: 113).

Region 3 is a bigger loop that resides on the opposite side of the Region 1 peptide (see, for example, FIGS. 4 and 5). This loop can be simultaneously substituted together with region 1 to create a double copy of the antigen (e.g., an E6, E7, L2 protein portion). The loop starts from AGGA (SEQ ID NO: 118) and ends at PATA (SEQ ID NO: 119) (259-274 of SEQ ID NO: 99). The corresponding Salmonella fljB sequence is AAGA (SEQ ID NO: 126 . . . - . . . ATTK (SEQ ID NO: 120) (266-281 of SEQ ID NO: 113). The sequence AGATKTTMPAGA (SEQ ID NO: 121) (267-278 of SEQ ID NO: 113) can be replaced with an papillomavirus protein.

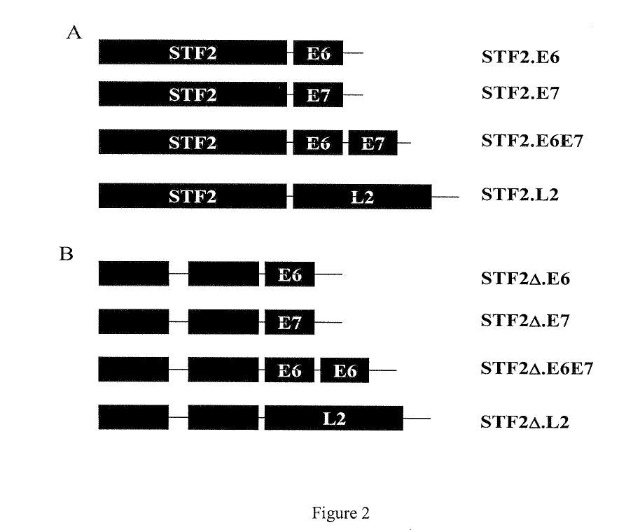

Region 4 is the loop (GVTGT (SEQ ID NO: 229)) connecting a short α-helix (TEAKAALTAA (SEQ ID NO: 123)) and a β-strand (ASVVKMSYTDN (SEQ ID NO: 124) SEQ ID NO: 99 (see FIG. 2). The corresponding loop in Salmonella fljB is a longer loop VDATDANGA (SEQ ID NO: 125 (307-315 of SEQ ID NO: 113). An HPV protein, including an HPV antigen, can be inserted into or replace this region.

The compositions of the invention include at least a portion of a papillomavirus. Papillomaviruses are small, non-enveloped icosahedral particles about 52-55 nm diameter. There are 72 capsomers (60 hexameric and 12 pentameric) arranged on a T=7 lattice. The papillomavirus genome includes circular, double-stranded DNA about 8 kb in size, which is associated with cellular histones to form a chromatin-like substance. At least 12 different HPV genomes have been sequenced, all of which share a similar genetic organization.

Individual isolates of papillomaviruses are highly species specific. Papillomaviruses infect the basal cells of the dermal layer of the skin, where early gene expression can be detected. Late gene expression, expression of structural proteins and vegetative DNA synthesis is restricted to terminally differentiated cells of the epidermis, which may be associated with cellular differentiation and viral gene expression.

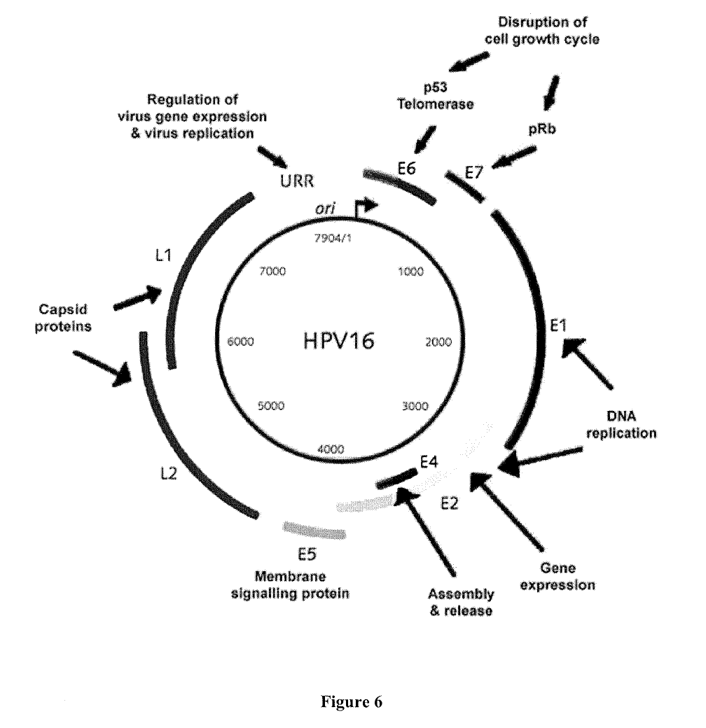

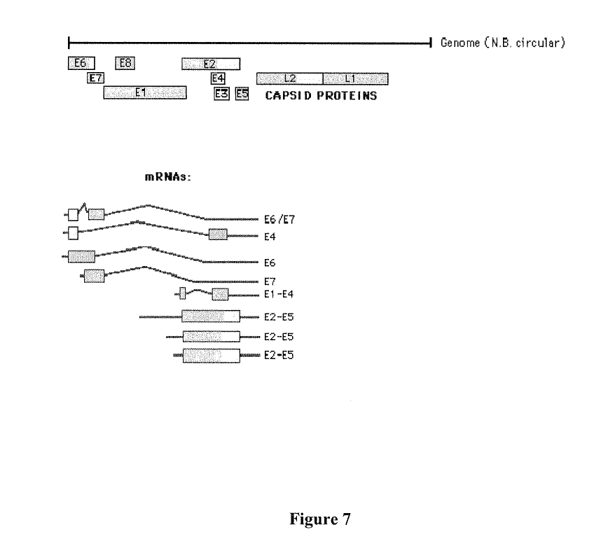

Genes of the papillomavirus include the E1 gene, the E2 gene, the E3 gene, E4 gene, E5 gene, E6 gene, E7 gene, E8 gene, L1 gene and L2 genes (FIGS. 6 and 7). The E1 gene is a DNA-dependent ATPase, ATP dependent helicase, that permits unwinding of the viral genome and act as an elongation factor for DNA replication.

The E2 gene is responsible for recognition and binding of origin of replication and exists in two forms—a full length form (transcriptional transactivator) and a truncated form (transcriptional repressor).

The E4 gene is a late expression. The C terminal of the E4 gene product binds an intermediate filament, allowing release of virus-like particles. The E4 gene is also involved in transformation of the host cell by deregulation of the host cell mitogenic signaling pathway.

The E5 gene participates in obstruction of growth suppression mechanisms, such as EGF receptor, activates mitogenic signaling pathways by transcription factors, c-Jun and c-Fos, which may be important in ubiquitin pathway degradation of p53 complex by the E6 gene.

The E6 gene participates in transformation of the host cell by binding p53 tumor suppressor protein.

The E7 gene is a transforming protein, which binds to pRB/p107.

The L1 gene is a major capsid protein, which can form virus-like particles.

The L2 gene is a minor capsid protein, which may participate in DNA packaging protein.

Transcription has been studied in detail by transfection of cloned papillomavirus DNA into cells. Only one strand of the genome is transcribed. Two classes of proteins are produced—(1) early proteins: non-structural regulatory proteins, including trans-acting transcriptional regulators (E2, E7); and (2) late proteins: structural proteins L1 and L2.

Transformation in HPV can include the binding of E2 to the early promoter and decrease expression of E6/E7 and loss of E2 at the first stage in transformation. E6 binds to p53 by a cellular protein (p100) and targets it for degradation by the ubiquitin pathway. E7 binds pRB and prevents phosphorylation, which generally would result in apoptosis. However, E6 and E7 interact with a number of cellular proteins that influence the outcome of infection.

The HPV E7 proteins are small (HPV16 E7 about 98 amino acids), zinc binding phosphoproteins which are in the nucleus. They are structurally and functionally similar to the E1A protein of subgenus C adenoviruses. The first about 16 amino-terminal amino acids of HPV16 E7 contain a region homologous to a segment of the conserved region 1 (CR1) of the E1A protein of subgenus C adenoviruses. The next domain, up to about amino acid 37, is homologous to the entire region 2 (CR2) of E1A. Genetic studies have established that these domains are required for cell transformation in vitro, suggesting similarities in the mechanism of transformation by these viruses. The CR2 homology region contains the LXCXE (SEQ ID NO: 224) motif (residues 22-26) involved in binding to the tumor suppressor protein pRb. This sequence is also present in SV40 and polyoma large T antigens. The high risk HPV E7 proteins (e.g., HPV16 and HPV18) have about A ten-fold higher affinity for pRb protein than the low risk HPV E7 proteins (e.g., HPV6). Association of the E7 protein with pRb promotes cell proliferation by the same mechanism as the E1A proteins of adenoviruses and SV40 large T antigen. E7 may promote degradation of Rb family proteins rather than simply inhibiting their function by complex formation. The CR2 region also contains the casein kinase II phosphorylation site (residues 31 and 32). The remaining 61 amino acids of E7 protein have very little similarity to E1A, however a sequence CXXC (SEQ ID NO: 192) involved in zinc binding is present in both proteins. The E7 protein contains two of these motifs which mediate dimerization of the protein. Mutation in one of the two zinc binding motifs destroys transforming activity, although this mutant is able to associate with Rb protein. Thus, dimerization may be important for the transforming activity of E7.

The HPV E6 are small basic proteins (HPV16 E6 about 151 amino acids) which are localized to the nuclear matrix and non-nuclear membrane fraction. They contain four cysteine motifs, which are thought to be involved in zinc binding. E6 encoded by high risk HPVs associates with the wild type p53 tumor suppressor protein. For association with p53, the E6 protein requires a cellular protein of about 100 kDa, termed E6-associated protein (E6-AP). Like SV40 large T antigen and Ad5 E1B 58 kDa, E6 proteins of high risk HPVs abrogate the ability of wild type p53 to activate transcription. However, the mechanism of E6 action is different than that of SV40 large T and the E1B protein since it involves degradation of p53. The E6-dependent degradation of p53 may occur through the cellular ubiquitin proteolysis pathway. (O'Brien, P. M., et al., Trends in Microbiol. 11:300-305 (2003)).

The genome of the papillomavirus can be replicated as a multicopy nuclear plasmid (episome). Two mechanisms are involved in genome replication—phase replication and vegetative replication.

Phase replication occurs in cells in the lower levels of the dermis. Initially, the virus DNA is amplified to between about 50 to about 400 copies/diploid genome then replicates once per cell division, the copy number/cell remaining constant. The E1 protein is involved in this phase of replication.

Vegetative replication occurs in terminally differentiated cells in the epidermis. In terminally differentiated cells (or growth-arrested cells in culture) control of copy number appears to be lost and the DNA is amplified up to very high copy numbers. The papillomavirus is shed from epidermal cells when these are sloughed off and is transmitted by direct contact (especially genital warts) and indirect contact.

The risk of cancer from HPV infection and site of infection from HPF can vary with type of HPV as shown in Tables 1 and 2.

TABLE 1

Site of infection

| Risk of Cancer | Skin | Genital |
|---|---|---|
| High risk | HPV5 | HPV16 |
| (Flat lesions) | HPV8 | HPV18 |
| Low risk | HPV1 | HPV6 |
| (Warty lesions) | HPV2 | HPV11 |

TABLE 2
Epidemiological classification of HPV types

| | |
|---|---|
| Established High Risk | 16, 18, 31, 33, 35, 39, 45, 51, 52, 56, 58, 59 |
| Probably High Risk | 26, 53, 66, 68, 73, 82 |
| Established Low Risk | 6, 11, 40, 42, 43, 44, 54, 61, 70, 72, 81, CP6108 |

Human papillomaviruses (HPVs) are viruses that cause benign and malignant tumors. More than 100 HPV genotypes have been identified and about 30 infect the genital tract (Table 1). The latter have been classified as either “high-risk,” those more likely to cause dysplasia and cancer, or “low risk,” which more often cause benign lesions such as warts. HPV16 alone is responsible for about 25% of all infections, 50% of high-grade cervical dysplasia and 60% of cervical cancer. It is also responsible for about 60% of anal dysplasia and anal cancer. HPV16 and HPV18 are identified as high risk HPVs which are mainly responsible for HPV correlated human cancer. HPV6 and HPV11 cause almost all cases of genital warts.

In high risk HPV infected cells, the oncoproteins E6 and E7 are usually co-expressed, with abrogates negative growth regulatory signaling pathways of the host cell through interaction with p53 and pRB tumor suppressor proteins. As a result, the proliferation of high-risk HPV infected cells becomes de-regulated, and consequently transformation ensues. Both of these proteins are required to maintain the transformed states of HPV-induced neoplastic cells. The fusion between the flagellin and the oncoprotein may potentiate the immune response by activating the innate immune system which is coupled to the adaptive immune system.

“Tumor suppressor binding protein,” as used in reference to a papillomavirus, refers to a protein that associates with at least one protein that binds at least one gene that reduces the likelihood or ability of a cell to proliferate uncontrollably. A consequence of binding to the tumor suppressor protein can include uncontrolled cell proliferation, including tumor and cancer formation.

“Activates,” when referring to a Toll-like Receptor (TLR), means that the component (e.g., a flagellin component or a Toll-like Receptor agonist component) or the protein of the invention stimulates a response associated with a TLR. For example, bacterial flagellin activates TLR5 and host inflammatory responses (Smith, K. D., et al., Nature Immunology 4:1247-1253 (2003)). Bacterial lipopeptide activates TLR1; Pam3Cys, Pam2Cys activate TLR2; dsRNA activates TLR3; LBS (LPS-binding protein) and LPS (lipopolysaccharide) activate TLR4; imidazoquinolines (anti-viral compounds and ssRNA) activate TLR7; and bacterial DNA (CpG DNA) activates TLR9. TLR1 and TLR6 require heterodimerization with TLR2 to recognize ligands (e.g., TLR agonists, TLR antagonists). TLR1/2 are activated by triacyl lipoprotein (or a lipopeptide, such as Pam3Cys), whereas TLR6/2 are activated by diacyl lipoproteins (e g., Pam2Cys), although there may be some cross-recognition. In addition to the natural ligands, synthetic small molecules including the imidazoquinolines, with subclasses that are specific for TLR7 or TLR8 can activate both TLR7 and TLR8. There are also synthetic analogs of LPS that activate TLR4, such as monophosphoryl lipid A [MPL].

TLR activation can result in signaling through MyD88 and NF-κB. There is some evidence that different TLRs induce different immune outcomes. For example, Hirschfeld, et al. Infect Immun 69:1477-1482 (2001)) and Re, et al. J Biol Chem 276:37692-37699 (2001) demonstrated that TLR2 and TLR4 activate different gene expression patterns in dendritic cells. Pulendran, et al J Immunol 167:5067-5076 (2001)) demonstrated that these divergent gene expression patterns were recapitulated at the protein level in an antigen-specific response, when lipopolysaccharides that signal through TLR2 or TLR4 were used to guide the response (TLR4 favored a Th1-like response with abundant IFNγ secretion, while TLR2 favored a Th2-line response with abundant IL-5, IL-10, and IL-13 with lower IFNγ levels). There is redundancy in the outcome of signaling through different TLRs.

Activation of TLRs can result in increased effector cell activity that can be detected, for example, by measuring IFNγ-secreting CD8+ cells (e.g., cytotoxic T-cell activity); increased antibody responses that can be detected by, for example, ELISA, virus neutralization, and flow cytometry (Schnare, M., et al., Nat Immunol 2:947 (2001); Alexopoulou, L., et al., Nat Med 8:878 (2002); Pasare, C., et al., Science 299:1033 (2003); Napolitani, G., et al., Nat Immunol 6:769 (2005); and Applequist, S. E., et al., J Immunol 175:3882 (2005)).

The Toll-like Receptor agonist of the compositions of the invention can include a Toll-like Receptor 5 agonist. The Toll-like Receptor 5 agonist can include at least a portion of at least one flagellin (e.g., SEQ ID NO: 193, SEQ ID NO: 135) The flagellin includes at least one member selected from the group consisting of a Salmonella typhimurium flagellin (e.g., SEQ ID NO: 1), an E. coli flagellin, a S. muenchen flagellin, a Yersinia flagellin, a P. aeruginosa flagellin and a L. monocytogenes flagellin. The flagellin can lack at least a portion of a hinge region (e.g., SEQ ID NO: 135).

The papillomavirus tumor suppressor binding protein can include a human papillomavirus tumor suppressor binding protein, such as at least a portion of a p53 tumor suppressor binding protein (e.g., at least a portion of an E6 protein). Exemplary E6 proteins, the nucleic acids encoding the E6 proteins and Gen Bank Accession Numbers for use include SEQ ID NOs: 136 and 137-150. For example, the amino acid sequences (SEQ ID NOs: 137 and 139) of an HPV2 E6 protein (GenBank Accession No: NP_{—}040305, SEQ ID NO: 137 and AB014920, SEQ ID NO: 139) and the corresponding nucleic acid sequences (SEQ ID NOs: 138 and 140); the amino acid sequence (GenBank Accession No: BAA42817, SEQ ID NO: 141 and GenBank Accession No: AAF00064, SEQ ID NO: 143) and corresponding nucleic acid sequences (SEQ ID NOs: 142 and 144) of an E6 protein of HPV5 for use in the compositions of the invention; the amino acid sequence (GenBank Accession No: AAD47251, SEQ ID NO: 145 and GenBank Accession No: P04019, SEQ ID NO: 147) and the corresponding nucleic acid sequences (SEQ ID NOS: 146 and 148) of an E6 protein of HPV 11 for use in the compositions of the invention; and the amino acid sequence (GenBank Accession No: CAA00542, SEQ ID NO: 149) and corresponding nucleic acid sequence (SEQ ID NO: 150) of an E6 protein of HPV18.

The compositions that include at least at least one protein that includes at least a portion of at least one Toll-like Receptor agonist and at least a portion of at least one papillomavirus tumor suppressor binding protein, wherein the protein activates a Toll-like Receptor can further include at least a portion of at least one member selected from the group consisting of a papillomavirus transforming protein, such as papillomavirus transforming protein that binds to at least one retinoblastoma protein (pRB), including at least one member selected from the group consisting of a pRB protein and a p107 protein, such as at least a portion of an E7 protein and a papillomavirus capsid protein. Exemplary E7 proteins, nucleic acids encoding the E7 proteins and Gen Bank Accession Numbers for an E7 protein include SEQ ID NOs: 151 and 152-164. For example, the amino acid sequence (GenBank Accession No: NP_{—}040307, SEQ ID NO: 152; GenBank Accession No: AB014921, SEQ ID NO: 154; GenBank Accession No: CAA52694, SEQ ID NO: 156) and the corresponding nucleic acid sequences (SEQ ID NOs: 153, 155 and 157) of an E7 protein of HPV1, 2 and 5; the amino acid sequence (GenBank Accession No: AAF00065, SEQ ID NO: 158; GenBank Accession No: P06430, SEQ ID NO: 160; GenBank Accession No: P04020, SEQ ID NO: 161; and GenBank Accession No: CAA00543, SEQ ID NO: 163) and the corresponding nucleic acid sequences for SEQ ID NOS: 158, 161 and 163, respectively (SEQ ID NOs: 159, 162 and 164) of an E7 protein of HPV6, 8, 11 and 18.

Genetic evidence from retinoblastoma patients and experiments describing the mechanism of cellular transformation by the DNA tumor viruses have defined a central role for the retinoblastoma protein (pRB) family of tumor suppressors in the normal regulation of the eukaryotic cell cycle. These proteins include pRB, p107 and p130, which act in a cell cycle-dependent manner to regulate the activity of a number of important cellular transcription factors, such as the E2F-family, which in turn regulate expression of genes whose products are important for cell cycle progression. In addition, inhibition of E2F activity by the pRB family proteins is required for cell cycle exit after terminal differentiation or nutrient depletion. The loss of functional pRB, due to mutation of both RB1 alleles, results in deregulated E2F activity and a predisposition to specific malignancies. Similarly, inactivation of the pRB family by the transforming proteins of the DNA tumor viruses overcomes cellular quiescence and prevents terminal differentiation by blocking the interaction of pRB, p107, and p130 with the E2F proteins, leading to cell cycle progression and, ultimately, cellular transformation. The pRB family of proteins may be negative cell cycle regulators and the E2F family of transcription factors may be central components in the cell cycle machinery (See, for example, Inman, G. J., et al., J. Gen. Virol. 76:2141-2149 (1995); Sidle, A., et al., Critical Reviews in Biochemistry and Molecular Biology, 31: 237-271 (1996); Robanus-Maandag, E., et al., Genes & Development 12:1599-1609 (1998); Classon, M., et al., PNAS 97:10820-25 (2000); Liu, S-L., et al., Oncogene 19:3352-3362 (2000)).

A papillomavirus capsid protein (also referred to as “protein coat”) includes the protein components that surround the nucleic acid of the papillomavirus and its associated protein core. The papillomavirus capside protein can include at least a portion of at least one member selected from the group consisting of at least one papillomavirus major capsid protein and at least one papillomavirus minor capsid protein (e.g., an L2 protein, such as at least a portion of SEQ ID NO: 166). Exemplary L2 proteins for use in the invention include the amino acid (GenBank Accession No: CAA00832, SEQ ID NO: 167) and corresponding nucleic acid sequence (SEQ ID NO: 168) of an L2 protein of HPV1; the amino acid sequence (GenBank Accession No: AAY86489, SEQ ID NO: 169) and corresponding nucleic acid sequence (SEQ ID NO: 170) of an L2 protein of HPV2; the amino acid sequence (GenBank Accession No: AAY86491, SEQ ID NO: 171) and corresponding nucleic acid sequence (SEQ ID NO: 172) of an L2 protein of HPV5; the amino acid sequence (GenBank Accession No: AAF00067, SEQ ID NO: 173) and corresponding nucleic acid sequence (SEQ ID NO: 174) of an L2 protein of HPV6; the amino acid sequence (GenBank Accession No: P06419, SEQ ID NO: 175) of an L2 protein of HPV8; the amino acid sequence (GenBank Accession No: P04013, SEQ ID NO: 176) and corresponding nucleic acid sequence (SEQ ID NO: 177) of an L2 protein of HPV11; the amino acid sequence (GenBank Accession No: AAP20600, SEQ ID NO: 178) and corresponding nucleic acid sequence (SEQ ID NO: 179) of an L2 protein of an HPV18; and the amino acid sequence (GenBank Accession No: AAC80445, SEQ ID NO: 184) and corresponding nucleic acid sequence (SEQ ID NO: 185) of an L2 protein of HPV6.

The papillovirus capsid protein can include at least a portion of a major capsid protein (e.g., an L1 protein) or at least a portion of a minor capsid protein (e.g., an L2 protein). Exemplary L2 proteins, nucleic acids encoding the L2 proteins and Gen Bank Accession numbers include SEQ ID NOs: 166 and 167-179. Exemplary L1 proteins, nucleic acids encoding the L1 protein and Gen Bank Accession Numbers include SEQ ID NOs: 180-191. Exemplary L1 proteins for use in the invention include the amino acid sequence (GenBank Accession No: AAY86492, SEQ ID NO: 182) and corresponding nucleic acid sequence (SEQ ID NO: 183) of an L1 protein of HPV5; the amino acid sequence (GenBank Accession No: AAC80445, SEQ ID NO: 184) and corresponding nucleic acid sequence (SEQ ID NO: 185) of an L2 protein of HPV6; the amino acid sequence (GenBank Accession No: P06417, SEQ ID NO: 186; and GenBank Accession No: AAK21269, SEQ ID NO: 187) of an L1 protein of HPV8 and 11, respectively; the amino acid sequence (GenBank Accession No: AAY57806, SEQ ID NO: 188) and corresponding nucleic acid sequence (SEQ ID NO: 189) of an L1 protein of HPV16; and the amino acid sequence (GenBank Accession No: AAW58852, SEQ ID NO: 190) and corresponding nucleic acid sequence (SEQ ID NO: 225) of an L1 protein of HPV18.

The papillomavirus capsid protein can be a human papillomavirus capsid protein. The papillomavirus capsid protein can include at least one member selected from the group consisting of an HPV1 papillomavirus capsid protein, an HPV2 papillomavirus capsid protein, an HPV5 papillomavirus capsid protein, an HPV6 papillomavirus capsid protein, an HPV8 papillomavirus capsid protein, an HPV11 papillomavirus capsid protein, an HPV16 papillomavirus capsid protein and an HPV18 papillomavirus capsid protein.

The papillomavirus transforming protein can include a human papillomavirus transforming protein. The papillomavirus transforming protein can include at least one member selected from the group consisting of an HPV1 papillomavirus transforming protein, an HPV2 papillomavirus transforming protein, an HPV5 papillomavirus transforming protein, an HPV6 papillomavirus transforming protein, an HPV8 papillomavirus transforming protein, an HPV11 papillomavirus transforming protein, an HPV16 papillomavirus transforming protein and an HPV18 papillomavirus transforming protein.

The papillomavirus tumor suppressor binding protein can include at least one member selected from the group consisting of an HPV1 papillomavirus tumor suppressor binding protein, an HPV2 papillomavirus tumor suppressor binding protein, an HPV5 papillomavirus tumor suppressor binding protein, an HPV6 papillomavirus tumor suppressor binding protein, an HPV8 papillomavirus tumor suppressor binding protein, an HPV11 papillomavirus tumor suppressor binding protein, an HPV16 papillomavirus tumor suppressor binding protein and an HPV18 papillomavirus tumor suppressor binding protein.

In another embodiment, the invention is a composition that includes at least one protein that includes at least a portion of at least one Toll-like Receptor agonist and at least a portion of at least one papillomavirus transforming protein, wherein the protein activates a Toll-like Receptor agonist.

A papillomavirus transforming protein (e.g., an E6 protein, such as at least a portion of SEQ ID NO: 136 (HPV16 E6); an E7 protein, such as at least a portion of SEQ ID NO: 151 (HPV16 E7)) can refer to a protein of the papillomavirus that participates in the conversion of a normal cell (e.g., an epithelial cell) into a neoplastic cell. The papillomavirus transforming protein can bind to at least one member selected from the group consisting of a pRB protein and a p107 protein.

The papillomavirus antigens for use in the compositions of the invention can include substitution of a cysteine residue in the native or naturally occurring amino acid sequence with another amino acid residue, such as alanine, glutamic acid, aspartic acid, glycine, isoleucine, leucine, glutamine, argenine, serine, or valine.

Exemplary cysteine residues suitable for substitution are shown in Table 3. It is believed that substitution of cysteine residues may reduce intradisulfide bonds in the papillomavirus antigens to thereby increase exposure of antigenic epitopes of the papillomavirus antigen.

TABLE 3

| Protein | SEQ ID NO: | Sequence (aa) positions of cyteinyl residues |
|---|---|---|
| HPV1 E6 | 137 | 16, 26, 29, 58, 59, 62, 99, 102, 132, 135 |
| HPV2 E6 | 139 | 13, 21, 35, 38, 68, 71, 88, 108, 111, 141, 144, |
| | | 148 |
| HPV5 E6 | 141 | 37, 41, 44, 54, 69, 73, 74, 77, 78, 114, 117, |
| | | 129, 130 |
| HPV6 E6 | 143 | 17, 31, 34, 64, 66, 67, 104, 107, 112, 131, 137, |
| | | 140, 144 |
| HPV8 E6 | 145 | 17, 21, 24, 34, 42, 53, 54, 56, 57, 58, 94, 97, |
| | | 109, 110 |
| HPV11 E6 | 147 | 17, 31, 34, 64, 66, 67, 104, 107, 112, 137, 140, |
| | | 144 |
| HPV16 E6 | 136 | 23, 37, 40, 58, 70, 73, 87, 104, 110, 113, 118, |
| | | 143, 146, 147 |
| HPV18 E6 | 149 | 18, 32, 35, 65, 68, 105, 108, 138, 141, 142 |
| HPV1 E7 | 152 | 26, 52, 55, 85 |
| HPV2 E7 | 154 | 26, 55, 56, 58, 68, 88, 91 |
| HPV5 E7 | 156 | 29, 56, 58, 61, 91, 94, 98 |
| HPV6 E7 | 158 | 25, 57, 58, 59, 61, 71, 91, 94 |
| HPV8 E7 | 160 | 29, 56, 58, 60, 61, 91, 94, 98 |
| HPV11 E7 | 161 | 25, 57, 58, 59, 61, 71, 91, 94 |
| HPV16 E7 | 151 | 24, 58, 59, 61, 68, 91, 94 |
| HPV18 E7 | 163 | 27, 63, 65, 66, 68, 98, 101 |
| HPV2 L2 | 169 | 10, 16 |
| HPV5 L2 | 171 | 19, 25 |
| HPV6 L2 | 173 | 21, 27 |
| HPV8 L2 | 175 | 19, 25 |
| HPV11 L2 | 176 | 20, 26 |
| HPV16 L2 | 166 | 22, 28 |
| HPV18 L2 | 178 | 21, 27 |
| HPV2 L1 | 180 | 101, 158, 172, 181, 221, 225, 319, 340, 373, |
| | | 422 |
| HPV5 L1 | 182 | 103, 161, 164, 175, 185, 229, 246, 255, 395, |
| | | 444 |
| HPV6 L1 | 184 | 99, 153, 157, 171, 181, 221, 225, 320, 341, |
| | | 374, 423 |
| HPV8 L1 | 186 | 103, 161, 164, 175, 184, 228, 245, 254, 394, |
| | | 443 |
| HPV11 L1 | 187 | 99, 172, 182, 222, 226, 321, 342, 375, 424, |
| | | 154, 158 |
| HPV16 L1 | 188 | 9, 20, 24, 38, 44, 88, 92, 187, 208, 242 |
| HPV18 L1 | 190 | 32, 153, 208, 212, 226, 236, 269, 276, 280, |
| | | 300, 340, 375, 396, 431, 480 |

In another embodiment, the invention is a composition that includes at least one protein that includes at least a portion of at least one Toll-like Receptor agonist and at least a portion of at least one papillomavirus capsid protein, wherein the protein activates a Toll-like Receptor.

In an additional embodiment, the invention is a composition that includes at least a portion of at least one Toll-like Receptor 5 agonist and at least a portion of at least one papillomavirus protein, wherein the papillomavirus protein includes at least one member selected from the group consisting of a papillomavirus E6 protein (e.g., SEQ ID NOS: 136, 137, 139, 141, 143, 145, 147, 149, 227), a papillomavirus E7 protein (e.g., SEQ ID NOS: 151, 152, 154, 156, 158, 160, 161, 163) and a papillomavirus L2 protein (e.g., SEQ ID NOS: 166, 167, 169, 171, 173, 175, 176, 178). The Toll-like Receptor 5 agonist and the papillomavirus protein can be components of a fusion protein.

In still another embodiment, the invention is a method of stimulating an immune response in a subject, comprising the step of administering to the subject a composition that includes at least one protein that includes at least a portion of at least one Toll-like Receptor agonist and at least a portion of at least one papillomavirus tumor suppressor binding protein, wherein the protein activates a Toll-like Receptor.

“At least a portion,” as used herein in reference to a flagellin (e.g., fljB/STF2, E. coli fliC, S. muenchen fliC), refers to any part of the flagellin (e.g., motif C; motif N; domain 1, 2, 3) or the entirety of the flagellin that can initiate an intracellular signal transduction pathway for a Toll-like Receptor.

In an embodiment, the flagellin component lacks at least a portion of a hinge region. The flagellin component can include at least one member selected from the group consisting of Salmonella typhimurium flagellin component, an E. coli flagellin component, a S. muenchen flagellin component, a Yersinia flagellin component, a Campylobacter flagellin component, a P. aeruginosa flagellin component and a L. monocytogenes flagellin component.

The compositions described herein (e.g., at least one papillomavirus protein component and at least one flagellin component) can further include at least one member selected from the group consisting of a Toll-like Receptor 2 agonist component, a Toll-like Receptor 3 agonist component, a Toll-like Receptor 4 agonist component, a Toll-like Receptor 6 agonist component, a Toll-like Receptor 7 agonist component, a Toll-like Receptor 8 agonist component, a Toll-like Receptor 9 agonist component, a Toll-like Receptor 10 agonist component, a Toll-like Receptor 11 agonist component and a Toll-like Receptor 12 agonist component.

At least one cysteine residue can be substituted for at least one amino acid residue in a naturally occurring flagellin amino acid sequence of the flagellin component.

The cysteine residue that substitutes for at least one amino acid residue in a naturally occurring flagellin amino acid sequence of the flagellin component can be remote to at least one amino acid of the Toll-like Receptor 5 recognition site of the flagellin component. “Toll-like Receptor 5 recognition site,” means that part of the TLR5 ligand (e.g., TLR5 agonist) that interacts with TLR5 to mediate a cellular response. “Toll-like Receptor 5 recognition site” is also referred to as a “Toll-like Receptor 5 activation site” and a “Toll-like Receptor 5 activation domain.”

Likewise, “Toll-like Receptor recognition site,” means that part of the Toll-like Receptor ligand (e.g., a Toll-like Receptor agonist) that interacts with its respective TLR to mediate a cellular response. “Toll-like Receptor recognition site” is also referred to as a “Toll-like Receptor activation site” and a “Toll-like Receptor activation domain.”

The HPV protein component can be chemically conjugated to flagellin components and Toll-like Receptor agonist components. Chemical conjugation (also referred to herein as “chemical coupling”) can include conjugation by a reactive group, such as a thiol group (e.g., a cysteine residue) or by derivatization of a primary (e.g., a amino-terminal) or secondary (e.g., lysine) group. Different crosslinkers can be used to chemically conjugate TLR ligands (e.g., TLR agonists) to HPV protein component. Exemplary cross linking agents are commerically available, for example, from Pierce (Rockland, Ill.). Methods to chemically conjugate the HPV component to the flagellin component are well-known and include the use of commercially available cross-linkers, such as those described herein.

For example, conjugation of an HPV protein component to a flagellin component or a Toll-like Receptor agonist component of the invention can be through at least one cysteine residue of the flagellin component or the Toll-like Receptor component and at least one cysteine residue of an HPV protein component employing established techniques. The HPV protein component can be derivatized with a homobifunctional, sulfhydryl-specific crosslinker; desalted to remove the unreacted crosslinker; and then the partner added and conjugated via at least one cysteine residue cysteine. Exemplary reagents for use in the conjugation methods can be purchased commercially from Pierce (Rockland, Ill.), for example, BMB (Catalog No: 22331), BMDB (Catalog No: 22332), BMH (Catalog No: 22330), BMOE (Catalog No: 22323), BM[PEO]₃ (Catalog No: 22336), BM[PEO]₄ (Catalog No:22337), DPDPB (Catalog No: 21702), DTME (Catalog No: 22335), HBVS (Catalog No: 22334).

Alternatively, the HPV protein component can also be conjugated to lysine residues on flagellin components, flagellin, Toll-like Receptor agonist components and Toll-like Receptor agonists of the invention. A protein component containing no cysteine residues is derivatized with a heterobifunctional amine and sulfhydryl-specific crosslinker. After desalting, the cysteine-containing partner is added and conjugated. Exemplary reagents for use in the conjugation methods can be purchased from Pierce (Rockland, Ill.), for example, AMAS (Catalog No: 22295), BMPA (Catalog No. 22296), BMPS (Catalog No: 22298), EMCA (Catalog No: 22306), EMCS (Catalog No: 22308), GMBS (Catalog No: 22309), KMUA (Catalog No: 22211), LC-SMCC (Catalog No: 22362), LC-SPDP (Catalog No: 21651), MBS (Catalog No: 22311), SATA (Catalog No: 26102), SATP (Catalog No: 26100), SBAP (Catalog No: 22339), SIA (Catalog No: 22349), SIAB (Catalog No: 22329), SMCC (Catalog No: 22360), SMPB (Catalog No: 22416), SMPH (Catalog No. 22363), SMPT (Catalog No: 21558), SPDP (Catalog No: 21857), Sulfo-EMCS (Catalog No: 22307), Sulfo-GMBS (Catalog No: 22324), Sulfo-KMUS (Catalog No: 21111), Sulfo-LC-SPDP (Catalog No: 21650), Sulfo-MBS (Catalog No: 22312), Sulfo-SIAB(Catalog No: 22327), Sulfo-SMCC (Catalog No: 22322), Sulfo-SMPB (Catalog No: 22317), Sulfo-LC-SMPT (Catalog No.: 21568).

Additionally, or alternatively, HPV protein components can also be conjugated to flagellin components or Toll-like Receptor agonist components of the invention via at least one lysine residue on both conjugate partners. The two conjugate partners are combined along with a homo-bifunctional amine-specific crosslinker. The appropriate hetero-conjugate is then purified away from unwanted aggregates and homo-conjugates. Exemplary reagents for use in the conjugation methods can be purchased from Pierce (Rockland, Ill.), for example, BSOCOES (Catalog No: 21600), BS₃ (Catalog No: 21580), DFDNB (Catalog No: 21525), DMA (Catalog No: 20663), DMP (Catalog No: 21666), DMS (Catalog No: 20700), DSG (Catalog No: 20593), DSP (Catalog No: 22585), DSS (Catalog No: 21555), DST (Catalog No: 20589), DTBP (Catalog No: 20665), DTSSP (Catalog No: 21578), EGS (Catalog No: 21565), MSA (Catalog No: 22605), Sulfo-DST (Catalog No: 20591), Sulfo-EGS (Catalog No: 21566), THPP (Catalog No: 22607).

Similarly, protein components can be conjugated to flagellin components or Toll-like Receptor agonist components of the invention via at least one carboxyl group (e.g., glutamic acid, aspartic acid, or the carboxy-terminus of the peptide or protein) on one partner and amines on the other partner. The two conjugation partners are mixed together along with the appropriate heterobifunctional crosslinking reagent. The appropriate hetero-conjugate is then purified away from unwanted aggregates and homo-conjugates. Exemplary reagents for use in the conjugation methods can be purchased from Pierce (Rockland, Ill.), for example, AEDP (Catalog No: 22101), EDC (Catalog No: 22980) and TFCS (Catalog No: 22299).

TLRs can be activated by nucleic acids. For example, TLR3 is activated by double-stranded (ds) RNA; TLR7 and TLR8 are activated by single-stranded (ss) RNA; and TLR9 is activated by CpG DNA sequences. Several different techniques can be employed to conjugate Toll-like Receptor agonist components of nucleic acid TLRs to HPV protein components. For example, for un-modified DNA molecule, the 5′ phosphate group can be modified with the water-soluble carbodiimide EDC (Pierce; Rockford, Ill., Catalog No: 22980) followed by imidazole to form a terminal phosphoylimidazolide. A terminal amine can then be substituted for this reactive group by the addition of ethylenediamine. The amine-modified nucleic acid can then be conjugated to a cysteine on an HPV protein component using a heterobifunctional maleimide (cysteine-specific) and NHS-ester (lysine-specific) crosslinker, as described above.

Alternatively, or additionally, a sulfhydryl group can be incorporated at the 5′ end by substituting cystamine for ethylenediamine in the second step. After reduction of the disulfide bond, the free sulfhydryl can be conjugated to cysteines on HPV protein components, flagellin components and, Toll-like Receptor agonist components employing a homo-bifunctional maleimide-based crosslinker, or to lysines using a heterobifunctional crosslinker, as described above. Alternately, or additionally, synthetic oligonucleotides can be synthesized with modified bases on either the 5′ or 3′ end. These modified bases can include primary amine or sulfhydryl groups which can be conjugated to proteins using the methods described above. The 3′ end of RNA molecules may be chemically modified to allow coupling with proteins or other macromolecules. The diol on the 3′-ribose residue may be oxidized using sodium meta-periodate to produce an aldehyde group. The aldehyde may then be conjugated to proteins using a hydrazide-containing crosslinker, such as MPBH (Pierce; Rockland, Ill., Catalog No: 22305), which covalently modifies the carbonyl group, and then conjugates to free thiols on proteins via a maleimide group. Alternatively, the 3′ hydroxyl group may be derivatized directly with an isocyanate-containing crosslinker, such as PMPI (Pierce; Rockland, Ill., Catalog No: 28100), which also contains a maleimide group for conjugation to protein sulfhydryls.

Synthetic small-molecule TLR ligands (e.g., Toll-like Receptor agonists, Toll-like Receptor antagonists) have been identified. For example, imiquimod, which potently activates TLR7, and resiquimod, an activator of both TLR7 and TLR8. Analogs of these compounds with varying levels of potency and specificity have been synthesized. The ability to conjugate a small molecule TLR agonist to an HPV protein component can depend on the chemical nature of the TLR ligand. Some TLR agonists may have active groups that can be exploited for chemical conjugation. For example, imiquimod, as well as its analog gardiquimod (InvivoGen; San Diego, Calif.) and the TLR7-activating adenine analog CL087 (InvivoGen, San Diego, Calif.), have primary amine groups (—NH₂), which can be targets for derivatization by crosslinkers containing imidoesters or NHS esters. In another strategy, gardiquimod contains an exposed hydroxyl (—OH) group, which can be derivatized by an isocyanate-containing crosslinker, such as PMPI (Pierce; Rockland, Ill. Catalog No: 28100) for subsequent crosslinking to protein sulfhydryl groups.

In addition, custom synthesis of derivatives of small-molecule TLR agonists can be arranged in order to attach novel functional groups at different positions in the molecule to facilitate crosslinking. Custom derivatives can also include groups, such as maleimides, which can then be used for direct linking to protein antigens.

Chemical conjugation of an HPV protein component to the flagellin component can result in increased aqueous solubility of the antigen (e.g., an essentially hydrophobic antigen, such as a maturational cleavage site antigen) as a component of the composition.

The composition comprising a flagellin component that is at least a portion of a flagellin, wherein the flagellin component includes at least one cysteine residue and whereby the flagellin component activates a Toll-like Receptor 5 can include a cysteine residue in the hypervariable region of the flagellin component.

At least one cysteine residue substitutes for at least one amino acid in a naturally occurring flagellin amino acid sequence flagellin component. The cysteine residue can substitute for at least one amino acid selected from the group consisting of amino acid 1, 237, 238, 239, 240, 241 and 495 of SEQ ID NO: 99; at least one amino acid selected from the group consisting of amino acid 1, 240, 241, 242, 243, 244 and 505 of SEQ ID NO: 98; at least one amino acid selected from the group consisting of amino acid 1, 237, 238, 239, 240, 241 and 504 of SEQ ID NO: 102; at least one amino acid selected from the group consisting of amino acid 1, 211, 212, 213 and 393 of SEQ ID NO: 104; at least one amino acid selected from the group consisting of amino acid 1, 151, 152, 153, 154 and 287 of SEQ ID NO: 105; at least one amino acid selected from the group consisting of amino acid 1, 238, 239, 240, 241, 242, 243 and 497 of SEQ ID NO: 106; at least one amino acid selected from the group consisting of amino acid 1, 237, 238, 239, 240, 241 and 495 of SEQ ID NO: 99.

The flagellin component or Toll-like Receptor agonist component can include at least a portion of a naturally occurring flagellin amino acid sequence in combination with the cysteine residue.

The composition of the invention wherein the cysteine residue substitutes for at least one amino acid in a naturally occurring flagellin amino acid sequence flagellin component, or wherein the flagellin component includes at least a portion of a naturally occurring flagellin amino acid sequence in combination with the cysteine residue, can activate a Toll-like Receptor 5. For example, a cysteine residue can be placed within the D1/D2 domain proximate to the amino-terminus and carboxy-terminus, remote to the TLR5 recognition site (see, for example, FIGS. 4 and 5). Alternatively, or additionally, the cysteine residue can be placed at the distal point of the hypervariable domain (see, for example, FIGS. 4 and 5) at about amino acid 237, about 238, about 239, about 240 and about 241 of SEQ ID NO: 99. Substituting polar or charged amino acids is preferable to substituting hydrophobic amino acids with cysteine residues. Substitution within the TLR5 recognition site is least preferable.

Flagellin from Salmonella typhimurium STF1 (F1iC) is depicted in SEQ ID NO: 99 (Accession No: P06179). The TLR5 recognition site is amino acid about 79 to about 117 and about 408 to about 439 of SEQ ID NO: 99. Cysteine residues can substitute for or be included in combination with amino acid about 408 to about 439 of SEQ ID NO: 99; amino acids about 1 and about 495 of SEQ ID NO: 99; amino acids about 237 to about 241 of SEQ ID NO: 99; and/or amino acids about 79 to about 117 and about 408 to about 439 of SEQ ID NO: 99.

Salmonella typhimurium flagellin STF2 (F1jB) is depicted in SEQ ID NO: 98. The TLR5 recognition site is amino acids about 80 to about 118 and about 420 to about 451 of SEQ ID NO: 98. Cysteine residues can substitute for or be included in combination with amino acids about 1 and about 505 of SEQ ID NO: 98; amino acids about 240 to about 244 of SEQ ID NO: 98; amino acids about 79 to about 117 and/or about 419 to about 450 of SEQ ID NO: 98.

Salmonella muenchen flagellin is depicted in SEQ ID NO: 102 (Accession No: #P06179). The TLR5 recognition site is amino acids about 79 to about 117 and about 418 to about 449 of SEQ ID NO: 102. Cysteine residues can substitute for or be included in combination with amino acids about 1 and about 504 of SEQ ID NO: 102; about 237 to about 241 of SEQ ID NO: 102; about 79 to about 117; and/or about 418 to about 449 of SEQ ID NO: 102.

Escherichia coli flagellin is depicted in SEQ ID NO: 106 (Accession No: P04949). The TLR5 recognition site is amino acids about 79 to about 117 and about 410 to about 441 of SEQ ID NO: 106. Cysteine residues can substitute for or be included in combination with amino acids about 1 and about 497 of SEQ ID NO: 106; about 238 to about 243 of SEQ ID NO: 106; about 79 to about 117; and/or about 410 to about 441 of SEQ ID NO: 106.

Pseudomonas auruginosa flagellin is depicted in SEQ ID NO: 104. The TLR5 recognition site is amino acids about 79 to about 114 and about 308 to about 338 of SEQ ID NO: 104. Cysteine residues can substitute for or be included in combination with amino acids about 1 and about 393 of SEQ ID NO: 104; about 211 to about 213 of SEQ ID NO: 104; about 79 to about 114; and/or about 308 to about 338 of SEQ ID NO: 104.

Listeria monocytogenes flagellin is depicted in SEQ ID NO: 105. The TLR5 recognition site is amino acids about 78 to about 116 and about 200 to about 231 of SEQ ID NO: 105. Cysteine residues can substitute for or be included in combination with amino acids about 1 and about 287 of SEQ ID NO: 105; about 151 to about 154 of SEQ ID NO: 105; about 78 to about 116; and/or about 200 to about 231 of SEQ ID NO: 105.

Experimentally defined TLR5 recognition sites on STF2 have been described (see, for example, Smith, K. D., et al., Nature Immunology 4:1247-1253 (2003) at amino acids about 79 to about 117 and about 420 to about 451. In addition, Smith, K. D., et al., Nature Immunology 4:1247-1253 (2003), based on sequence homology, identified TLR5 recognition sites on other flagellins, such as STF1 at amino acids about 79 to about 117, about 408 to about 439; P. aeruginosa at amino acids about 79 to about 117, about 308 to about 339; L. pneumophila at amino acids about 79 to about 117, about 381 to about 419; E. coli at amino acids about 79 to about 117, about 477, about 508; S. marcesens at amino acids about 79 to about 117, about 265-about 296; B. subtilus at amino acids about 77 to about 117, about 218 to about 249; and L. monocytogenes at amino acids about 77 to about 115, about 200 to about 231.

The high-resolution structure STF1 (F1iC) (SEQ ID NO: 99) has been determined and can be a basis for analysis of TLR5 recognition by a flagellin and the location of cysteine substitutions/additions. Flagellin resembles a “boomerang,” with the amino- and carboxy-termini at the end of one arm (see, for example, FIGS. 4 and 5). The TLR5 recognition site is located roughly on the outer side of the boomerang just below the bend toward the amino- and carboxy-termini of the flagellin. The hinge region, which is not required for TLR5 recognition, is located above the bend. The region of greatest sequence homology of flagellins is in the TLR5 recognition site. The next region of sequence homology is in the D1 and D2 domains, which include the TLR5 recognition site and the amino- and carboxy-termini. The D0 and D1 domains, with or without a linker, is STF2Δ (SEQ ID NO: 101), which can activate TLR5. The region of least sequence homology between flagellins is the hypervariable region.

It is believed that the ability of the flagellin component or Toll-like Receptor agonist component to activate TLR5 can be accomplished by maintaining the conjugation sites (cysteine residues substituted for at least one amino acid in a naturally occurring flagellin amino acid sequence flagellin component or at least a portion of a naturally occurring flagellin amino acid sequence in combination with the cysteine residue) remote from the TLR5 or TLR recognition site. For example, for STF1 (SEQ ID NO: 99), for which a high resolution structural determination is available, this may be achieved in the D1 domain, D2 domain or in the hinge region. In the D1/D2 domain the amino- and carboxy-termini can be remote (also referred to herein as “distal”) from the TLR5 recognition site, and moving away from either the amino or carboxy terminus may bring the conjugation site closer to the recognition site and may interfere with TLR5 activity. In the hinge region amino acids about 237 to about 241 of SEQ ID NO: 99, are approximately at the other tip of the “boomerang” and are about the same distance from the TLR5 recognition site as the amino- and carboxy-termini. This site may also be a location that maintains TLR5 recognition.

Amino acid identity can be taken into consideration for the location of conjugation sites. Polar and charged amino acids (e.g., serine, aspartic acid, lysine) are more likely to be surface exposed and amenable to attachment of an antigen. Hydrophobic amino acids (e.g., valine, phenalalanine) are more likely to be buried and participate in structural interactions and should be avoided.

Compositions that include flagellin components with cysteine residues or Toll-like Receptor agonist components with cysteine residues activate TLR5 and can be chemically conjugated to antigens.

The composition comprising a flagellin component that is at least a portion of a flagellin, wherein the flagellin component includes at least one cysteine residue and whereby the flagellin component activates a Toll-like Receptor 5 can include at least one lysine of the flagellin component that has been substituted with at least one member selected from the group consisting of an arginine residue, a serine residue and a histidine residue.

In an additional embodiment, the Toll-like Receptor agonist component include at least a portion of a Toll-like Receptor agonist, wherein the Toll-like Receptor agonist component includes at least one cysteine residue in a position where a cysteine residue does not occur in the native Toll-like Receptor agonist, whereby the Toll-like Receptor agonist component activates a Toll-like Receptor.

The cysteine residue in the composition comprising a Toll-like Receptor agonist component that is at least a portion of a Toll-like Receptor agonist, wherein the Toll-like Receptor agonist component includes at least one cysteine residue in a position where a cysteine residue does not occur in the native Toll-like Receptor agonist, whereby the Toll-like Receptor agonist component activates a Toll-like Receptor, substitutes for at least one amino acid in a naturally occurring amino acid sequence of a Toll-like Receptor agonist component. The cysteine can substitute for at least one amino acid remote to the Toll-like Receptor recognition site of the Toll-like Receptor agonist component.

The Toll-like Receptor agonist component includes at least a portion of a naturally occurring Toll-like Receptor agonist amino acid sequence in combination with a cysteine residue. The cysteine residue in combination with the naturally occurring Toll-like Receptor agonist can be remote to the Toll-like Receptor recognition site of the Toll-like Receptor agonist component.

The composition can include at least a portion of a papillomavirus protein and at least a portion of a Toll-like Receptor agonist component that is at least a portion of a Toll-like Receptor agonist, wherein the Toll-like Receptor agonist component includes at least one cysteine residue in a position where a cysteine residue does not occur in the native Toll-like Receptor agonist, whereby the Toll-like Receptor agonist component activates a Toll-like Receptor can further include at least a portion of at least one HPV protein component.

The composition can include at least a portion of a papillomavirus protein and a flagellin component that is at least a portion of a flagellin, wherein at least one lysine of the flagellin component has been substituted with at least one arginine, whereby the flagellin component activates a Toll-like Receptor 5.

“Substituted,” as used herein in reference to the flagellin, flagellin component, Toll-like Receptor agonist or Toll-like Receptor agonist component, means that at least one amino acid, such as a lysine of the flagellin component, has been modified to another amino acid residue, for example, a conservative substitution (e.g., arginine, serine, histidine) to thereby form a substituted flagellin component or substituted Toll-like Receptor agonist component. The substituted flagellin component or substituted Toll-like Receptor agonist component can be made by generating recombinant constructs that encode flagellin with the substitutions, by chemical means, by the generation of proteins or peptides of at least a portion of the flagellin by protein synthesis techniques, or any combination thereof.

The lysine residue that is substituted with an amino acid (e.g., arginine, serine, histidine) can be at least one lysine residue selected from the group consisting of lysine 19, 41, 58, 135, 160, 177, 179, 203, 215, 221, 228, 232, 241, 251, 279, 292, 308, 317, 326, 338, 348, 357, 362, 369, 378, 384, 391 and 410 of SEQ ID NO: 99.

The flagellin can be a S. typhimurium flagellin that includes SEQ ID NO: 100. The lysine residue that is substituted with an amino acid (e.g., arginine, serine, histidine) can be at least one lysine residue selected from the group consisting of lysine 20, 42, 59, 136, 161, 177, 182, 189, 209, 227, 234, 249, 271, 281, 288, 299, 319, 325, 328, 337, 341, 355, 357, 369, 381, 390, 396, 403, 414 and 422 of SEQ ID NO: 100.

The flagellin can be an E. coli fliC that includes SEQ ID NO: 107. The flagellin can be a S. muenchen that include the includes SEQ ID NO: 103. The flagellin can be a P. aeruginosa flagellin that includes SEQ ID NO: 104. The flagellin can be a Listeria monocytogenes flagellin that includes SEQ ID NO: 105.

The compositions of the invention can include a flagellin that has lysines substituted in a region adjacent to the motif C of the flagellin, the motif N of the flagellin, both the motif C and the motif N of the flagellin, domain 1 of the flagellin, domain 2 of the flagellin or any combination thereof. Motif C and motif N of flagellin and domain 1 and domain 2 of the flagellin can be involved in activation of TLR5 by the flagellin (Murthy, et al., J. Biol. Chem., 279:5667-5675 (2004)).

Certain lysine residues in flagellin are near or in domain 1, the motif C or motif N, motifs can be important in binding of the flagellin to TLR5. For example, lysine residues at amino acids 58, 135, 160 and 410 of SEQ ID NO: 99 may be substituted with at least one member selected from the group consisting of an arginine residue, a serine residue and a histidine residue. Derivatization of such lysine residues to, for example, chemically conjugated antigens to flagellins, may decrease the ability or the binding affinity of the flagellin to TLR5 and, thus, diminish an innate immune response mediated by TLR5. Substitution of at least one lysine residue in a flagellin that may be near to regions of the flagellin that are important in mediating interactions with TLR5 (e.g., motif C, motif N, domain 1) with another amino acid (e.g., arginine, serine, histidine) may preserve or enhance flagellin binding to TLR5. In a particular embodiment, the amino acid substitution is a conservative amino acid substitution with at least one member selected from the group consisting of arginine, serine and histidine. Exemplary commercially available reagents for chemical conjugation are described herein.

Certain lysine residues in flagellin are in the domain (domain 1) and can be important for activation of TLR5. For example, lysine residues at positions 58, 135, 160 and 410 of SEQ ID NO: 99 are in domain 1. Derivatization of such lysine residues to, for example, chemically conjugated antigens, may decrease TLR5 bioactivity and, thus, diminish an innate immune response mediated by TLR5.

Lysine residues that can be substituted can include lysine residues implicated in TLR5 activation. Lysine residues in motif N (amino acids 95-108 of SEQ ID NO: 99) and/or motif C (amino acids 441-449 of SEQ ID NO: 99) can be suitable for substitution. Substitution of certain lysine residue in the flagellin (e.g., lysine at amino acid position 19, 41) with, for example, an arginine, serine or histidine, can maintain binding of the flagellin to TLR5 and leave other lysines available for chemical conjugate to another molecule, such as an antigen (e.g., protein) or another molecule, such as another protein, peptide or polypeptide.

The X-ray crystal structure of the F41 fragment of flagellin from Salmonella typhimurium shows the domain structure of flagellin (Samatey, F. A., et al., Nature 410:321 (2001)). The full length flagellin protein contains 4 domains, designated as D0, D1, D2 and D3. Three of these domains are shown in the crystal structure because the structure was made with a proteolytic fragment of full length flagellin. The amino acid sequences of Salmonella typhimurium flagellin for these regions, numbered relative to SEQ ID NO: 99 are as follows:
D0 contains the regions A1 through A55 and S451 through R494

D1 contains the regions N56 through Q176 and T402 through R450

D2 contains the regions K177 through G189 and A284 through A401

D3 contains the region Y190 though V283

Exemplary lysine residues of SEQ ID NO: 812 suitable for substitution with, for example, arginine, histidine, or serine, can include:

D0 contains 2 lysine residues; K19, K41

D1 contains 4 lysine residues; K58, K135, K160 and K410

D2 contains 14 lysine residues at positions 177, 179, 292, 308, 317, 326, 338, 348, 357, 362, 369, 378, 384, 391

D3 contains 8 lysine residues at positions 203, 215, 221, 228, 232, 241, 251, 279

Exemplary lysine residues suitable for substitution include lysines at positions 58, 135, 160 and 410 of SEQ ID NO: 99 (Jacchieri, S. G., et. al., J. Bacteriol. 185:4243 (2003); Donnelly, M. A., et al., J. Biol. Chem. 277:40456 (2002)). The sequences were obtained from the Swiss-Prot Protein Knowledgebase located online at http://us.expasy.org/sprot/. Lysine residues that can be modified are indicated with a *.

Exemplary lysine residues of SEQ ID NO: 100 suitable for substitution can include:

D0—with two lysines at positions 20, 42;

D1—with five lysines at positions 59, 136, 161, 414, 422;

D2—with sixteen lysines at positions 177, 182, 189, 299, 319, 325, 328, 337, 341, 355, 357, 369, 381, 390, 396, 403 and

D3—with seven lysines at positions 209, 227, 234, 249, 271, 281, 288.

In an additional embodiment, the invention includes a protein, peptide polypeptide having at least about 70%, at least about 75%, at least about 80%, at least about 85%, at least about 90%, at least about 95%, at least about 98% and at least about 99% sequence identity to the proteins, HPV protein components and flagellin components of the invention.

The percent identity of two amino acid sequences (or two nucleic acid sequences) can be determined by aligning the sequences for optimal comparison purposes (e.g., gaps can be introduced in the sequence of a first sequence). The amino acid sequence or nucleic acid sequences at corresponding positions are then compared, and the percent identity between the two sequences is a function of the number of identical positions shared by the sequences (i.e., % identity=# of identical positions/total # of positions×100). The length of the protein or nucleic acid encoding can be aligned for comparison purposes is at least 30%, preferably, at least 40%, more preferably, at least 60%, and even more preferably, at least 70%, 75%, 80%, 85%, 90%, 95%, 98%, 99% or 100%, of the length of the reference sequence, for example, the nucleic acid sequence of a papillomavirus protein (e.g., SEQ ID NOS: 137, 139, 141, 143, 145, 147, 149, 152, 154, 156, 158, 160, 161, 163, 167, 169, 171, 173, 175, 176, 178, 180, 182, 184, 186, 188, 190, 216-222), Toll-like Receptor agonist (e.g., SEQ ID NOs: 135, 193, 194) or fusion protein (e.g., SEQ ID NOs: 208, 209, 210, 211, 212, 213, 214, 215) of the invention.

The actual comparison of the two sequences can be accomplished by well-known methods, for example, using a mathematical algorithm. A preferred, non-limiting example of such a mathematical algorithm is described in Karlin et al. (Proc. Natl. Acad. Sci. USA, 90:5873-5877 (1993), the teachings of which are hereby incorporated by reference in its entirety). Such an algorithm is incorporated into the BLASTN and BLASTX programs (version 2.2) as described in Schaffer et al. (Nucleic Acids Res., 29:2994-3005 (2001), the teachings of which are hereby incorporated by reference in its entirety). When utilizing BLAST and Gapped BLAST programs, the default parameters of the respective programs (e.g., BLASTN; available at the Internet site for the National Center for Biotechnology Information) can be used. In one embodiment, the database searched is a non-redundant (NR) database, and parameters for sequence comparison can be set at: no filters; Expect value of 10; Word Size of 3; the Matrix is BLOSUM62; and Gap Costs have an Existence of 11 and an Extension of 1.

Another mathematical algorithm employed for the comparison of sequences is the algorithm of Myers and Miller, CABIOS (1989), the teachings of which are hereby incorporated by reference in its entirety. Such an algorithm is incorporated into the ALIGN program (version 2.0), which is part of the GCG (Accelrys, San Diego, Calif.) sequence alignment software package. When utilizing the ALIGN program for comparing amino acid sequences, a PAM120 weight residue table, a gap length penalty of 12, and a gap penalty of 4 is used. Additional algorithms for sequence analysis are known in the art and include ADVANCE and ADAM as described in Torellis and Robotti (Comput. Appl. Biosci., 10: 3-5 (1994), the teachings of which are hereby incorporated by reference in its entirety); and FASTA described in Pearson and Lipman (Proc. Natl. Acad. Sci. USA, 85: 2444-2448 (1988), the teachings of which are hereby incorporated by reference in its entirety).

The percent identity between two amino acid sequences can also be accomplished using the GAP program in the GCG software package (Accelrys, San Diego, Calif.) using either a Blossom 63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the GCG software package (Accelrys, San Diego, Calif.), using a gap weight of 50 and a length weight of 3.

The nucleic acid sequence encoding a HPV protein component, or flagellin component of the invention and polypeptides of the invention can include nucleic acid sequences that hybridize to nucleic acid sequences or complements of nucleic acid sequences of the invention and nucleic acid sequences that encode amino acid sequences and fusion proteins of the invention under selective hybridization conditions (e.g., highly stringent hybridization conditions). As used herein, the terms “hybridizes under low stringency,” “hybridizes under medium stringency,” “hybridizes under high stringency,” or “hybridizes under very high stringency conditions,” describe conditions for hybridization and washing of the nucleic acid sequences. Guidance for performing hybridization reactions, which can include aqueous and nonaqueous methods, can be found in Aubusel, F. M., et al., Current Protocols in Molecular Biology, John Wiley & Sons, N.Y. (2001), the teachings of which are hereby incorporated herein in its entirety.

For applications that require high selectivity, relatively high stringency conditions to form hybrids can be employed. In solutions used for some membrane based hybridizations, addition of an organic solvent, such as formamide, allows the reaction to occur at a lower temperature. High stringency conditions are, for example, relatively low salt and/or high temperature conditions. High stringency are provided by about 0.02 M to about 0.10 M NaCl at temperatures of about 50° C. to about 70° C. High stringency conditions allow for limited numbers of mismatches between the two sequences. In order to achieve less stringent conditions, the salt concentration may be increased and/or the temperature may be decreased. Medium stringency conditions are achieved at a salt concentration of about 0.1 to 0.25 M NaCl and a temperature of about 37° C. to about 55° C., while low stringency conditions are achieved at a salt concentration of about 0.15 M to about 0.9 M NaCl, and a temperature ranging from about 20° C. to about 55° C. Selection of components and conditions for hybridization are well known to those skilled in the art and are reviewed in Ausubel et al. (1997, Short Protocols in Molecular Biology, John Wiley & Sons, New York N.Y., Units 2.8-2.11, 3.18-3.19 and 4-64.9).

A further embodiment of the invention is a method of stimulating an immune response in a subject, comprising the step of administering to the subject a composition that includes at least a portion of at least one Toll-like Receptor agonist and at least a portion of at least one papillomavirus transforming protein, wherein the protein activates a Toll-like Receptor agonist.

Another embodiment of the invention is a method of stimulating an immune response in a subject, comprising the step of administering to the subject a composition that includes at least a portion of at least one Toll-like Receptor agonist and at least a portion of at least one papillomavirus capsid protein, wherein the protein activates a Toll-like Receptor.

In yet another embodiment, the invention is a method of stimulating an immune response in a subject, comprising the step of administering to the subject a composition that includes a papillomavirus protein, wherein the papillomavirus protein includes at least a portion of at least a portion of a Toll-like Receptor 5 agonist and at least a portion of at least one member selected from the group consisting of a papillomavirus E6 protein, a papillomavirus E7 protein and a papillomavirus L2 protein.

The compositions of the invention can be employed to stimulate a protective immune response in a subject.

“Stimulating an immune response,” as used herein, refers to the generation of antibodies and/or T-cells to at least a portion of the protein, the HPV protein component of the proteins described herein. The antibodies and/or T-cells can be generated to at least a portion of papillomavirus protein (e.g., E6, E7, L2 protein).

Stimulating an immune response in a subject can include the production of humoral and/or cellular immune responses that are reactive against the antigen, such as a papillomavirus protein.

The compositions of the invention for use in methods to stimulate immune responses in subjects, can be evaluated for the ability to stimulate an immune response in a subject using well-established methods. Exemplary methods to determine whether the compositions of the invention stimulate an immune response in a subject, include measuring the production of antibodies specific to the antigen (e.g., IgG antibodies) by a suitable technique such as, ELISA assays; the potential to induce antibody-dependent enhancement (ADE) of a secondary infection; macrophage-like assays; and the ability to generate serum antibodies in non-human models (e.g., mice, rabbits, monkeys) (Putnak, et al., Vaccine 23:4442-4452 (2005)).

“Stimulates a protective immune response,” as used herein, means administration of the compositions of the invention results in production of antibodies to the protein to thereby cause a subject to survive challenge by an otherwise lethal dose of a papillomavirus protein. Techniques to determine a lethal dose of a virus are known to one of skill in the art. Exemplary techniques for determining a lethal dose can include administration of varying doses of virus and a determination of the percent of subjects that survive following administration of the dose of virus (e.g., LD₁₀, LD₂₀, LD₄₀, LD₅₀, LD₆₀, LD₇₀, LD₈₀, LD₉₀). For example, a lethal dose of a virus that results in the death of 50% of a population of subjects is referred to as an “LD₅₀”; a lethal dose of a virus that results in the death of 80% of a population of subjects is referred to herein as “LD₈₀”; a lethal dose of a virus that results in death of 90% of a population of subjects is referred to herein as “LD₉₀.”

For example, determination of the LD₉₀ can be conducted in subjects (e.g., mice) by implanting subcutaneously varying doses (e.g., dilutions, such as log and half-log dilutions) of mouse TC-1 lung tumor cells and measuring tumor volume for about 5 to about 6 weeks post-implantation. Protective immunity in experimentally-immunized mice compared to non-immunized mice can be assessed, for example, by measuring a reduction in tumor volume, a delay in onset of tumor growth, or an increase in survival time of the experimentally-immunized host compared to the non-immunized host (Bermúdez-Humarán, Luis G, et al., J. Immunol., 175: 7297-7302 (2005); Qian, X, et al., Immunol Lett., 102:191-201 (2006)). Fusion proteins described herein can be made in a prokaryotic host cell or a eukaryotic host cell. The prokaryotic host cell can be at least one member selected from the group consisting of an E. coli prokaryotic host cell, a Pseudomonas prokaryotic host cell, a Bacillus prokaryotic host cell, a Salmonella prokaryotic host cell and a P. fluorescens prokaryotic host cell. The eukaryotic host cell can include a Saccharomyces eukaryotic host cell, an insect eukaryotic host cell (e.g., at least one member selected from the group consisting of a Baculovirus infected insect cell, such as Spodoptera frugiperda (Sf9) or Trichoplusia ni (High5) cells; and a Drosophila insect cell, such as Dme12 cells), a fungal eukaryotic host cell, a parasite eukaryotic host cell (e.g., a Leishmania tarentolae eukaryotic host cell), CHO cells, yeast cells (e.g., Pichia) and a Kluyveromyces lactis host cell.

Suitable eukaryotic host cells to make the fusion proteins described herein and vectors can also include plant cells (e.g., tomato; chloroplast; mono- and dicotyledonous plant cells; Arabidopsis thaliana; Hordeum vulgare; Zea mays; potato, such as Solanum tuberosum; carrot, such as Daucus carota L.; and tobacco, such as Nicotiana tabacum, Nicotiana benthamiana (Gils, M., et al., Plant Biotechnol J. 3:613-20 (2005); He, D. M., et al., Colloids Surf B Biointerfaces, (2006); Huang, Z., et al., Vaccine 19:2163-71 (2001); Khandelwal, A., et al., Virology. 308:207-15 (2003); Marquet-Blouin, E., et al., Plant Mol Biol 51:459-69 (2003); Sudarshana, M. R., et al. Plant Biotechnol J. 4:551-9 (2006); Varsani, A., et al., Virus Res, 120:91-6 (2006); Kamarajugadda S., et al., Expert Rev Vaccines 5:839-49 (2006); Koya V, et al., Infect Immun. 73:8266-74 (2005); Zhang, X., et al., Plant Biotechnol J. 4:419-32 (2006)).

The fusion proteins of the invention can be made by well-established methods and can be purified and characterized employing well-known methods (e.g., gel chromatography, cation exchange chromatography, SDS-PAGE), as described herein.

In an embodiment, the methods of making a protein of the invention, in particular, a fusion protein, can include a step of deleting a signal sequence of the fusion protein or component of the fusion protein (e.g., an HPV antigen) in the nucleic acid sequence encoding the fusion protein or component of the fusion protein to thereby prevent secretion of the protein in the host cell, which results in accumulation of the protein in the cell. The accumulated protein can be purified from the cell.

In another embodiment, the methods of making a protein of the invention, in particular, a fusion protein, can include a step of deleting at least one glycosylation site (e.g., an N-glycosylation site NXST (SEQ ID NO: 226)) in the nucleic acid sequence encoding the fusion protein or component of the fusion protein (e.g., at least a portion of an HPV antigen, at least a portion of a flagellin).

A “subject,” as used herein, can be a mammal, such as a primate or rodent (e.g., rat, mouse). In a particular embodiment, the subject is a human.

An “effective amount,” when referring to the amount of a composition and fusion protein of the invention, refers to that amount or dose of the composition and fusion protein, that, when administered to the subject is an amount sufficient for therapeutic efficacy (e.g., an amount sufficient to stimulate an immune response in the subject). The compositions and fusion proteins of the invention can be administered in a single dose or in multiple doses.

The methods of the present invention can be accomplished by the administration of the compositions and fusion proteins of the invention by enteral or parenteral means. Specifically, the route of administration is by oral ingestion (e.g., drink, tablet, capsule form) or intramuscular injection of the composition and fusion protein. Other routes of administration as also encompassed by the present invention including intravenous, intradermal, intraarterial, intraperitoneal, or subcutaneous routes, and nasal administration. Suppositories or transdermal patches can also be employed.

The compositions and proteins of the invention can be administered ex vivo to a subject's autologous dendritic cells. Following exposure of the dendritic cells to the composition and protein of the invention, the dendritic cells can be administered to the subject.

The compositions and proteins of the invention can be administered alone or can be coadministered to the patient. Coadministration is meant to include simultaneous or sequential administration of the composition, protein or polypeptide of the invention individually or in combination. Where the composition and protein are administered individually, the mode of administration can be conducted sufficiently close in time to each other (for example, administration of the composition close in time to administration of the fusion protein) so that the effects on stimulating an immune response in a subject are maximal. It is also envisioned that multiple routes of administration (e.g., intramuscular, oral, transdermal) can be used to administer the compositions and proteins of the invention.

The compositions and proteins of the invention can be administered alone or as admixtures with conventional excipients, for example, pharmaceutically, or physiologically, acceptable organic, or inorganic carrier substances suitable for enteral or parenteral application which do not deleteriously react with the extract. Suitable pharmaceutically acceptable carriers include water, salt solutions (such as Ringer's solution), alcohols, oils, gelatins and carbohydrates such as lactose, amylose or starch, fatty acid esters, hydroxymethycellulose, and polyvinyl pyrrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like which do not deleteriously react with the compositions, proteins or polypeptides of the invention. The preparations can also be combined, when desired, with other active substances to reduce metabolic degradation. The compositions and proteins of the invention can be administered by is oral administration, such as a drink, intramuscular or intraperitoneal injection or intranasal delivery. The compositions and proteins alone, or when combined with an admixture, can be administered in a single or in more than one dose over a period of time to confer the desired effect (e.g., alleviate or prevent HPV infection, to alleviate symptoms of HPV infection).

When parenteral application is needed or desired, particularly suitable admixtures for the compositions and proteins are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. The compositions, proteins or polypeptides can also be incorporated into liposomes or administered via transdermal pumps or patches. Pharmaceutical admixtures suitable for use in the present invention are well-known to those of skill in the art and are described, for example, in Pharmaceutical Sciences (17th Ed., Mack Pub. Co., Easton, Pa.) and WO 96/05309 the teachings of which are hereby incorporated by reference.

The compositions and proteins of the invention can be administered to a subject on a support that presents the compositions, proteins and polypeptides of the invention to the immune system of the subject to generate an immune response in the subject. The presentation of the compositions, proteins and polypeptides of the invention would preferably include exposure of antigenic portions of the viral protein to generate antibodies. The components (e.g., PAMP and a viral protein) of the compositions, proteins and polypeptides of the invention are in close physical proximity to one another on the support. The compositions and proteins of the invention can be attached to the support by covalent or noncovalent attachment. Preferably, the support is biocompatible. “Biocompatible,” as used herein, means that the support does not generate an immune response in the subject (e.g., the production of antibodies). The support can be a biodegradable substrate carrier, such as a polymer bead or a liposome. The support can further include alum or other suitable adjuvants. The support can be a virus (e.g., adenovirus, poxvirus, alphavirus), bacteria (e.g., Salmonella) or a nucleic acid (e.g., plasmid DNA).

The dosage and frequency (single or multiple doses) administered to a subject can vary depending upon a variety of factors, including prior exposure to an HPV antigen, an HPV protein, the duration of HPV infection, prior treatment of the HPV infection, the route of administration of the composition, protein or polypeptide; size, age, sex, health, body weight, body mass index, and diet of the subject; nature and extent of symptoms of viral exposure, viral infection and the particular viral responsible for the HPV infection or treatment or infection of an HPV antigen, kind of concurrent treatment, complications from the viral exposure, viral infection or exposure or other health-related problems. Other therapeutic regimens or agents can be used in conjunction with the methods and compositions, proteins or polypeptides of the present invention. For example, the administration of the compositions and proteins can be accompanied by other viral therapeutics or use of agents to treat the symptoms of a condition associated with or consequent to exposure to the HPV, and HPV antigen, or HPV infection, for example. Adjustment and manipulation of established dosages (e.g., frequency and duration) are well within the ability of those skilled in the art.

The composition and/or dose of the fusion proteins can be administered to the human in a single dose or in multiple doses, such as at least two doses. When multiple doses are administered to the subject, a second or dose in addition to the initial dose can be administered days (e.g., 1, 2, 3, 4, 5, 6 or 7), weeks (e.g., 1, 2, 3, 4, 5, 6, 7, 8, 9, 10), months (e.g., 1, 2, 3, 4, 5, 6, 7, 8, 9, 10) or years (e.g., 1, 2, 3, 4, 5, 6, 7, 8, 9, 10) after the initial dose. For example, a second dose of the composition can be administered about 7 days, about 14 days or about 28 days following administration of a first dose.

The compositions and methods of employing the compositions of the invention can further include a carrier protein. The carrier protein can be at least one member selected from the group consisting of a tetanus toxoid, a Vibrio cholerae toxoid, a diphtheria toxoid, a cross-reactive mutant of diphtheria toxoid, a E. coli B subunit of a heat labile enterotoxin, a tobacco mosaic virus coat protein, a rabies virus envelope protein, a rabies virus envelope glycoprotein, a thyroglobulin, a heat shock protein 60, a keyhole limpet hemocyanin and an early secreted antigen tuberculosis-6.

“Carrier,” as used herein, refers to a molecule (e.g., protein, peptide) that can enhance stimulation of a protective immune response. Carriers can be physically attached (e.g., linked by recombinant technology, peptide synthesis, chemical conjugation or chemical reaction) to a composition or admixed with the composition.

Carriers for use in the methods and compositions described herein can include, for example, at least one member selected from the group consisting of Tetanus toxoid (TT), Vibrio cholerae toxoid, Diphtheria toxoid (DT), a cross-reactive mutant (CRM) of diphtheria toxoid, E. coli enterotoxin, E. coli B subunit of heat labile enterotoxin (LTB), Tobacco mosaic virus (TMV) coat protein, protein Rabies virus (RV) envelope protein (glycoprotein), thyroglobulin (Thy), heat shock protein HSP 60 Kda, Keyhole limpet hemocyamin (KLH), an early secreted antigen tuberculosis-6 (ESAT-6), exotoxin A, choleragenoid, hepatitis B core antigen, and the outer membrane protein complex of N. meningiditis (OMPC) (see, for example, Schneerson, R., et al., Prog Clin Biol Res 47:77-94 (1980); Schneerson, R., et al., J Exp Med 152:361-76 (1980); Chu, C., et al., Infect Immun 40: 245-56 (1983); Anderson, P., Infect Immun 39:233-238 (1983); Anderson, P., et al., J Clin Invest 76:52-59 (1985); Fenwick, B. W., et al., 54:583-586 (1986); Que, J. U., et al. Infect Immun 56:2645-9 (1988); Que, J. U., et al. Infect Immun 56:2645-9 (1988); (Que, J. U., et al. Infect Immun 56:2645-9 (1988); Murray, K., et al., Biol Chem 380:277-283 (1999); Fingerut, E., et al., Vet Immunol Immunopathol 112:253-263 (2006); and Granoff, D. M., et al., Vaccine 11:Suppl 1:S46-51 (1993)).

Exemplary carrier proteins for use in the methods and compositions described herein can include at least one member selected from the group consisting of Cross-reactive mutant (CRM) of diphtheria toxin including CRM197 (SEQ ID NO: 127); Coat protein of Tobacco mosaic virus (TMV) coat protein (SEQ ID. NO: 128); Coat protein of alfalfa mosaic virus (AMV) (SEQ ID NO: 129); Coat protein of Potato virus X (SEQ ID NO: 130); Porins from Neisseria sp, e.g., class I outer membrane protein of Neisseria meningitides (SEQ ID NO: 131); Major fimbrial subunit protein type I (Fimbrillin) (SEQ ID NO: 132); Mycoplasma fermentans macrophage activating lipopeptide (MALP-2) (SEQ ID NO: 133); p19 protein of Mycobacterium tuberculosis (SEQ ID NO: 134).

The compositions of the invention can further include at least one adjuvant. Adjuvants contain agents that can enhance the immune response against substances that are poorly immunogenic on their own (see, for example, Immunology Methods Manual, vol. 2, I. Lefkovits, ed., Academic Press, San Diego, Calif., 1997, ch. 13). Immunology Methods Manual is available as a four volume set, (Product Code Z37, 435-0); on CD-ROM, (Product Code Z37, 436-9); or both, (Product Code Z37, 437-7). Adjuvants can be, for example, mixtures of natural or synthetic compounds that, when administered with compositions of the invention, such as proteins that stimulate a protective immune response made by the methods described herein, further enhance the immune response to the protein. Compositions that further include adjuvants may further increase the protective immune response stimulated by compositions of the invention by, for example, stimulating a cellular and/or a humoral response (i.e., protection from disease versus antibody production). Adjuvants can act by enhancing protein uptake and localization, extend or prolong protein release, macrophage activation, and T and B cell stimulation. Adjuvants for use in the methods and compositions described herein can be mineral salts, oil emulsions, mycobacterial products, saponins, synthetic products and cytokines. Adjuvants can be physically attached (e.g., linked by recombinant technology, by peptide synthesis or chemical reaction) to a composition described herein or admixed with the compositions described herein.

Therapeutic vaccines designed to treat pre-existing HPV infections are not available. The compositions described herein may have several advantages, such as, reducing the transforming activity of either E6 and E7; E6E7 allows for a greater number of epitopes to be delivered in the vaccine, compared to E6 or E7 alone or peptides derived from these proteins; and production and formulation of a single protein in the vaccine preparation or a blend of E6 and E7 products as multivalent vaccines.

The teachings of all patents, published applications and references cited herein are incorporated by reference in their entirety.

A description of example embodiments of the invention follows.

### EXEMPLIFICATION

### Cloning, Production and Immunogenicity Testing of Recombinant Flagellin-HPV Antigen Fusion Proteins in E. coli

### Cloning and Protein Expression

### Methods:

### DNA Cloning:

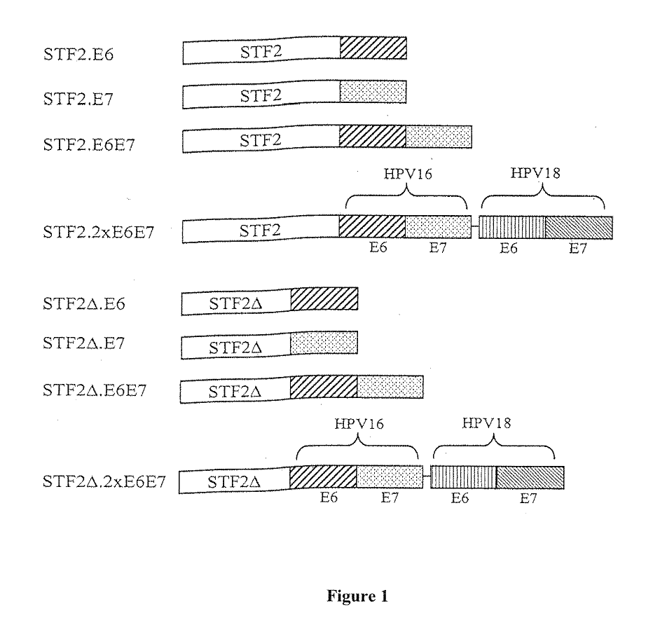

Synthetic genes encoding the E6, E7 and L2 proteins of Human Papilloma Virus strain 16 (HPV 16) were codon optimized for expression in E. coli and synthesized by a commercial vendor (DNA 2.0; Menlo Park, Calif.). The genes were designed to incorporate flanking BlpI sites on both the 5′ and 3′ ends. The gene fragments were excised from the respective plasmids with BlpI and cloned by compatible ends into either the STF2.blp or STF2Δ.blp vector cassette. The fusion constructs incorporating the full-length flagellin gene were designated STF2.E6 (SEQ ID NO: 165, which encodes SEQ ID NO: 209), STF2.E7 (SEQ ID NO: 234) and STF2.L2 (SEQ ID NO: 246) (FIG. 1). The analogous constructs for the flagellin gene lacking a hinge region (also referred to herein as “truncated flagellin”) were designated as STF2Δ.E6 (SEQ ID NO: 191, which encodes SEQ ID NO: 213), STF2 Δ.E7 (SEQ ID NO: 235) and STF2Δ.L2 (SEQ ID NO: 247). In addition, a synthetic gene combining E6 and E7 was fused with STF2 and STF2 Δ to create STF2.E6E7 (SEQ ID NO: 242) and STF2Δ.E6E7 (SEQ ID NO: 243), respectively.

In each case, the constructed plasmids were used to transform competent E. coli TOP10 cells and putative recombinants were identified by PCR screening and restriction mapping analysis. The integrity of the constructs was verified by DNA sequencing, constructs were used to transform the expression host, BLR(DE3) (Novagen, San Diego, Calif.; Cat #69053). Transformants were selected on plates containing kanamycin (50 μg/mL), tetracycline (5 μg/mL) and glucose (0.5%). Colonies were picked and inoculated into 2 mL of LB medium supplemented with 25 μg/mL kanamycin, 12.5 μg/mL tetracycline and 0.5% glucose and grown overnight. Aliquots of these cultures were used to innoculate fresh cultures in the same medium formulation, which were cultured until an optical density (OD_{600 nm})=0.6 was reached, at which time protein expression was induced by the addition of 1 mM IPTG and cultured for 3 hours at 37° C. The cells were then harvested and analyzed for protein expression.

SDS-PAGE and Western Blot: Protein expression and identity were determined by gel electrophoresis and immunoblot analysis. Cells were harvested by centrifugation and lysed in Laemmli buffer. An aliquot of 10 μl of each lysate was diluted in SDS-PAGE sample buffer with or without 100 mM dithiothreitol (DTT) as a reductant. The samples were boiled for 5 minutes and loaded onto a 10% SDS polyacrylamide gel and electrophoresed by SDS-PAGE. The gel was stained with Coomassie R-250 (Bio-Rad; Hercules, Calif.) to visualize protein bands. For Western Blot, 0.5 ml/lane of cell lysate was electrophoresed and electrotransfered onto a PVDF membrane and blocked with 5% (w/v) dry milk.

The membrane was then probed with anti-flagellin antibody (Inotek; Beverly, Mass.). After probing with alkaline phosphatase-conjugated secondary antibody (Pierce; Rockland, Ill.), protein bands were visualized with an alkaline phosphatase chromogenic substrate (Promega, Madison, Wis.). Bacterial clones which yielded protein bands of the correct molecular weight and reactive with the appropriate antibodies were selected for production of protein for use in biological assays and animal immunogenicity experiments.

DNA constructs linking flagellin with HPV 16 antigens are listed in Table 4.

TABLE 4
Flagellin - HPV 16 antigen DNA constructs
for expression in E. coli

| | | Predicted |
|---|---|---|
| | | molecular weight |
| SEQ ID NO: | Construct | (Da) |
| 165 | STF2.E6 | 71,489 |
| 234 | STF2.E7 | 63,323 |
| 242 | STF2.E6E7 | 82,362 |
| 191 | STF2Δ.E6 | 48,495 |
| 235 | STF2Δ.E7 | 40,330 |
| 243 | STF2Δ.E6E7 | 59,369 |
| 246 | STF2.L2 | 103,009 |
| 247 | STF2Δ.L2 | 80,016 |
| 230 | STF2.E6. CTLHis₆ | 55,402 |
| 232 | STF2.4xE6CTLHis₆ | 61,394 |
| 238 | STF2.E7.CTLHis₆ | 55,600 |
| 240 | STF2.4xE7CTLHis₆ | 62,187 |
| 228 | HPV16E6His₆ | 20,010 |
| 237 | HPV16E7His₆ | 11,845 |
| 245 | HPV16E6E7His₆ | 30,833 |
| 249 | HPV16L2His₆ | 51,531 |

### Results:

As assayed by Coomassie blue staining of the SDS-PAGE gel, all the clones displayed a band that migrated at the expected molecular weight. The absence of this band in the control culture (without IPTG) indicates that it is specifically induced by IPTG. Western blotting with antibodies specific for flagellin confirmed that this induced species is the flagellin-HPV antigen fusion protein and suggested that both parts of the fusion protein were expressed intact.

### Purification of STF2.HPV16 E6 (SEQ ID NO: 209)

### Methods

Bacterial growth and cell lysis: STF2.HPV16E6 (SEQ ID NO: 209) was expressed in the E. coli host strain BLR (DE3). E. coli cells were cultured and harvested as described above. The individual strain was retrieved from a glycerol stock and grown in shake flasks to a final volume of 12 liters. Cells were grown in LB medium containing 50 μg/mL kanamycin/12.5 μg/mL tetracycline/0.5% dextrose to OD₆₀₀=0.6 and induced by the addition of 1 mM IPTG for 3 hours at 37° C. The cells were harvested by centrifugation (7000 rpm×7 minutes in a Sorvall RC5C centrifuge) and resuspended in 1×PBS, 1% glycerol, 1 μg/mL DNAse I, 1 mM PMSF, protease inhibitor cocktail and 1 mg/mL lysozyme. The cells were then lysed by two passes through a microfluidizer at 15,000 psi. The lysate was then centrifuged at 45,000×g for one hour to separate soluble and insoluble fractions.

Purification of STF2.HPV16E6 (SEQ ID NO: 209) from E. coli: Following cell lysis and centrifugation (see above) the supernatant (soluble) fraction was collected and supplemented with 50 mM Tris, pH 8. The solution was then applied to a Source Q anion exchange column (GE Healthcare: Piscataway, N.J.) equilibrated in Buffer A (50 mM Tris, pH 8.0+5 mM EDTA). Flow-through and eluate fractions were assayed by SDS-PAGE followed by Coomassie blue staining and Western blotting.

The flagellin-E6 fusion protein did not bind to the column and was found in the flow-through fraction. The flow-through fraction was dialyzed overnight to Buffer B (20 mM citric acid, pH 3.5/8M urea/5 mM EDTA/1 mM beta-mercaptoethanol) and applied to a Source S cation exchange column equilibrated in Buffer B. After eluting with a 5 column-volume linear gradient of 0-1M NaCl in Buffer B, eluate fractions were assayed by SDS-PAGE with Coomassie blue staining and Western blotting. The flagellin-E6 protein did not bind to this column and was again recovered in the flow-through fraction. The flow-through fraction was dialyzed overnight to Buffer C (50 mM Tris, pH 8.0/5 mM EDTA/8M urea) and applied to a Source Q anion exchange column (GE Healthcare; Piscataway, N.J.) equilibrated in Buffer C. After eluting with a 5 column linear gradient of 0-1M NaCl in Buffer C, flow-thru and eluate fractions were assayed by SDS-PAGE followed by Coomassie blue staining and Western blotting. The flagellin-E6 protein did not bind to this column and was again found in the flow-through fraction. The flagellin-E6 protein was refolded by ten-fold dilution into Buffer D (20 mM Tris, pH 8.0/0.15M NaCl/2 mM EGTA/2 μM ZnCl₂) and applied to a Superdex 200 size-exclusion column (GE Healthcare; Piscataway, N.J.) equilibrated in Buffer D. Eluate fractions were assayed by SDS-PAGE followed by Coomassie blue staining and Western blotting. Peak fractions were pooled, sterile filtered and stored at −80° C.

SDS-PAGE and Western Blot analysis: Protein identity and purity of STF2.HPV16 E6 (SEQ ID NO: 209) was determined by SDS-PAGE. An aliquot of 5 of each sample was diluted in SDS-PAGE sample buffer with or without 100 mM DTT as a reductant. The samples were boiled for 5 minutes and loaded onto a 10% polyacrylamide gel (LifeGels; French's Forest, New South Wales, AUS) and electrophoresed. The gel was stained with Coomassie R250 (Bio-Rad; Hercules, Calif.) to visualize protein bands. For western blot, 0.5 μg/lane total protein was electrophoresed as described above and the gels were then electro-transferred to a PVDF membrane and blocked with 5% (w/v) non-fat dry milk before probing with anti-flagellin antibody (Inotek; Beverly, Mass.). After probing with alkaline phosphatase-conjugated secondary antibodies (Pierce; Rockland, Ill.), protein bands were visualized with an alkaline phosphatase chromogenic substrate (Promega; Madison, Wis.).

Protein assay: Total protein concentration for all proteins was determined using the Micro BCA (bicinchonic acid) Assay (Pierce; Rockland, Ill.) in the microplate format, using bovine serum albumin as a standard, according to the manufacturer's instructions.

Endotoxin assay: Endotoxin levels for all proteins were determined using the QCL-1000 Quantitative Chromogenic LAL test kit (Cambrex; E. Rutherford, N.J.), following the manufacturer's instructions for the microplate method.

TLR bioactivity assay: HEK293 cells constitutively express TLR5, and secrete several soluble factors, including IL-8, in response to TLR5 signaling. Cells were seeded in 96-well microplates (50,000 cells/well), and STF2.HPV16 E6 (SEQ ID NO: 209) was added and incubated overnight. The next day, the conditioned medium was harvested, transferred to a clean 96-well microplate, and frozen at −20° C. After thawing, the conditioned medium was assayed for the presence of IL-8 in a sandwich ELISA using an anti-human IL-8 matched antibody pair (Pierce; Rockland, Ill.) #M801E and M802B) following the manufacturer's instructions. Optical density was measured using a microplate spectrophotometer (FARCyte, GE Healthcare; Piscataway, N.J.).

Animal studies: Female C57B/6 mice (Jackson Laboratory, Bar Harbor, Me.) were used at the age of 6-8 weeks. Mice were divided into groups of 6 and received inguinal subcutaneous (s.c.) immunizations on days 0 and 14 as follows:
1. PBS (Phosphate-buffered saline)
2. 3 μg of STF2.HPV16 E6 (SEQ ID NO: 209) in PBS
3. 30 μg STF2.HPV16E6 (SEQ ID NO: 209) in PBS
4. 3 μg of STF2.HPV16E6 (SEQ ID NO: 209) formulated in TITERMAX® Gold adjuvant (CytRx; Norcross, Ga.), according to the manufacturer's instructions.
5. 30 μg of STF2.HPV16 E6 (SEQ ID NO: 209) formulated in TITERMAX® Gold adjuvant.

Seven (7) days following the primary immunization, 2 animals from each group were sacrificed, spleens removed and splenocytes used in ELISPOT assays to analyze antigen-specific immune responses.

Antigen-specific ELISPOT assays: Spleen cells (10⁶ cells/well) from animals 7 days following the primary immunization with of STF2.HPV16 E6 (SEQ ID NO: 209) were added to 96-well Multiscreen-IP plates (Millipore; Billerica, Mass.) coated with anti-IFNγ or IL-5 capture antibody (eBioscience; San Diego, Calif.) diluted in PBS according to the manufacturer's instructions. T cells were then stimulated overnight with naïve antigen-presenting cells (APCs) (10⁶ cells/well) in the absence or presence of a HPV E6—specific antigenic peptide, NH₃-EVYDFAFRDL-COOH (AnaSpec; San Jose, Calif.) (SEQ ID NO: 250). Anti-CD3 (BD Pharmingen; San Jose, Calif.) was used as a positive control at a final concentration of 0.25 μg/ml. Plates were incubated overnight at 37° C./5% CO₂, then washed and incubated with biotinylated detection antibody diluted in PBS/10% fetal bovine serum (FBS) according to the manufacturer's instructions. Plates were developed using the ELISPOT Blue Color Development Module according to the manufacturer's protocol (R&D Systems; Minneapolis, Minn.). Antigen-specific responses were assayed in duplicate from individual animals and quantified using an automated ELISPOT reader (Cellular Technology Ltd.; Cleveland, Ohio). Data is represented as the number of antigen-specific responses/10⁶ APC.

### RESULTS AND DISCUSSION

Protein yield and purity: STF2.HPV16 E6 (SEQ ID NO: 209) was produced in high yield from E. coli cell culture. After purification the yield was about 5.7 mg total protein, the purity was estimated to be greater than about 85% by SDS-PAGE, with an endotoxin level of about 0.97 EU/μg. However, the final size exclusion chromatography (SEC) step in the purification showed the STF2.HPV16 E6 (SEQ ID NO: 209) protein eluting in the void volume (data not shown). This result suggested that the protein is not monomeric and is likely to be aggregated. The STF2.HPV16 E6 (SEQ ID NO: 209) fusion protein showed positive in vitro TLR5 bioactivity, albeit lower than that usually seen for monomeric flagellin-antigen fusion proteins.

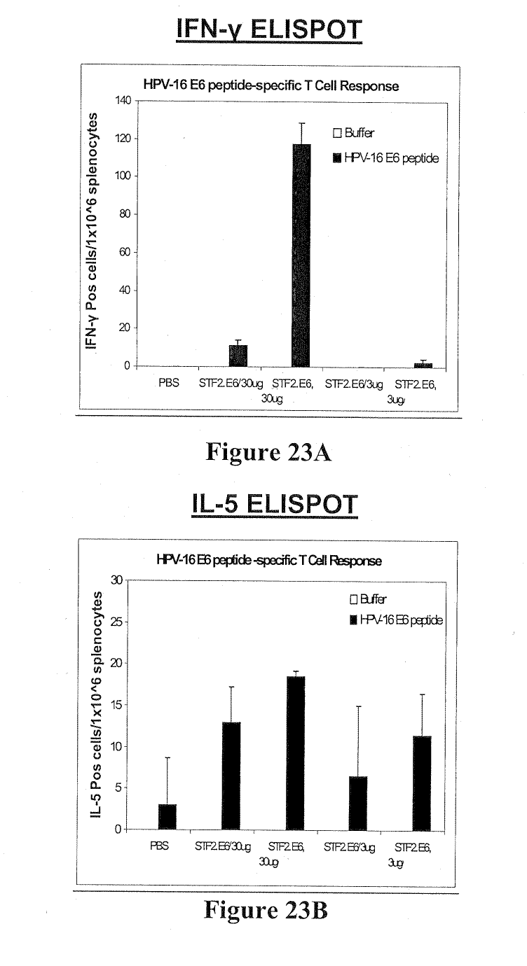

Immunogenicity of STF2.HPV16 E6 (SEQ ID NO: 209): The results of the ELISPOT assay indicate that mice developed antigen-specific T cell responses following a single immunization with STF2.HPV E6 (SEQ ID NO: 209) and that mock-immunized mice did not. Upon stimulation with antigen presenting cells (APCs) primed with the HPV16 E6 peptide (SEQ ID NO: 259), approximately 10 cells/10⁶ splenocytes secreted IFN-γ, vs. none in the mock immunized group. A similar number of cells from animals immunized with STF2.HPV16 E6 (SEQ ID NO: 209) secreted IL-5, vs. approximately 3 in the mock group. Naïve APCs mock-primed with buffer did not stimulate production of IFN-γ or IL-5 in any of the groups. As a positive control, half of the mouse groups were immunized with STF2.HPV16 E6 (SEQ ID NO: 209) protein formulated in TiterMax Gold adjuvant (CytRx; Norcross, Ga.). Not surprisingly, this powerful adjuvant (which is not acceptable for human use) significantly boosted the number of IFN-γ and IL-5 secreting cells (FIGS. 23A and 23B).

One factor which may influence the immune response elicited by STF2.HPV16 E6 (SEQ ID NO: 209) in this experiment is the aggregated state of the STF2.HPV16 E6 (SEQ ID NO: 209) fusion protein, which may hinder the flagellin domain from signaling properly through TLR5. Monomeric STF2.HPV16 E6 (SEQ ID NO: 209) fusion protein may elicit stronger antigen-specific T-cell responses. Furthermore, one or more boosts with the fusion protein may improve the antigen-specific response elicited by STF2.HPV16 E6 (SEQ ID NO: 209) administered without adjuvant. Alternatively, mutation of one or more cysteine residues in the E6 protein to another amino acid may result in an E6 flagellin fusion protein that may have increased immunogenicity due to decreased disulfide bonding in the E6 proteins and, thus, increased exposure of E6 epitopes.

### EQUIVALENTS

While this invention has been particularly shown and described with references to example embodiments thereof, it will be understood by those skilled in the art that various changes in form and details may be made therein without departing from the scope of the invention encompassed by the appended claims.

What is claimed is:

1. A composition that includes at least one protein that includes at least a portion of at least one Toll-like Receptor agonist and at least a portion of at least one papillomavirus tumor suppressor binding protein, wherein the protein activates a Toll-like Receptor.

2. The composition of claim 1, wherein the Toll-like Receptor agonist and the papillomavirus suppressor binding protein are components of a fusion protein.

3. The composition of claim 1, wherein the Toll-like Receptor agonist is a Toll-like Receptor 5 agonist.

4. The composition of claim 3, wherein the Toll-like Receptor 5 agonist includes at least a portion of at least one flagellin.

5. The composition of claim 4, wherein the flagellin lacks at least a portion of a hinge region.

6. The composition of claim 1, wherein the papillomavirus tumor suppressor binding protein includes at least one member selected from the group consisting of at least a portion of an E6 protein and at least a portion of an E7 protein.

7. The composition of claim 1, further including at least a portion of at least one member selected from the group consisting of a papillomavirus transforming protein and a papillomavirus capsid protein.

8. The composition of claim 7, wherein the papillomavirus transforming protein binds at least one member selected from the group consisting of a pRB protein and a p107 protein.

9. The composition of claim 7, wherein the papillomavirus capsid protein includes at least a portion of a major capsid protein.

10. The composition of claim 9, wherein the major capsid protein includes an L1 protein.

11. The composition of claim 7, wherein the papillomavirus capsid protein includes at least a portion of a minor capsid protein.

12. The composition of claim 11, wherein the minor capsid protein includes an L2 protein.

13. A composition that includes at least one protein that includes at least a portion of at least one Toll-like Receptor agonist and at least a portion of at least one papillomavirus transforming protein, wherein the protein activates a Toll-like Receptor agonist.

14. The composition of claim 13, wherein the Toll-like Receptor agonist and the papillomavirus transforming protein are components of a fusion protein.

15. The composition of claim 13, wherein the papillomavirus transforming protein includes at least one member selected from the group consisting of at least a portion of an E6 protein and at least a portion of an E7 protein.

16. A composition that includes at least one protein that includes at least a portion of at least one Toll-like Receptor agonist and at least a portion of at least one papillomavirus capsid protein, wherein the protein activates a Toll-like Receptor.

17. The composition of claim 16, wherein the Toll-like Receptor agonist and the papillomavirus capsid protein are components of a fusion protein.

18. The composition of claim 16, wherein the papillomavirus capsid protein includes at least a portion of at least one papillomavirus major capsid protein.

19. The composition of claim 16, wherein the papillomavirus capsid protein includes at least a portion of at least one papillomavirus minor capsid protein.

20. The composition of claim 19, wherein the papillomavirus minor capsid protein includes at least a portion of an L2 protein.

21. A composition that includes at least a portion of at least one Toll-like Receptor 5 agonist and at least a portion of at least one papillomavirus protein, wherein the papillomavirus protein includes at least one member selected from the group consisting of a papillomavirus E6 protein, a papillomavirus E7 protein and a papillomavirus L2 protein.

22. The composition of claim 21, wherein the Toll-like Receptor 5 agonist and the papillomavirus protein are components of a fusion protein.

23. The composition of claim 21, wherein the Toll-like Receptor 5 agonist includes at least a portion of at least one flagellin.

24. A method of stimulating an immune response in a subject, comprising the step of administering to the subject a composition that includes at least one protein that includes at least a portion of at least one Toll-like Receptor 5 agonist and at least a portion of at least one papillomavirus tumor suppressor binding protein, wherein the protein activates a Toll-like Receptor 5.

25. A method of stimulating an immune response in a subject, comprising the step of administering to the subject a composition that includes at least a portion of at least one Toll-like Receptor 5 agonist and at least a portion of at least one papillomavirus transforming protein, wherein the protein activates a Toll-like Receptor agonist 5.

26. A method of stimulating an immune response in a subject, comprising the step of administering to the subject a composition that includes at least a portion of at least one Toll-like Receptor 5 agonist and at least a portion of at least one papillomavirus capsid protein, wherein the protein activates a Toll-like Receptor 5.

27. A method of stimulating an immune response in a subject, comprising the step of administering to the subject a composition that includes a papillomavirus protein, wherein the papillomavirus protein includes at least a portion of at least a portion of a Toll-like Receptor 5 agonist and at least a portion of at least one member selected from the group consisting of a papillomavirus E6 protein, a papillomavirus E7 protein and a papillomavirus L2 protein.
